(19) United States
(12) Patent Application Publication
(10) Pub. No.: US 2023/0348888 A1
(43) Pub. Date: Nov. 2, 2023

(54) MOLECULAR TRANSPORT FOR VIRAL AGENTS

(71) Applicant: McMaster University, Hamilton (CA)

(72) Inventors: David Bulir, Hamilton (CA); Sylvia Chong, Hamilton (CA)

(21) Appl. No.: 17/923,118

(22) PCT Filed: May 3, 2021
(86) PCT No.: PCT/CA2021/050618

§ 371 Date: Nov. 3, 2022

Related U.S. Application Data
(60) Provisional application No. 63/019,690, filed on May 4, 2020.
(60) Provisional application No. 63/120,142, filed on Dec. 1, 2020.

(51) Int. Cl. C12N 15/10; C12Q 1/70; C12Q 1/6806

(52) U.S. Cl. CPC C12N 15/1003; C12Q 1/70; C12Q 1/6806

(57) ABSTRACT

This application relates to an aqueous mixture composition to collect, transport and store samples suspected of containing a pathogen, the composition comprising a chaotropic agent, a chelator, a detergent, a buffer, and optionally an alcohol. The composition inactivates pathogens and stabilizes nucleic acids and proteins contained within a sample and allows downstream extraction and detection assays to proceed without preprocessing. The composition further allows for sample pooling to facilitate high-throughput detection assays.

### CROSS-REFERENCE TO RELATED APPLICATIONS

This application claims the benefit of priority to U.S. Provisional Application Nos. 63/019,690, filed May 4, 2020, and 63/120,142, filed Dec. 1, 2020, the contents of which are incorporated herein by reference in their entirety.

### FIELD

The present application relates to tools, compositions and methods for collection, transport, and storage of a sample suspected of containing a pathogen and a population of nucleic acids and proteins.

### BACKGROUND

Traditional transport media methods for viral agents such as universal transport media (UTM) or viral transport media (VTM) do not inactivate pathogens, posing added complexity and risk when processing viral samples. In addition to this, these methods are not adaptable to high-throughput screening (HTS) of pooled samples due to inhibitors present in the medium. Newer collection media have been described which kill pathogens, inactivate nucleases, and release polynucleotides from other cellular components within the sample.

### SUMMARY

The present invention describes an aqueous mixture composition for use as a molecular transport medium for the collection, transport, and storage of a sample suspected of containing a pathogen. Pathogens present in samples collected in this medium are inactivated while proteins thereof are denatured and stabilized and nucleic acids thereof are stabilized across a broader range of conditions when compared to universal transport media. The present inventors have shown that the compositions do not require the presence of a reducing agent, and do not require the presence of a mucolytic agent. The absence of a reducing agent is expected to allow for increased shelf life of the compositions relative to compositions comprising a reducing agent such as beta-mercaptoethanol (BME), dithiothreitol (DTT) or Tris (2-carboxyethyl) phosphine (TCEP). In addition, the molecular transport medium is amenable to high-throughput screening (HTS) of pooled samples due to the lack of inhibitors present within the medium. Pooling of samples to increase the testing throughput improves the number of samples a clinical laboratory can process in one day and reduces the amount of extraction reagents used. The low foaming and inactivation of pathogen samples allows for safer use directly from sample transport tube with no pre-processing steps, and provides comparable or improved stability compared to other transport media commonly used.

Accordingly, provided herein is an aqueous composition with a pH between about 6 and about 7 at about 20° C. comprising a chaotropic agent, a chelator, a detergent, and a buffer, wherein the composition does not comprise a reducing agent, wherein the composition does not comprise a mucolytic agent, and wherein the composition inactivates pathogens and stabilizes nucleic acids and/or proteins thereof contained within a sample suspected of containing a pathogen or nucleic acid sequence of interest. In an embodiment, the composition further comprises an alcohol.

In an embodiment, the chaotropic agent is selected from guanidine hydrochloride, guanidine thiocyanate, urea or a combination thereof. In an embodiment, the guanidine thiocyanate is present at a concentration of about 2-6M, about 3-5M, or about 4-6M, optionally about 2M, about 3M, about 4M, or about 6M. In an embodiment, the guanidine hydrochloride is present at a concentration of about 2-8M, about 3-6M, about 4-6M, or optionally about 4M, and/or the urea is present at a concentration of 6-9M, about 7-9M, or optionally about 8M.

In an embodiment, the chelator is selected from EDTA, EGTA, sodium citrate or a combination thereof. In an embodiment, the EDTA is present at a concentration of about 0.01-0.1M, about 0.0.02-0.05M, about 0.02-0.04M, or optionally about 0.02M or about 0.04M. In an embodiment, the chelator is sodium citrate and is present at a concentration of about 0.010-0.1M, about 0.02-0.05, or optionally about 0.025M.

In an embodiment, the alcohol is isopropanol. In an embodiment, the isopropanol is present at a concentration of about 5-40%, about 5-15%, about 10-30%, about 15-25%, or optionally about 10% or about 20%.

In an embodiment, the detergent is Triton X-100. In an embodiment, the Triton X-100 is present at a concentration of about 0.5%-3%, about 1-3%, about 2%-2.5%, or optionally about 1%, about 2.0%, or about 2.5%.

In an embodiment, the buffer is sodium citrate or Tris-HCl and the pH of the buffer is about 7.0. In an embodiment, the Tris-HCl is present at a concentration of about 0.02-0.1M, about 0.025-0.05M or optionally about 0.025M or about 0.05M. In an embodiment, the buffer is sodium citrate, optionally present at a concentration of about 0.02-0.03M or optionally about 0.025M, and/or the pH of the composition is about 6.4.

In an embodiment, the composition comprises 4M guanidine hydrochloride, 8M urea, 2.5% Triton X-100, 0.04M EDTA, and 0.025M sodium citrate and has a pH of ˜6.4 at 20 C.

In an embodiment, the composition comprises 6M guanidine thiocyanate, 2.0% Triton X-100, 0.04M EDTA, and 0.05M Tris-HCl, and has a pH of ˜7.0 at 20 C.

In an embodiment, the composition comprises about 4M guanidine thiocyanate, about 2.0% Triton X-100, about 0.04M EDTA, about 0.05M Tris-HCl, and about 20% isopropanol, and has a pH of about 7.0 at 20 C.

In an embodiment, the composition comprises about 3M guanidine thiocyanate, about 2.0% Triton X-100, about 0.04M EDTA, about 0.05M Tris-HCl, and about 20% isopropanol, and has a pH of about 7.0 at 20 C.

In an embodiment, the composition comprises about 2M guanidine thiocyanate, about 2.0% Triton X-100, about 0.04M EDTA, about 0.05M Tris-HCl, and about 20% isopropanol, and has a pH of about 7.0 at 20 C.

In an embodiment, the composition comprises about 2M guanidine thiocyanate, about 1.0% Triton X-100, about 0.02M EDTA, about 0.025M Tris-HCl, and about 10% isopropanol, and has a pH of about 7.0 at 20 C.

In an embodiment, the composition further comprises a foam suppressor, optionally Antifoam A, optionally present at a concentration of about 0.00001-0.1%, or about 0.1%.

In some embodiments, samples capable of being used in the transport medium include, but are not limited to: nasal swabs, mid-turbinate swabs, nasopharyngeal swabs, nasal sponges, nasal washes, oral swab, buccal swab, throat swab, oral washes or gargles, oropharyngeal swabs, combined oral and nasal swabs, combined throat and nasal swabs, skin swabs, rectal swabs, stool swabs, skin scrapings, nail clippings, urine, blood, saliva, vaginal swabs, meatal swabs, and urethral swabs.

In some embodiments, the pathogen is a bacterium, mycobacterium, fungus, or virus. In some embodiments, the pathogen is a virus. In some embodiments, the pathogen is a respiratory virus. In some embodiments, the pathogen is a coronavirus. In a particular embodiment, the coronavirus is SARS-CoV-2.

In some embodiments, the nucleic acids are stable at about 4° C. for up to 14 days, up to 30 days, up to 60 days, up to 90 days, up to 120 days, up to 150 days, up to 180 days, up to 210 days, or up to 270 days. In some embodiments, the nucleic acids are stable at about room temperature for up to 14 days, up to 30 days, up to 60 days, up to 90 days, up to 120 days, or up to 180 days. In some embodiments, the nucleic acids are stable at about 37° C. for up to 14 days.

In some embodiments, the proteins are stable at about 4° C. for up to 14 days, up to 30 days, or up to 60 days. In some embodiments, the proteins are stable at about room temperature for up to 14 days, up to 30 days, or up to 60 days. In some embodiments, the proteins are stable at about 37° C. for up to 14 days.

In some embodiments, the composition is used to collect, transport and store samples suspected of containing a pathogen. In some embodiments, the composition is used as a molecular transport medium. In some embodiments, the molecular transport medium is used as part of a kit.

Another aspect of the disclosure includes a method of inactivating pathogens and stabilizing nucleic acids thereof contained within a sample suspected of containing a pathogen, the method comprising: obtaining a sample suspected of containing a pathogen; contacting the sample with a composition described herein; and incubating the sample contacted with the composition under conditions to allow for inactivation of pathogens and stabilization of nucleic acids.

In some embodiments, stabilization of the nucleic acids allows extraction and detection assays without preprocessing. In some embodiments, stabilization of the nucleic acids allows for sample pooling to facilitate high-throughput detection assays. In some embodiments, stabilization of the nucleic acids enhances the performance of detection assays.

In some embodiments, the composition facilitates nucleic acid binding to silica for purification.

Another aspect of the disclosure includes a method of inactivating pathogens and denaturing and stabilizing proteins thereof contained within a sample suspected of containing a pathogen, the method comprising: obtaining a sample suspected of containing a pathogen; contacting the sample with a composition described herein; and incubating the sample contacted with the composition under conditions to allow for inactivation of pathogens and denaturing and stabilization of proteins.

In some embodiments, the stabilization of the proteins allows for the detection of a protein such as a pathogen protein.

A further aspect of the disclosure includes a kit for collection, transport and/or storage of a sample suspected of containing a pathogen, the kit comprising: a composition described herein, and a container.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

The embodiments of the application will now be described in greater detail with reference to the attached drawings in which:

FIG. 1A-B shows images of Vero cells at 5 days post addition of RNA (FIG. 1A) and 14 days post addition of RNA (FIG. 1B). No cytopathic effect was observed.

FIG. 2A-C show nucleic acid stability data over time for samples stored at room temperature (FIG. 2A), 4° C. (FIG. 2B), and 37° C. (FIG. 2C).

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used herein, the term “stable” refers to a composition or component therein that remains relatively unchanged for example does not degrade appreciably or retains a desired activity or characteristic over a period of time. For example, the phrase “shelf-stable” when used to refer to the compositions described herein means that the composition retains the ability to inactivate pathogens and stabilize nucleic acids thereof contained within a sample when stored under ambient conditions (e.g. on a shelf). Similarly, nucleic acids are described as being stable in the compositions described herein when the crossing threshold is within +/−3.0 cycle difference from time zero, when detected by PCR.

The term “crossing threshold” as used herein refers to the cycle number at which the accumulated fluorescence in a polymerase chain reaction (PCR) crosses the threshold above which the fluorescent signal of a PCR product can be detected above the background signal. As used herein, crossing threshold refers to the same value as defined by cycle threshold (Cₜ) or cycle quantitation (C_{q}).

The term “nucleic acid” as used herein refers to a polynucleotide, such as deoxyribonucleic acid (DNA), ribonucleic acid (RNA), modified nucleotides and/or nucleotide derivatives, including synthetic nucleic acids and DNA/RNA hybrids.

The term “protein” as used herein refers to a polypeptide comprising a sequence of amino acids and includes unmodified proteins as well as proteins modified for example by phosphorylation, glycosylation, acetylation, methylation and/or lipidation. The protein may comprise one or more antibody binding sites and may be detected through antigen testing, or other means such as mass spectrometry.

The term “sample” as used herein refers to any material in which the presence or amount of one or more components therein is unknown and can be determined in an assay. The sample may be for example a human or animal sample, including clinical samples and swabs, or an environmental sample including for example a surface swab or waste water sample. The sample may comprise cellular and non-cellular material, including, but not limited to, tissue samples, saliva, sputum, urine, blood, serum, other bodily fluids and/or secretions. The sample may be collected in any suitable manner and/or by any suitable person, including self-collected or collected by a health care professional. Any type of suitable sample may be used with the compositions and methods described herein, including but not limited to, a nasal swab, mid-turbinate swab, nasopharyngeal swab, nasal sponge, nasal wash, oral swab, oral wash or gargle, buccal swab, throat swab, oropharyngeal swab, combined oral and nasal swab, combined throat and nasal swab, skin swab, rectal swab, stool swab, skin scraping, nail clipping, urine sample, blood sample, saliva sample, vaginal swab, meatal swab, or urethral swab. In an embodiment, the sample is a nasal swab, mid-turbinate swab, nasopharyngeal swab, nasal sponge, nasal wash, throat swab, oral wash or gargle, combined oral and nasal swab, or combined throat and nasal swab.

As used herein, the term “pathogen” means an organism of clinical significance, for example a disease-causing microorganism. Pathogens include, but are not limited to bacteria, mycobacteria, fungi, and viruses, such as Influenza A, Influenza A—H1 subtype, Influenza A—H3 subtype, Influenza A 2009 H1N1 subtype, Influenza A—H5, Influenza B, Respiratory Syncytial Virus A, Respiratory Syncytial Virus B, Parainfluenza 1, Parainfluenza 2, Parainfluenza 3, Parainfluenza 4, Human Bocavirus, Human Metapneumovirus, Rhinovirus/Enterovirus, Adenovirus, Coronavirus HKU1, Coronavirus NL63, Coronavirus OC43, Coronavirus 229E, Chlamydophila pneumoniae, Legionella pneumophila, Mycoplasma pneumoniae, Listeria monocytogenes, Strep pneumo, Group A Streptococcus (GAS), Astrovirus, SARS-CoV-1, SARS-CoV-2, MERS, HSV1/2, M. tuberculosis, M. avium, Trichophyton spp., Microsporidium spp., Chlamydophila trachomatis, Neisseria spp. In an embodiment, the pathogen is a virus such as a coronavirus, optionally SARS-CoV-2.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

### II. Compositions and Uses

The compositions described herein comprise a chaotropic agent, a chelator, a detergent, and a buffer, but lack a reducing agent and lack a mucolytic agent. The compositions described herein may further comprise an alcohol. The inventors have found that the compositions described herein are useful for inactivating pathogens and stabilizing nucleic acids and/or proteins thereof contained within a sample suspected of containing a pathogen. The compositions described herein are therefore useful as a molecular transport medium i.e. a medium for the collection and transport of a sample comprising a nucleic acid. The compositions described herein are also useful for the collection, transport, and/or storage of a sample suspected of containing a pathogen.

Each component provides a specific function.

Chaotropic agents help denature proteins to inactivate a wide range of pathogens, including viral, bacterial, fungal, and mycobacterium. As used herein, “chaotrope” or “chaotropic agent” means a molecule that disrupts the hydrogen bonding structure of water, thereby disrupting the native structure of macromolecules such as proteins and nucleic acids. The chaotropic agents of the compositions disclosed herein do not act as reducing agents or mucolytic agents, or are used at concentrations at which there is no reducing activity or mucolytic activity. Chaotropic agents include, but are not limited to guanidine hydrochloride, guanidine thiocyanate, urea, n-butanol, ethanol, lithium perchlorate, lithium acetate, magnesium chloride, phenol, 2-propanol, sodium dodecyl sulfate, thiourea, potassium iodide, and sodium iodide. In an embodiment, the chaotropic agent is guanidine hydrochloride, guanidine thiocyanate, urea, or a combination thereof. Any suitable concentration of chaotrope may be used and will depend on the chaotrope. In an embodiment, the concentration of guanidine hydrochloride is about 2-8M, about 3-6M, about 4-6M, or optionally about 4M. In an embodiment, the concentration of guanidine thiocyanate is about 2-6M, about 3-5M, or about 4-6M. In an embodiment, the concentration of guanidine thiocyanate is about 2M. In an embodiment, the concentration of guanidine thiocyanate is about 3M. In an embodiment, the concentration of guanidine thiocyanate is about 4M. In an embodiment, the concentration of guanidine thiocyanate is about 6M. In an embodiment, the concentration of urea is about 6-9M, about 7-9M, or optionally about 8M.

Chelators can be used to help sequester divalent cations that are important cofactors for RNAse activity. As used herein, “chelator” means a molecule that binds or sequesters a ligand, such as a cationic divalent metal. The chelators of the compositions disclosed herein do not act as reducing agents or mucolytic agents, or are used at concentrations at which there is no reducing activity or mucolytic activity. Any suitable chelator may be used, including but not limited to, ethylenediaminetetraacetic acid (EDTA), EDTA-OH, ethylene glycol-bis(β-aminoethyl ether)-N,N,N′,N′-tetraacetic acid (EGTA), sodium citrate, nitrilotriacetic acid (NTA), trans-1,2-Diaminocyclohexane-N,N,N′,N′-tetraacetic acid (cyDTA), diethylenetriaminepentaacetic acid (DTPA), O,O′-Bis(2-aminoethyl)ethyleneglycol-N,N,N′,N′-tetraacetic acid (GEDTA), Iminodiacetic acid (IDA), N-(2-Hydroxyethyl)iminodiacetic acid (HIDA). In an embodiment, the chelator is EDTA, EGTA, sodium citrate, or a combination thereof. Any suitable concentration of chelator may be used and will depend on the chelator. In an embodiment, the chelator is EDTA and is present at a concentration of about 0.01-0.1M, about 0.02-0.05M, about 0.02-0.04M, or optionally about 0.02M or about 0.04M. In an embodiment, the chelator is sodium citrate and is present at a concentration of about 0.010-0.1M, about 0.02-0.05, or optionally about 0.025M.

Alcohols help stabilize nucleic acids. Alcohols can also help facilitate nucleic acid binding to silica. Furthermore, higher concentration of alcohols reduces the surface tension of the liquids, reducing foaming to make samples more amenable to automated assay platforms. As used here in, “alcohol” means a simple short chain monoalcohol such as methyl, ethyl, n-propyl, isopropyl, and butyl alcohol. In an embodiment, the alcohol is isopropanol. The alcohols of the compositions disclosed herein do not act as reducing agents or mucolytic agents, or are used at concentrations at which there is no reducing activity or mucolytic activity. A combination of two or more alcohols, for example ethanol and isopropanol, may be used. Any suitable concentration of alcohol may be used and will depend on the alcohol. In an embodiment, the alcohol is isopropanol and is present at a concentration of about 5-40%, about 5-15%, about 10-30%, about 15-25%, or optionally about 10% or about 20% (vol/vol).

Detergents are helpful for samples that are difficult to lyse such as oral secretions and nasal secretions. As used herein, “detergent” means an amphiphilic surfactant. The detergents of the compositions disclosed herein do not act as reducing agents or mucolytic agents, or are used at concentrations at which there is no reducing activity or mucolytic activity. Detergents include anionic detergents, e.g. deoxycholic acid, sodium lauroyl sarcosinate (INCI), sodium dodecyl sulfate (SDS), cationic detergents such as cetyltrimethylammonium bromide (CTAB), and non-ionic/zwitterionic detergents such as Triton X-100, tween-20, and 3-cholamidopropyl dimethylammonio 1-propanesulfonate (CHAPS). In an embodiment, the detergent is Triton X-100. Any suitable concentration of detergent may be used and will depend on the detergent. In an embodiment, the detergent is Triton X-100 and is present at a concentration of about 0.5%-3%, about 1-3%, or about 2%-2.5%. In an embodiment the Triton X-100 is present at a concentration of about 1.0%. In an embodiment the Triton X-100 is present at a concentration of about 2.0%. In an embodiment the Triton X-100 is present at a concentration of about 2.5%.

Buffers are helpful to keep the compositions within the desired pH range. As used herein, “buffer” means an aqueous mixture of an acid and its conjugate base that minimize changes in pH caused by the addition of an acid or base when near their pKa. The buffers of the compositions disclosed herein do not act as reducing agents or mucolytic agents, or are used at concentrations at which there is no reducing activity or mucolytic activity. Any suitable buffer may be used. The pH chosen for the buffer is related to its pKa, however, lower pH compositions could help facilitate nucleic acid binding silica for purification applications. Suitable buffers may include, but are not limited to, sodium citrate, 2-ethanesulfonic acid (MES), 3-(N-morpholino)propanesulfonic acid (MOPS), 4-(2-hydroxyethyl)-1-piperazineethanesulfonic acid (HEPES), or any other Good's buffer with an appropriate pKa value such as about between 5-9. In an embodiment, the buffer is sodium citrate or Tris-HCl. The selection of buffer will depend in part on the desired pH of the composition. In an embodiment, the buffer is sodium citrate and the composition has a pH of about 6.4 at 20 C. In an embodiment, the buffer is Tris-HCl and the composition has a pH of about 7.0 at 20 C. Any suitable concentration of buffer may be used, and will depend on the buffer. In an embodiment, the buffer is sodium citrate and is present at a concentration of about 0.02-0.03M or optionally about 0.025M. In an embodiment, the buffer is Tris-HCl and is present at a concentration of about 0.02-0.1M, about 0.025-0.05M or optionally about 0.025M or about 0.05M.

The compositions described herein do not comprise a reducing agent and do not comprise a mucolytic agent. As used herein, “reducing agent” means a compound which acts to reduce another compound in a redox reaction, for example by acting as an electron donor. Common reducing agents include dithiothreitol (DTT), beta-mercaptoethanol (BME), N-acetylcysteine (NAC), and Tris (2-carboxyethyl) phosphine (TCEP). Without wishing to be bound by theory, the lack of a reducing agent in the compositions described herein confers a performance benefit for example with respect to the long-term stability of the composition, as reducing agents such as those listed above are notoriously unstable in solution. Accordingly, the compositions described herein may be shelf-stable for up to one month, two months, three months, four months, or longer. As used herein, “mucolytic agent” means a compound which acts to reduce the viscosity of a biological sample comprising mucous. Common mucolytic agents include reducing agents such as those listed above, as well as ascorbic acid, dithionite, erythiorbate, cysteine, glutathione, dierythritol, a resin-supported thiol, a resin-supported phosphine, vitamin E, and/or trolox, or salts thereof, sodium citrate, potassium citrate, potassium iodide, ammonium chloride, guaiphenesin (or guaifenesin), Tolu balsam, Vasaka, ambroxol, carbocisteine, erdosteine, mecysteine, and domase alfa. The components of the compositions disclosed herein do not act as mucolytic agents or reducing agents, or are used at concentrations at which there is no reducing activity or mucolytic activity.

Foam suppressors such as Antifoam A may also be included for the purpose of reducing foam to make samples more amenable to automated assay platforms. In an embodiment, the composition further comprises Antifoam A, optionally at a concentration of about 0.00001-0.1%, optionally about 0.1%.

In an embodiment described herein as DB #1, the composition comprises 4M guanidine hydrochloride, 8M urea, 2.5% Triton X 100, 0.04M EDTA, and 0.025M sodium citrate and has a pH of ˜6.4 at 20 C.

In an embodiment described herein as DB #2, the composition comprises 4M guanidine hydrochloride, 8M urea, 2.5% Triton X-100, 0.04M EDTA, 0.025M sodium citrate, and 0.10% Antifoam A, and has a pH of ˜6.4 at 20 C.

In an embodiment described herein as DB #3, the composition comprises 6M guanidine thiocyanate, 2.0% Triton X-100, 0.04M EDTA, and 0.05M Tris-HCl, and has a pH of ˜7.0 at 20 C.

In an embodiment described herein as DB #4, the composition comprises 6M guanidine thiocyanate, 2.0% Triton X-100, 0.04M EDTA, 0.05M Tris-HCl, and 0.1% Antifoam A, and has a pH of ˜7.0 at 20 C.

In an embodiment described herein as DB #5, McMaster Molecular Medium, or MMM, the composition comprises 4M guanidine thiocyanate, 2.0% Triton X-100, 0.04M EDTA, 0.05M Tris-HCl, and 20% isopropanol, and has a pH of ˜7.0 at 20 C.

In an embodiment described herein as DB #6, the composition comprises 4M guanidine thiocyanate, 2.0% Triton X-100, 0.04M EDTA, 0.05M Tris-HCl, 20% isopropanol, and 0.1% Antifoam A, and has a pH of ˜7.0 at 20 C.

In an embodiment, the composition comprises about 3M guanidine thiocyanate, about 2.0% Triton X-100, about 0.04M EDTA, about 0.05M Tris-HCl, and about 20% isopropanol, and has a pH of about 7.0 at 20 C.

In an embodiment, the composition comprises about 2M guanidine thiocyanate, about 2.0% Triton X-100, about 0.04M EDTA, about 0.05M Tris-HCl, and about 20% isopropanol, and has a pH of about 7.0 at 20 C.

In an embodiment, the composition comprises about 2M guanidine thiocyanate, about 1.0% Triton X-100, about 0.02M EDTA, about 0.025M Tris-HCl, and about 10% isopropanol, and has a pH of about 7.0 at 20 C.

Other components may also be included, for example a nucleic acid binding material (including, but not limited to, silica, anion exchange resin, or paramagnetic particles, optionally comprising surface modifications such as silanol, epoxide, diol, and carboxyl groups).

The constituents of the composition, as well as the concentration and pH may have an impact on downstream applications. For example, DB #1/DB #2 show similar performance to DB #5 (McMaster Molecular Media) in terms of stability. However, when used in experiments where multiple samples are mixed (‘pooled’) together prior to nucleic acid extraction, DB #5 (McMaster Molecular Media) shows comparable crossing thresholds compared to unpooled samples, whereas DB #1 and DB #2 show a later crossing threshold compared to unpooled samples when used in the same application and with the same samples. Nevertheless, in other applications where lower pH of the buffer could help facilitate for example binding to silica for downstream purification or other applications, DB #1/DB #2 could be particularly suitable.

The compositions described herein are useful for the collection, transport and storage of samples suspected of containing a pathogen. Accordingly, in an embodiment, the compositions described herein are used for the collection, transport, and/or storage of a sample suspected of containing a pathogen.

As demonstrated herein, nucleic acids in samples stored in the compositions described herein are stable over time. For example, nucleic acids remain stable in samples stored at about 4 C for up to about 7 days, up to about 14 days, up to about 30 days, up to about 60 days, up to about 90 days, up to about 120 days, up to about 150 days, up to about 180 days, up to about 210 days, or up to about 270 days. Accordingly, in an embodiment, the composition is suitable for and/or is used for storage of a sample for up to about 14 days, up to about 30 days, up to about 60 days, up to about 90 days, up to about 120 days, up to about 150 days, up to about 180 days, up to about 210 days, or up to about 270 days, or longer, at a temperature of about 4 C prior to extraction. Nucleic acids remain stable in samples stored at about room temperature for up to about 7 days, up to about 14 days, up to about 30 days, up to about 60 days, up to about 90 days, up to about 120 days, up to about 150 days, or up to about 180 days. Accordingly, in an embodiment, the composition is suitable for and/or is used for storage of a sample for up to about 14 days, up to about 30 days, up to about 60 days, up to about 90 days, up to about 120 days, up to about 150 days, or up to about 180 days, or longer, at a temperature of between about 4 C to about 25 C prior to extraction. Nucleic acids remain stable in samples stored at about 37 C for up to about 7 days or up to about 14 days. Accordingly, in an embodiment, the composition is suitable for and/or is used for storage of a sample for up to about 7 days or up to about 14 days at a temperature between about 4 C to about 37 C.

Proteins in samples stored in the compositions described herein are also expected to be denatured and protected from protein degradation (stabilized) over time.

### III. Methods

As demonstrated herein, the compositions described herein can be used in methods of inactivating pathogens and stabilizing nucleic acids and/or proteins thereof contained within a sample suspected of containing a pathogen. Accordingly, in an embodiment, there is provided a method of inactivating pathogens and stabilizing nucleic acids contained within a sample suspected of containing a pathogen, the method comprising: obtaining a sample suspected of containing a pathogen; contacting the sample with a composition disclosed herein; and incubating the sample contacted with the composition under conditions to allow for inactivation of pathogens and stabilization of nucleic acids. The sample may be contacted with the composition using any suitable method, for example by immersing the sample in the solution, and optionally mixing the sample with the composition by swirling, inverting, or vortexing.

The inactivation of pathogens by the compositions described herein may allow for downstream applications such as nucleic acid purification and detection and/or antigen testing to be performed safely without the need for the additional safety precautions that may be required when handling biological or clinical samples, such as aliquoting samples in a biosafety hood. Any suitable incubation time and temperature may be used for inactivating pathogens and stabilizing nucleic acids and/or proteins. As shown herein, pathogens such as SARS-CoV-2 are inactivated after an incubation period of as little as 15 minutes at room temperature. Accordingly, in an embodiment, the sample is incubated with the composition for at least about 10 minutes, at least about 15 minutes, at least about 30 minutes, at least about 45 minutes, or longer. In an embodiment, the sample is incubated with the composition at a temperature between about 4 C and about 100 C, such as about 4 C, about 10 C, about 15 C, about 20 C, about 25 C, about 30 C, about 37 C, about 42 C, about 65 C, about 72 C, about 95 C, about 100 C, or any other temperature in between about 4 C and about 100 C. Optionally, the sample is incubated with the composition at about room temperature, or at about 37 C.

Samples stored in the compositions described herein can be used directly in downstream applications such as nucleic acid extraction and detection and/or antigen testing, without requiring additional steps such as the addition of a lysis buffer and subsequent incubation. As demonstrated herein, the compositions described herein can be used in place of lysis buffer, for example easyMag® (bioMerieux) lysis buffer, for nucleic acid extraction using for example easyMag extraction reagents and protocols. Furthermore, samples lysed in the compositions described herein show improved extraction compared to samples lysed in easyMag lysis buffer when detected by PCR-based methods. Accordingly, in an embodiment, also provided herein is a method of extracting nucleic acids from a sample contained within a sample suspected of containing a pathogen, the method comprising: obtaining a sample suspected of containing a pathogen; contacting the sample with a composition disclosed herein; incubating the sample contacted with the composition under conditions to allow for inactivation of pathogens and stabilization of nucleic acids, and extracting the nucleic acids directly from the sample contacted with a composition described herein. In an embodiment, extracting the nucleic acids does not require any additional lysis reagents. Any suitable extraction method may be used, for example those relying on the presence of a high concentration of ion, chaotrope, or alcohol to facilitate for example binding to the substrate. Common extraction methods include solid phase extraction such as binding to silica (including for example silica/diatomaceous earth, or silica-coated particles), or ion exchange resins, or precipitation based methods such as ethanol precipitation. Commercially available kits and/or reagents for high-throughput/automated nucleic acid extraction include for example easyMag (bioMerieux), HT Viral TNA Kit (Promega), and MagMax (Thermofisher). Silica spin columns, for example Monarch™ Total RNA miniprep (NEB), or anion exchange resins, for example Viral RNA columns (Qiagen) may also be used.

As demonstrated herein, nucleic acids in samples stored in the compositions described herein are stable over time. Accordingly, in an embodiment, the sample is optionally stored in the composition prior to extraction. For example nucleic acids remain stable in samples stored at about 4 C for up to about 7 days, up to about 14 days, up to about 30 days, up to about 60 days up to about 90 days, up to about 120 days, up to about 150 days, up to about 180 days, up to about 210 days, or up to about 270 days. Accordingly, in an embodiment, the sample is stored for up to about 14 days, up to about 30 days, up to about 60 days, up to about 90 days, up to about 120 days, up to about 150 days, up to about 180 days, up to about 210 days, or up to about 270 days, or longer, at a temperature of about 4 C prior to extraction. Nucleic acids remain stable in samples stored at about room temperature for up to about 7 days, up to about 14 days, up to about 30 days, up to about 60 days, up to about 90 days, up to about 120 days, up to about 150 days, or up to about 180 days. Accordingly, in an embodiment, the composition is suitable for and/or is used for storage of a sample for up to about 14 days, up to about 30 days, up to about 60 days, up to about 90 days, up to about 120 days, up to about 150 days, or up to about 180 days, or longer, at a temperature of between about 4 C to about 25 C prior to extraction. Nucleic acids remain stable in samples stored at about 37 C for up to about 7 days or up to about 14 days. Accordingly, in an embodiment, the sample is stored for up to about 7 days or up to about 14 days at a temperature between about 4 C to about 37 C,

Nucleic acids stabilized and extracted from a sample using the methods described herein can be detected by any suitable methods for example PCR-based methods (such as qPCR, digital droplet PCR, digital PCR, RT-qPCR), NextGen sequencing, isothermal amplification methods, or FISH. Accordingly, also provided is a method of detecting the presence of a pathogen in a sample, the method comprising: obtaining a sample suspected of containing a pathogen; contacting the sample with a composition disclosed herein; incubating the sample contacted with the composition under conditions to allow for inactivation of pathogens and stabilization of nucleic acids, extracting the nucleic acids directly from the sample contacted with a composition described herein, and detecting the nucleic acids, optionally by subjecting the sample to PCR.

Samples collected and/or stored in the compositions described herein can be pooled for high-throughput screening applications. As shown herein, the presence of nucleic acids from a single sample can be detected when up to 8 samples are pooled prior to extraction. Accordingly, in an embodiment, at least two, at least four, at least 8, or more than eight samples are pooled prior to extraction.

Proteins in samples stored in the compositions described herein are also expected to be stable over time. Accordingly, the compositions described herein are expected to be useful for antigen testing for example for a protein such as a pathogen protein in a sample. Proteins from a sample stabilized using the compositions and methods described herein can be detected by any suitable antigen testing methods such as for example ELISA, Western blotting, lateral flow antigen testing, etc. Any other suitable antigen testing method may be used. Proteins from a sample stabilized using the compositions and methods described herein may also be identified by other methods including for example mass spectroscopy. Accordingly, also provided is a method of detecting the presence of a pathogen protein in a sample, the method comprising: obtaining a sample suspected of containing a pathogen; contacting the sample with a composition disclosed herein; incubating the sample contacted with the composition under conditions to allow for inactivation of pathogens and stabilization of proteins, and detecting the pathogen protein.

### IV. Kits

The compositions described herein may be provided as a component in a kit, for example a kit for the collection, transport, and/or storage of a sample suspected of containing a pathogen. Any suitable components may be included in the kit, for example a container for housing the composition and/or collecting the sample. Any suitable container may be used, for example a test tube or a vial. Other suitable containers are known in the art and will depend on the type of sample being collected.

The kit may also comprise a collection device for the collection of a sample suspected of containing a pathogen. Suitable collection devices are known in the art and will depend on the type of sample being collected. Suitable collection devices may include for example swabs, curettes, loops, or funnels.

The kit may also comprise a wash solution suitable for obtaining samples such as nasal washes, oral washes or gargles, or similar samples. Suitable wash solutions may include for example a saline solution.

### EXAMPLES

The following non-limiting examples are illustrative of the present application:

### Example 1. Compositions of Molecular Transport Media

The compositions of various molecular transport media (designated DB #1-6) made and demonstrated herein are described below. The temperature of the composition is typically maintained between about 25-30 C to facilitate the dissolution of the components, in particular the chaotrope, and then cooled to about 20 C before adjusting the pH and final volume.

| | Desired Final | |
|---|---|---|
| DB #1 | Concentration (M) | Concentration (%) |
| Guanidine Hydrochloride | 4.00 | |
| Urea | 8.0 | |
| Triton X-100 | | 2.5 |
| EDTA | 0.040 | |
| Sodium Citrate | 0.025 | |

pH = ~6.4 @ 20° C.

| | Desired Final | |
|---|---|---|
| DB #2 | Concentration (M) | Concentration (%) |
| Guanidine Hydrochloride | 4.00 | |
| Urea | 8.0 | |
| Triton X-100 | | 2.5 |
| EDTA | 0.040 | |
| Sodium Citrate | 0.025 | |
| Antifoam A | | 0.1 |

pH = ~6.4 @ 20° C.

| | Desired Final | |
|---|---|---|
| DB #3 | Concentration (M) | Concentration (%) |
| Guanidine Thiocyanate | 6.0 | |
| Triton X-100 | | 2.0 |
| EDTA | 0.040 | |
| TRIS-HCl | 0.050 | |

pH = ~7.0 @ 20° C.

| | Desired Final | |
|---|---|---|
| DB #4 | Concentration (M) | Concentration (%) |
| Guanidine Thiocyanate | 6.0 | |
| Triton X-100 | | 2.0 |
| EDTA | 0.040 | |
| TRIS-HCl | 0.050 | |
| Antifoam A | | 0.1 |

pH = ~7.0 @ 20° C.

| DB #5 | | |
|---|---|---|
| (McMaster Molecular | | |
| Medium, MMM, also | Desired Final | |
| called MMM#4) | Concentration (M) | Concentration (%) |
| Guanidine Thiocyanate | 4.0 | |
| Triton X-100 | | 2.0 |
| EDTA | 0.040 | |
| TRIS-HCl | 0.050 | |
| Isopropanol | | 20 |

pH = ~7.0 @ 20° C.

| | Desired Final | |
|---|---|---|
| DB #6 | Concentration (M) | Concentration (%) |
| Guanidine Thiocyanate | 4.0 | |
| Triton X-100 | | 2.0 |
| EDTA | 0.040 | |
| TRIS-HCl | 0.050 | |
| Isopropanol | | 20 |
| Antifoam A | | 0.1 |

pH = ~7.0 @ 20° C.

### Example 2. Sample Stability in Molecular Transport Media

Methods: A clinical COVID-19-positive nasopharyngeal sample was collected in 1 mL UTM (COPAN) and spun at 13,000 rpm for 5 mins. The cell pellet was washed in phosphate buffered saline (PBS) twice before resuspension in 500 μL TE buffer. Serial dilutions of cell suspension were made in the 6 DB media compositions described in this application (Example 1). The sample was also suspended in bioMerieux Lysis Buffer, eNAT™ (COPAN) and Tris-EDTA (TE) buffer as controls. Samples in the various media were extracted at days 0, 1, 3, 7, and 14 via easyMAG® and tested in a real-time reverse transcriptase polymerase chain reaction (RT-PCR) triplex assay targeting E gene, UTR, and RNAse P (as a sample adequacy/control marker) to detect SARS-CoV-2 RNA.

The sample stability was assessed by comparing crossing threshold (Cₜ) data, described in Tables 2-6, to determine relative performance of the different media.

At day 0 (Table 2) the Cₜ scores are comparable regardless of the medium the samples are stored in.

At day 1, room temperature (Table 3), eNAT™, Lysis Buffer and DB compositions #1-6 have comparable Cₜ scores. However, UTM and TE buffer have later Cₜ thresholds at dilutions of 10⁻³, 10⁻⁴ and 10⁻⁵ in comparison with a smaller difference at 10⁻⁶, which could be due to this Cₜ being close to the limit of detection.

At day 1, 4° C. (Table 3), eNAT™, Lysis Buffer, UTM and DB #1-6 have comparable Cₜ thresholds. Therefore, UTM at 4° C. is more stable than at room temperature. TE buffer yields later crossing thresholds regardless of the storage temperature.

At day 3, room temperature (Table 4), eNAT™, Lysis Buffer and DB #1-6 have comparable Cₜ thresholds, while UTM and TE buffer show later crossing thresholds.

At day 3, 4° C. (table 4), eNAT™, Lysis Buffer, UTM and DB #1-6 have comparable Cₜ thresholds. Therefore, UTM at 4° C. is more stable than at room temperature. TE buffer yields later crossing thresholds regardless of the storage temperature.

After 7 days at room temperature (Table 5), the DB #1-6 have more favorable Cₜ thresholds at higher sample concentrations. DB buffers 1 and 2 are comparable if not marginally better compared to eNAT™. DB buffer 3 is comparable to eNAT™ with E gene and UTR, however, at lower dilution, eNAT™ yielded an earlier Cₜ in one UTR replicate. DB buffers 4-6 are comparable to eNAT™, TE buffer resulted in later Cₜ thresholds. UTM also resulted in later Cₜ thresholds for UTR but identical at 10⁻⁵ and 10⁻⁶ concentrations for E gene. Lysis buffer and eNAT™ had comparable Cₜ thresholds.

At day 7, 4° C. (table 5), DB #1 and 4-6 were identical to eNAT™ for both UTR and E gene. DB #2 was identical to eNAT™ for E gene and mostly identical to UTR but at lower dilutions the Cₜ was earlier with eNAT™. DB #3 is identical to eNAT™ for E gene and mostly identical for UTR except at lower concentrations, Cₜ marginally slower than eNAT™. TE buffer and UTM resulted in a later Cₜ. Lysis buffer and eNAT™ had comparable Cₜs except at lower dilutions eNAT™ had an earlier Cₜ. Therefore, TE buffer results in the latest Cₜ, followed by UTM at day 7.

At day 14 room temperature (table 6), DB #1 was comparable or slightly better than eNAT™ for E Gene and UTR and was comparable or slightly better than UTM at lower concentrations for E Gene. DB #2 was comparable or better than eNAT™ for both UTR and E Gene, and comparable or slightly better than UTM at lower concentrations for E Gene. DB #3 was comparable or slightly better than UTM at lower concentrations and comparable to eNAT™ for E Gene. DB #3 was comparable to eNAT™ at higher concentrations, but at lower concentrations eNAT™ was slightly better. DB #4 showed comparable Cₜ to eNAT™ at higher concentrations. At lower concentrations, eNAT™ was slightly better than DB composition 4. DB #4 showed later Cₜ than UTM for E Gene. DB #5 and 6 are comparable to UTM for E gene. DB #5 and 6 are comparable to eNAT™ or slightly better for both eNAT™ and E gene. TE Buffer had a later Cₜ. UTM/Lysis buffer/eNAT™ were all comparable with samples of higher concentrations for UTR.

At day 14, 4° C. (table 6), DB #1 was comparable to eNAT™ but with higher concentration of samples, Cₜ earlier with DB1. DB #2 comparable or slightly earlier Cₜ than eNAT™. DB #3 generally comparable to eNAT™. DB #3 was comparable to eNAT™ for UTR and was also comparable to eNAT™ for E gene with the exception of 1 replicate at 10⁻⁶ where Cₜ for DB #3 is 37.98 vs 34.48 for eNAT™. DB #4 comparable to eNAT™. DB #5 was comparable or slightly better than eNAT™. DB #6 was comparable to eNAT™ for UTR and comparable to eNAT™ with E gene except at 10⁻⁶ where it was ˜2 cycles slower than eNAT™. eNAT™ and Lysis buffer were comparable for UTR and eNAT™ had a slightly better Cₜ than lysis buffer for E gene at the lower concentrations. Lysis and UTM were comparable with UTR and for E Gene UTM had better Cₜ at higher concentrations but worst Cₜ at lower concentrations. TE buffer had a later Cₜ.

The stability data suggests that the composition of DB #5 (‘McMaster Molecular Medium”) has better performance on average compared to other commercially available molecular transport mediums, including eNAT™. Compared to other non-inactivating mediums, such as UTM/VTM, there is a significant room temperature and 4 degree stability over a 2-week period.

### Example 3. Sample Pooling for High-Throughput Screening

Pooling of 4 dilution factors: To identify whether dilution factor is impacting the Cₜ results, 250 μL of COVID-positive sample was extracted as the unpooled sample (40 μL elution volume). The pooled sample in UTM consisted of 125 μL COVID-positive sample+375 μL UTM (25 μL elution volume). The 20 μL PCR reaction contained 2 μL dH₂O, 10 μL 2×Luna 1-Step Probe Mix, 2 μL 10×Primer Mix (E gene/UTR/RNase P), 1 μL Luna RT Mix and 5 μL Template. Cycling conditions: 60° C.×10 mins, 95° C.×2 mins, 45 cycles—95° C.×10 secs, 60° C.×15 secs.

As demonstrated in Table 7, the pool 4 with UTM does not result in the same Cₜ values as no pool.

The next experiment was completed with COVID positive samples collected in Cobas media rather than UTM, and the elution volumes were increased to reduce the effects of smaller volumes (i.e. small differences more pronounced with smaller volumes). Three conditions were used: A) 250 μL COVID-positive sample (elution volume 80 μL), B) 500 μL COVID-positive sample (elution volume 80 μL), C) 125 μL COVID-positive sample+375 μL Lysis Buffer or UTM (elution volume 40 μL). Condition C mimics the pooling of 4 NPS samples to help evaluate whether there is a dilution factor occurring.

As demonstrated in Table 8 and confirmed in Table 9, conditions A and C (with lysis buffer) are the same, indicating there are no dilution factors. Condition C (with UTM) results in a higher Cₜ score, indicating UTM is having an inhibitory effect, which explains why pools of 500 μL result in worse Cₜ thresholds than pools of 300 μL. The inhibitory effect of UTM is more pronounced in samples of lower concentration.

Pooling of real samples: Unpooled sample: 250 μL of COVID positive sample was extracted (elution volume 70 μL), Pooled sample in Lysis buffer or UTM: 125 μL COVID-positive sample+375 μL Lysis Buffer or UTM (elution volume 35 μL) (Condition C) which mimics the pool of 4 but using Lysis Buffer or UTM instead of negative NPS samples), Real pooled sample (Condition C): 125 μl COVID positive sample+375 μl real clinical sample in Lysis Buffer (i.e. 3 NPS swabs vortexed in 375 μl Lysis Buffer). These swabs were collected in UTM so there is some UTM carried over.

As demonstrated in Table 10, pooling results in lower Cₜ values than no pool. In the unpooled sample, there is more UTM which is potentially causing inhibition. There is not a large difference in the samples diluted with lysis buffer or UTM, likely because the COVID sample is already in UTM. With the NPS samples pooled, the crossing thresholds are comparable.

Pooling compatibility with DB #1-6: Unpooled sample: 250 μl COVID positive sample extracted (elution volume 70 μl), Pooled sample in DB Buffer 1 to 6: 125 μl COVID positive sample (collected in Cobas media)+375 μl DB Buffer 1 to 6; (elution volume 35 μl) which this mimics the pool of 4 but using buffer instead of negative NPS samples. PCR reaction and cycling conditions are the same as described above.

As described in Table 11, DB #1-6 do not cause any interference for pooling, however, DB #1 and 2 yield later crossing thresholds compared to “no pool”.

Pooling of 4 to 8 samples in DB #5: Traditionally with samples collected in transport media, 250 μl is added to 2 ml lysis buffer×10 minutes for lysis. Then magnetic beads are added to lysate and the testing proceeds with easyMAG® extraction. With the DB #5, the cells are already lysed. Therefore, the next experiments were conducted to determine whether the lysis step could be skipped, and whether samples collected in DB #5 could be pooled together to make up 2 mL. NPS samples (2 mL) collected in UTM or Cobas media were spun at 13,000 rpm for 5 mins. Cell pellet was washed 2× with 1 mL PBS to remove the original media and the cell pellet was resuspended in 2 mL of Lysis Buffer (as a point of reference) or DB #5. Serial dilutions of the positive samples were prepared and pooled together with either 3 or 7 clinical COVID negative samples. Magnetic beads were added to “pools” and followed easyMAG® protocol. The eluates were tested by COVID Triplex PCR as described in Example 2.

TABLE 1
Experimental set-up

| | Positive Sample | Negative Sample | Elution |
|---|---|---|---|
| | Volume | Volume | Volume |
| No Pool | 250 μl | 0 | 40 μl |
| Pool 4 | 500 μl | 3 × 500 μl | 80 μl |
| Pool 8 | 250 μl | 7 × 250 μl | 40 μl |

As demonstrated in Table 12, the crossing thresholds for both pool sizes are comparable to the no pool, with accuracy decreasing at the lower concentrations as the limit of detection is reached. Lysis Buffer and DB #5 were comparable, with DB #5 resulting in marginally earlier crossing thresholds.

### Example 4. 270 Day Sample Stability in DB #5

To identify whether nucleic acid samples are stable over time, mocked clinical SARS-CoV-2 samples were spiked into DB #5 and stored for up to 270 days at 4 C, room temperature, and 37 C.

Methods: Mocked clinical SARS-CoV-2 samples were spiked into DB #5. Briefly, a clinical COVID-19-positive nasopharyngeal swab was collected in 1 mL UTM and spun at 13,000 rpm for 5 mins. The cell pellet was washed in phosphate buffered saline (PBS) twice before resuspension in 2 mL DB #5. Samples (250 uL) were extracted at days 0, 7, 14, 30, 60, 90, 120, 150, 180, 210, and 270 via easyMAG® and tested in a real-time reverse transcriptase polymerase chain reaction (RT-PCR) triplex assay targeting E gene, UTR, and RNAse P (as a sample adequacy/control marker) to detect SARS-CoV-2 RNA.

Nucleic acid samples were considered stable if less than a +/−3.0 cycle difference from time zero was observed, as determined by RT-PCR.

Results: As described in Table 13, samples stored at 37 C have a lower level of stability compared to Room Temp (RT) and 4 C. Samples were stable for at least 2 weeks at 37 C. Samples at 4 C and Room Temperature were stable for at least 2 months.

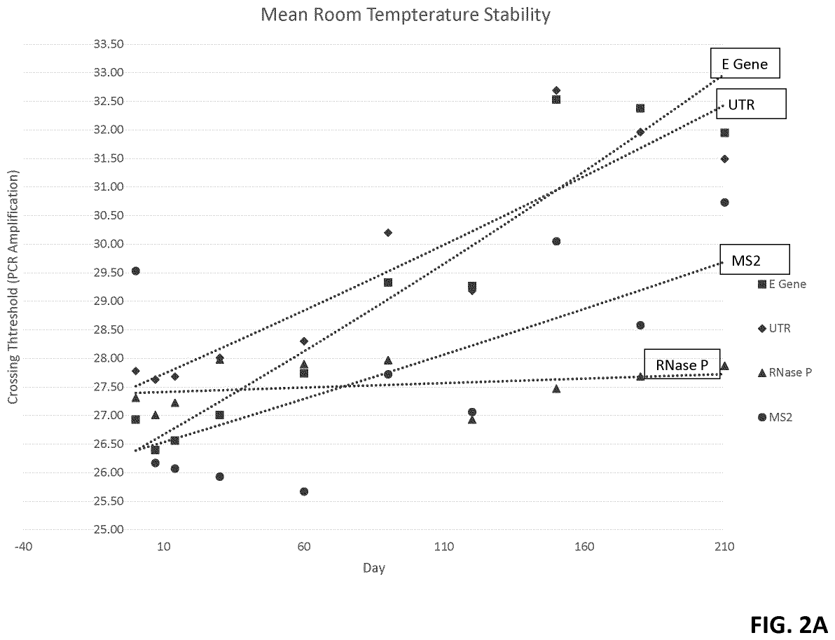

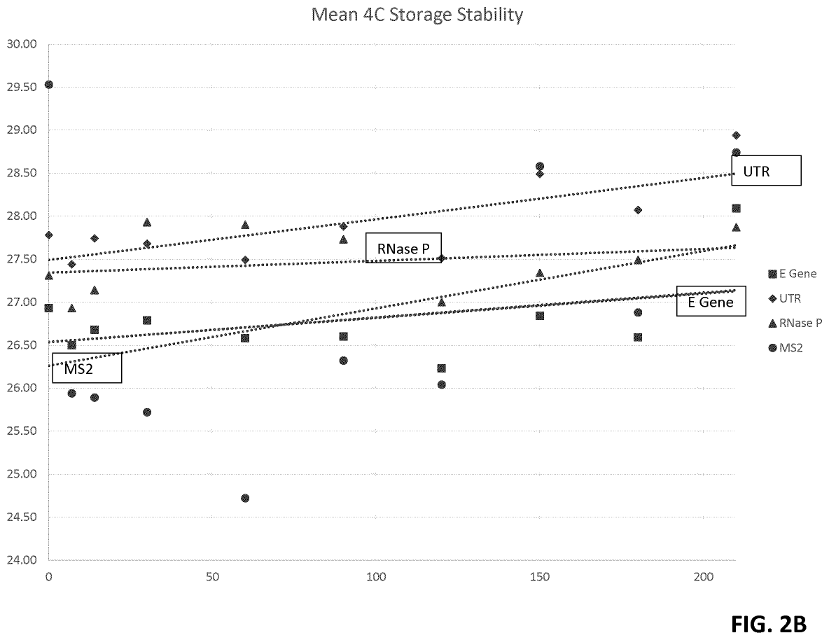

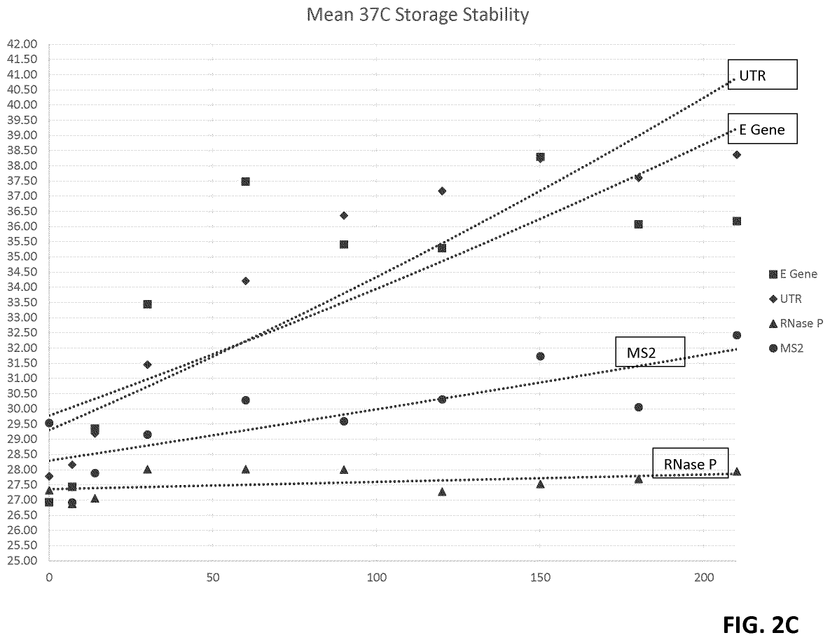

As shown in tables 14, 15, 16, and 17, and FIGS. 2A-C, samples stored at 37 C were stable for at least two weeks, samples stored at Room Temperature were stable for at least 6 months, and samples stored at 4 C were stable for at least 9 months.

### Example 5. Stability of Transport Media

To test the shelf life of the transport media, every batch of DB #5 produced has been compared to the original batch produced.

Methods: A COVID positive patient sample (24193) was spun at 13000 rpm for 5 mins. The cell pellet washed with PBS and resuspended in TE buffer.

Similarly, pooled COVID negative patient samples were spun at 13000 rpm for 5 mins. The cell pellet washed once with PBS and resuspended in TE buffer.

Mocked samples were prepared as follows: A) 900 μl of 24193 10⁻² cell suspension+900 μl negative cell suspension added to 7200 μl MMM (of various lots) to achieve a final concentration of COVID=10⁻³, RNase P 10⁻¹

B) 900 μl of 24193 10⁻⁴ cell suspension+900 μl negative cell suspension added to 7200 μl MMM (of various lots) to achieve a final concentration of COVID=10⁻⁴, RNase P 10⁻¹

Note: To determine whether Cobas swabs could be used with DB #5, Cobas NPS or oral swabs were added to 1 set of mocked MMM samples.

Mocked samples were divided into 2 ml aliquots. 2×2 ml aliquots were extracted via easyMAG immediately (i.e. 50 μl silica added, washes etc); elution volume=50 μl

2×2 ml aliquots were left at room temperature for 72 hours and then extracted via easyMAG (i.e. 50 μl silica added, washes etc); elution volume=50 μl

Eluates were tested in COVID triplex PCR using standard protocols. A typical experimental setup is shown below:

| | |
|---|---|
| dH₂0 | 2 |
| Luna ™ 1-Step Probe RT Master Mix | 10 |
| 10 x Primer Mix | 2 |
| RT Enzyme | 1 |
| RNA Template | 5 |
| TOTAL | 20 μl |

Results: As described in Table 18, DB #5 is performing equally as well at 112 days (i.e. about 4 months) as fresh media.

As described in Table 19, DB #5 is performing equally well at about 6 months as fresh media.

### Example 6. Performance of DB #5 with and without Bovine Serum Albumin (BSA) or Dithiothreitol (DTT)

To test the performance and stability of DB #5 relative to media comprising a reducing/mucolytic agent, or BSA, formulations of DB #5 comprising different amounts of DTT or BSA were tested.

Methods: DB #5 was formulated with the following additives:
DB #5 (Normal)
DB #5+0.05 mg/mL BSA
DB #5+0.1 mg/mL BSA
DB #5+0.25 mM DTT (reducing/mucolytic agent)
DB #5+1 mM DTT (reducing/mucolytic agent)

The transport media variants were spiked with equal amounts of MS2 (RNA Control Phage) and SARS-CoV-2 patient sample and then stored at room temperature or 4 C. Samples were extracted on Day 0, Day 3 and Day 7.

Results: As described in Table 20, no difference in nucleic acid stability was noted in DB #5 with or without the addition of reducing/mucolytic agent or with or without the addition of BSA.

### Example 7. Lysis of Samples

Background & Objective: The RNA extraction protocol (Promega extraction on a Hamilton robot) calls for a sample volume, an equal volume of lysis buffer and the sample+lysis buffer volume of isopropanol. For example, for a 250 μl sample: mix together 250 μl sample+250 μl lysis buffer+500 μl isopropanol. This does not take into account bead volume. Higher sample volume increases the risk of cross contamination in automated protocols as the mix must be repeatedly pipetted or vigorously mixed for the nucleic acid to bind to the silica beads. To optimize the extraction protocol to accommodate the Automated robotic extraction and possible pooling scenarios, several factors were assessed: bead volume, binding time, mixing speed, and omitting the lysis buffer.

Bead Volume Titration

Methods: 250 μl sample (dilution of a strong COVID positive in DB #5) was mixed with 250 μl lysis buffer and incubated at 56° C.×10 mins with constant shaking. To the sample was added: 500 μl isopropanol+5, 17.5 or 35 μl beads. Samples+beads were mixed at 1500 rpm for 15 mins and beads were then washed with: 900 μl 4/40 wash solution (Promega catalogue number A2221), 450 μl Alcohol Wash (Promega catalogue number MD1311), and 450 μl 80% ethanol. Residual ethanol was removed and tube of beads “air dried” for 5 mins (i.e tube left open on the grid of the BSC hood). Beads were resuspended with 40 μl water to elute the RNA. On the magnet, beads were pulled to the side of the tube and the eluate transferred to a new tube.

Results: As described in Table 21, the crossing thresholds are increased when a smaller volume of beads is used.

Binding Time Titration and Increased Mixing Speed

Methods: 250 μl sample (dilution of a strong COVID positive in DB #5) was mixed with 250 μl lysis buffer and incubated at 56° C.×10 mins with constant shaking. To the sample was added 500 μl isopropanol+5, 17.5 or 35 μl beads. Samples+beads were mixed at 2000 rpm for 15, 22 or 30 mins and beads were then washed with: 900 μl 4/40 wash solution, 450 μl Alcohol Wash, and 450 μl 80% ethanol. Residual ethanol was removed and tube of beads “air dried” for 5 mins (i.e tube left open on the grid of the BSC hood). Beads were resuspended with 40 μl water to elute the RNA. On the magnet, beads were pulled to the side of the tube and the eluate transferred to a new tube.

Results: As described in Table 22, binding time had no appreciable effect on crossing threshold. With a higher mixing speed (2000 rpm compared to 1500 rpm used to obtain the results in Table 21) the crossing thresholds are comparable when using 17.5 μl versus 35 μl beads, whereas there is an approximate two cycle increase observed when using 5 μl versus 35 μl beads.

Omitting Lysis Buffer

To test whether samples collected in DB #5 require lysis prior to nucleic acid extraction, the following experiments were performed.

Methods: 250 μl sample (dilution of a strong COVID positive in MMM) was incubated at 56° C.×10 mins with constant shaking. To the sample was added 250 μl isopropanol+5, 17.5 or 35 μl beads. Samples+beads were mixed at 2000 rpm for 15 mins and beads were then washed with: 900 μl 4/40 wash solution, 450 μl Alcohol Wash, and 450 μl 80% ethanol. Residual ethanol was removed and tube of beads “air dried” for 5 mins (i.e tube left open on the grid of the BSC hood). Beads were resuspended with 40 μl water to elute the RNA. On the magnet, beads were pulled to the side of the tube and the eluate transferred to a new tube.

Results: As described in Table 23, comparable performance was seen using when lysis buffer was omitted (and the volume of isopropanol was adjusted accordingly). Results using 17.5 μl versus 35 μl beads are comparable.

The formulation of DB #5 facilitates higher throughput and cost savings within the laboratory, as samples collected and stored in DB #5 do not require a separate lysis step prior to nucleic acid extraction, saving time and materials, without loss of performance. The omission of additional lysis buffer results in lower total volumes, which facilitates sample pooling of larger sample numbers while maintaining extraction performance.

### Example 8. Inactivation of SARS-CoV-2

To assess the suitability of DB #5 for direct-from-tube sampling and extraction systems to test for coronavirus, the following experiments were performed to demonstrate that this virus is inactivated by DB #5.

Methods: 100 ul of SARS CoV2 virus stock (Titer 10\hat{ }6.8 TCID50) was added to 400 ul of DMEM media. Then the media with the virus was added to 2 ml of DB #5 lysis buffer, mixed well, and allowed to sit for a minimum of 10 minutes at room temperature. 500 ul of the mix was extracted following the kit procedure for RNA extraction (QIAamp kit from Qiagen, Cat No./ID: 52906.). The RNA extraction was done with duplicate samples. The RNA was eluted with 50 ul of AVE buffer (Qiagen).

Once the RNA extraction was done the 50 ul of eluted RNA was added to Vero cells and incubated for 5 days. In the absences of cytopathic effect (CPE) on day 5 the supernatant of the Vero cells was passaged onto fresh cells and observed for CPE (both original and new plate) until day 14 dpi.

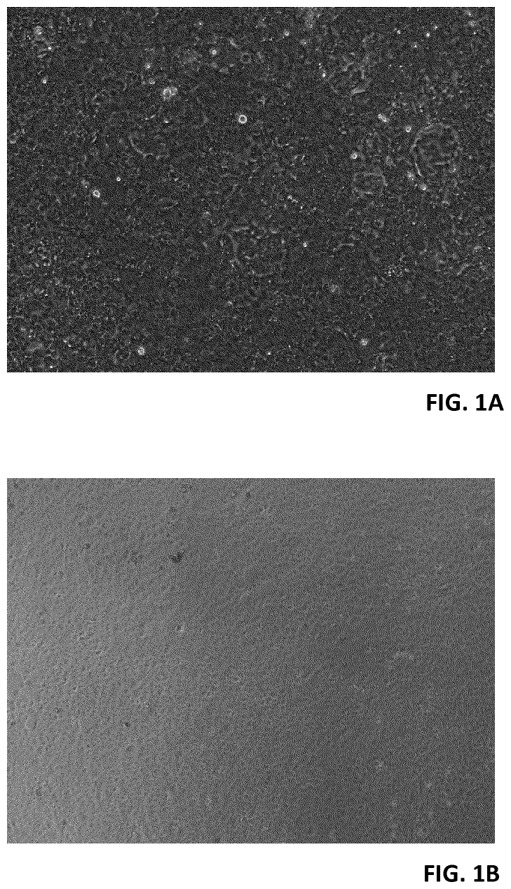

Results: As shown in FIG. 1, no signs of cytopathic effect were observed after 5 days (FIG. 1A) or after 14 days (FIG. 1B), indicating that SARS-CoV-2 was inactivated by incubation with DB #5 for 15 minutes. DB #5 is suitable for direct-from-tube sampling and extraction following as little as 15 minutes incubation in DB #5. There is no need to pre-aliquot samples in a biosafety hood since samples are inactivated.

### Example 9. DB #5 Improves Performance of easyMag Extraction

DB #5 can also be used as a lysis/nucleic acid binding buffer.

To determine whether DB #5 can be used as a lysis/nucleic acid binding buffer, experiments were performed to compare DB #5 to easyMag lysis buffer.

Methods: SARS-CoV-2 samples were spiked into DB #5 or easyMag lysis buffer, and then extracted with easyMag reagents per the manufacturer recommendations with slight modifications. 250 uL of sample was spiked into 2 mL of DB #5 or 2 mL of easyMag lysis buffer. Experiments were performed in triplicate. 50 uL of easyMag silica was added to each tube and incubated at room temperature for 10 minutes. The silica was then washed with easyMag Buffer #1, easyMag Buffer #2, and then washed and eluted in easyMag Buffer #3. Extraction performance was then assessed by qPCR.

Results: As demonstrated in Table 24, DB #5 outperforms easyMag lysis buffer when used as a lysis buffer (i.e. in place of easyMag lysis buffer) prior to extraction with easyMag reagents.

### Example 10. Performance of DB #5 Variants

Three variations of DB #5 were compared to the standard DB #5 at day 0, 1, 3, 5, and 7 at 3 storage temperatures with 4 replicates.

Methods: Mocked clinical SARS-CoV-2 samples were spiked into DB #5 variants. Briefly, a clinical COVID-19-positive nasopharyngeal swab was collected in UTM and spun at 13,000 rpm for 5 mins. The cell pellet was washed in phosphate buffered saline (PBS) twice before resuspension in DB #5 Variants. Samples (250 uL) were extracted at days 0, 1, 3, 5, and 7 via easyMAG® and tested in a real-time reverse transcriptase polymerase chain reaction (RT-PCR) assay targeting E gene and UTR to detect SARS-CoV-2 RNA.

| | Variant #1 | Variant #2 | Variant #3 | DB#5 |
|---|---|---|---|---|
| Component | (MMM#1) | (MMM#2) | (MMM#3) | (MMM#4) |
| Guanidine Thiocyanate | 2.0M | 2.0M | 3.0M | 4.0M |
| Triton X-100 | 1.0% | 2.0% | 2.0% | 2.0% |
| EDTA | 0.020M | 0.040M | 0.040M | 0.040M |
| TRIS-HCl | 0.025M | 0.050M | 0.050M | 0.050M |
| Isopropanol | 10% | 20% | 20% | 20% |

pH = ~7.0 @ 20° C.

Results: the 7 day stability of samples stored in DB #5/MMM variants with lower guanidine thiocyanate concentrations is comparable to or improved over DB #5. As shown in Table 25, the crossing threshold for samples stored in any of DB #5 or variants 1-3 for up to 7 days at 4 C or at room temperature is comparable to day 0. The crossing threshold of samples stored up to 7 days in variants #1 and #2 (MMM #1 and MMM #2) at 37 C is comparable to day 0. The stability of samples stored in variant #3 (MMM #3) or DB #5 (MMM #4) starts to decrease by day 5.

### Example 11. Storage and Detection of Influenza A, SARS-CoV-2, and Group A Streptococcus (GAS)

Standard DB #5 was spiked with Influenza A (Flu A), COVID, or GAS and compared to UTM spiked with Flu A, COVID, or GAS.

Methods: Mocked Flu A and COVID samples were prepared essentially as described above in Example 10. Briefly, Flu A NPS clinical positives and COVID NPS clinical positives were collected in 1 mL UTM and spun at 13,000 rpm for 5 mins. The cell pellet was washed in phosphate buffered saline (PBS) twice before resuspension in DB #5. Samples (250 uL) were extracted at days 0, 1, 3, 5, and 7 via easyMAG® and tested in a real-time reverse transcriptase polymerase chain reaction (RT-PCR). Flu A and COVID samples were pooled for combined testing. For combined FluA+COVID samples, the PCR used for testing was a pentaplex targeting 5 targets (Flu A, Flu B, RSV, COVID (both the E gene and UTR combined in 1 channel) and RNase P that was designed in-house. For COVID samples, PCR testing was carried out using a triplex assay targeting E gene, UTR, and RNAse P.

Mocked GAS samples were obtained from plated colonies that were swiped onto a swab. Separate samples were prepared in MMM and UTM. Samples were extracted at days 0, 1, 3, 5, and 7 via easyMAG® and tested in a real-time reverse transcriptase polymerase chain reaction (RT-PCR). The PCR used for testing is a lab-developed test targeting the DNase B gene.

Results: MMM performs equally well for the storage and detection of Influenza A, SARS-CoV-2, and GAS, as compared to standard UTM. As shown in Tables 26 and 27, the crossing thresholds of Flu A and COVID targets (Table 26) or COVID targets (Table 27) of samples stored up to 7 days in MMM or UTM at 4 C, room temperature, or 37 C are comparable to day 0. As shown in Table 26, RNase P stability decreases in both MMM and UTM regardless of the storage temperature, although other targets are comparable to Day 0. A similar trend is seen in Table 27, samples stored up to 7 days in MMM or UTM at 4 C or room temperature show comparable CT values to Day 0. Samples stored in MMM at 37° C. for up to 7 days are comparable as well. Samples stored in UTM for up to 7 days show E gene/UTR CT values that are comparable to day 0, but the RNase P CT value increases by 3.35.

As shown in Table 28, GAS samples stored in MMM are stable for at least 7 days regardless of the storage temperature, whereas samples stored in UTM have increased CT values when stored at 37 C and/or over time.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 2
Sample stability data at day 0 of DB#1-6
compared to other transport media.

| | | | E Gene | E Gene | UTR | UTR |
|---|---|---|---|---|---|---|
| | Day | | at RT | at RT | at RT | at RT |
| Buffer | # | Dil | Rep 1 | Repl 2 | Rep 1 | Rep 2 |
| UTM | 0 | 10⁻³ | 25.20 | 25.06 | 26.26 | 26.02 |
| UTM | 0 | 10⁻⁴ | 27.99 | 27.98 | 28.66 | 28.74 |
| UTM | 0 | 10⁻⁵ | 31.25 | 30.77 | 31.84 | 31.50 |
| UTM | 0 | 10⁻⁶ | 35.17 | 36.21 | 35.72 | 36.09 |
| TE Buffer | 0 | 10⁻³ | 25.17 | 25.15 | 25.67 | 25.50 |
| TE Buffer | 0 | 10⁻⁴ | 27.80 | 28.04 | 29.12 | 29.36 |
| TE Buffer | 0 | 10⁻⁵ | 32.27 | 32.17 | 32.62 | 32.73 |
| TE Buffer | 0 | 10⁻⁶ | 35.72 | 35.47 | 35.41 | 35.67 |
| Lysis Buffer | 0 | 10⁻³ | 24.82 | 24.95 | 25.29 | 25.38 |
| Lysis Buffer | 0 | 10⁻⁴ | 27.93 | 28.09 | 28.36 | 28.45 |
| Lysis Buffer | 0 | 10⁻⁵ | 30.98 | 31.31 | 31.38 | 31.76 |
| Lysis Buffer | 0 | 10⁻⁶ | 34.35 | 33.62 | 34.21 | 34.48 |
| eNAT ™ | 0 | 10⁻³ | 24.93 | 24.92 | 25.14 | 25.12 |
| eNAT ™ | 0 | 10⁻⁴ | 27.65 | 28.02 | 28.07 | 28.43 |
| eNAT ™ | 0 | 10⁻⁵ | 31.13 | 31.32 | 31.48 | 31.72 |
| eNAT ™ | 0 | 10⁻⁶ | 34.56 | 34.91 | 34.65 | 34.77 |
| DB Buffer 1 | 0 | 10⁻³ | 24.95 | 24.92 | 25.48 | 25.56 |
| DB Buffer 1 | 0 | 10⁻⁴ | 28.06 | 28.07 | 28.61 | 28.58 |
| DB Buffer 1 | 0 | 10⁻⁵ | 30.52 | 30.67 | 31.05 | 31.08 |
| DB Buffer 1 | 0 | 10⁻⁶ | 33.68 | 33.92 | 34.22 | 34.55 |
| DB Buffer 2 | 0 | 10⁻³ | 24.64 | 25.01 | 25.14 | 25.31 |
| DB Buffer 2 | 0 | 10⁻⁴ | 27.36 | 27.82 | 27.94 | 28.32 |
| DB Buffer 2 | 0 | 10⁻⁵ | 30.95 | 31.07 | 31.35 | 31.58 |
| DB Buffer 2 | 0 | 10⁻⁶ | 33.56 | 34.00 | 34.56 | 34.84 |
| DB Buffer 3 | 0 | 10⁻³ | 24.93 | 25.05 | 25.28 | 25.39 |
| DB Buffer 3 | 0 | 10⁻⁴ | 27.92 | 28.15 | 28.26 | 28.51 |
| DB Buffer 3 | 0 | 10⁻⁵ | 30.86 | 31.19 | 31.05 | 31.35 |
| DB Buffer 3 | 0 | 10⁻⁶ | 34.39 | 35.78 | 34.35 | 35.33 |
| DB Buffer 4 | 0 | 10⁻³ | 24.61 | 24.95 | 24.88 | 25.19 |
| DB Buffer 4 | 0 | 10⁻⁴ | 27.36 | 27.71 | 27.83 | 28.10 |
| DB Buffer 4 | 0 | 10⁻⁵ | 30.73 | 31.03 | 31.12 | 31.21 |
| DB Buffer 4 | 0 | 10⁻⁶ | 34.06 | 33.88 | 34.15 | 34.18 |
| DB Buffer 5 | 0 | 10⁻³ | 24.80 | 25.02 | 25.06 | 25.43 |
| DB Buffer 5 | 0 | 10⁻⁴ | 27.51 | 28.01 | 28.07 | 28.38 |
| DB Buffer 5 | 0 | 10⁻⁵ | 30.98 | 31.62 | 31.20 | 31.80 |
| DB Buffer 5 | 0 | 10⁻⁶ | 34.06 | 34.48 | 34.21 | 34.78 |
| DB Buffer 6 | 0 | 10⁻³ | 24.42 | 24.98 | 24.74 | 25.27 |
| DB Buffer 6 | 0 | 10⁻⁴ | 27.33 | 28.08 | 27.63 | 28.32 |
| DB Buffer 6 | 0 | 10⁻⁵ | 30.63 | 31.30 | 31.08 | 31.30 |
| DB Buffer 6 | 0 | 10⁻⁶ | 34.43 | 34.30 | 34.39 | 35.14 |

Abbreviations: Dil, Dilution; Rep, Replicate;

TABLE 3
Sample stability data at day 1 after storage at room temperature (RT)
or at 4° C. of DB#1-6 compared to other transport media.

| | | | E Gene | E Gene | UTR | UTR | E Gene | E gene | UTR | UTR |
|---|---|---|---|---|---|---|---|---|---|---|
| | Day | | at RT | at RT | at RT | at RT | at 4 C. | at 4 C. | at 4 C. | at 4 C. |
| Buffer | # | Dil | Rep 1 | Rep 2 | Rep 1 | Rep 2 | Rep 1 | Rep 2 | Rep 1 | Rep 2 |
| UTM | 1 | 10⁻³ | 25.51 | 25.45 | 26.56 | 26.43 | 24.00 | 23.74 | 24.63 | 24.41 |
| UTM | 1 | 10⁻⁴ | 28.48 | 28.34 | 29.37 | 29.26 | 27.25 | 27.32 | 27.53 | 27.81 |
| UTM | 1 | 10⁻⁵ | 32.03 | 32.48 | 32.98 | 32.94 | 30.38 | 30.67 | 30.81 | 31.03 |
| UTM | 1 | 10⁻⁶ | 35.42 | 35.45 | 35.62 | 35.27 | 35.60 | 35.02 | 36.53 | 36.92 |
| TE Buffer | 1 | 10⁻³ | 26.10 | 25.89 | 26.81 | 26.53 | 26.44 | 25.67 | 26.90 | 26.17 |
| TE Buffer | 1 | 10⁻⁴ | 29.11 | 29.20 | 29.77 | 29.79 | 29.09 | 29.21 | 29.32 | 29.35 |
| TE Buffer | 1 | 10⁻⁵ | 33.25 | 32.92 | 33.56 | 33.47 | 32.60 | 32.41 | 32.81 | 32.64 |
| TE Buffer | 1 | 10⁻⁶ | 35.80 | 36.06 | 35.44 | 35.38 | 37.45 | 38.13 | 36.48 | 36.44 |
| Lysis Buffer | 1 | 10⁻³ | 23.82 | 23.72 | 24.31 | 24.26 | 24.02 | 23.81 | 24.37 | 24.14 |
| Lysis Buffer | 1 | 10⁻⁴ | 27.16 | 27.18 | 27.54 | 27.79 | 27.25 | 27.25 | 27.38 | 27.50 |
| Lysis Buffer | 1 | 10⁻⁵ | 30.75 | 30.48 | 30.97 | 31.00 | 30.68 | 31.11 | 31.12 | 31.02 |
| Lysis Buffer | 1 | 10⁻⁶ | 34.79 | 34.35 | 35.16 | 34.61 | 34.91 | 34.41 | 34.32 | 34.68 |
| eNAT ™ | 1 | 10⁻³ | 23.66 | 23.53 | 24.18 | 24.06 | 23.70 | 23.70 | 24.03 | 24.01 |
| eNAT ™ | 1 | 10⁻⁴ | 27.69 | 27.58 | 28.20 | 28.19 | 27.25 | 27.08 | 27.64 | 27.31 |
| eNAT ™ | 1 | 10⁻⁵ | 30.31 | 30.30 | 30.89 | 30.69 | 30.32 | 30.22 | 30.81 | 30.50 |
| eNAT ™ | 1 | 10⁻⁶ | 34.03 | 33.40 | 34.23 | 33.83 | 33.56 | 34.07 | 34.16 | 34.27 |
| DB Buffer 1 | 1 | 10⁻³ | 23.94 | 23.78 | 24.49 | 24.34 | 24.23 | 24.08 | 24.93 | 24.56 |
| DB Buffer 1 | 1 | 10⁻⁴ | 27.02 | 27.02 | 27.48 | 27.55 | 27.18 | 27.17 | 27.50 | 27.62 |
| DB Buffer 1 | 1 | 10⁻⁵ | 30.22 | 30.17 | 30.60 | 30.43 | 30.46 | 30.53 | 30.68 | 30.95 |
| DB Buffer 1 | 1 | 10⁻⁶ | 33.25 | 33.70 | 33.85 | 34.00 | 33.94 | 33.55 | 34.60 | 34.35 |
| DB Buffer 2 | 1 | 10⁻³ | 24.04 | 23.75 | 24.56 | 24.28 | 24.03 | 23.75 | 24.60 | 24.28 |
| DB Buffer 2 | 1 | 10⁻⁴ | 26.82 | 26.56 | 27.40 | 27.21 | 26.96 | 26.98 | 27.33 | 27.42 |
| DB Buffer 2 | 1 | 10⁻⁵ | 30.34 | 30.55 | 31.03 | 30.90 | 30.36 | 30.29 | 30.74 | 30.67 |
| DB Buffer 2 | 1 | 10⁻⁶ | 34.10 | 34.09 | 34.19 | 33.93 | 33.29 | 34.27 | 34.28 | 34.47 |
| DB Buffer 3 | 1 | 10⁻³ | 23.62 | 23.49 | 24.14 | 24.02 | 23.85 | 23.73 | 24.37 | 24.20 |
| DB Buffer 3 | 1 | 10⁻⁴ | 26.76 | 26.67 | 27.09 | 27.10 | 26.84 | 26.82 | 27.06 | 27.05 |
| DB Buffer 3 | 1 | 10⁻⁵ | 30.10 | 29.98 | 30.71 | 30.30 | 30.25 | 30.12 | 30.35 | 30.33 |
| DB Buffer 3 | 1 | 10⁻⁶ | NA | NA | NA | NA | 33.65 | 34.19 | 33.89 | 34.22 |
| DB Buffer 4 | 1 | 10⁻³ | 23.82 | 23.63 | 24.19 | 23.97 | 23.80 | 23.38 | 24.22 | 23.66 |
| DB Buffer 4 | 1 | 10⁻⁴ | 26.87 | 26.78 | 27.15 | 27.15 | 27.03 | 26.95 | 27.19 | 27.09 |
| DB Buffer 4 | 1 | 10⁻⁵ | 30.35 | 30.24 | 30.77 | 30.71 | 30.27 | 30.43 | 30.40 | 30.41 |
| DB Buffer 4 | 1 | 10⁻⁶ | 33.45 | 33.32 | 33.43 | 33.57 | 34.28 | 34.14 | 33.99 | 34.32 |
| DB Buffer 5 | 1 | 10⁻³ | 23.49 | 23.41 | 23.93 | 23.88 | 24.07 | 23.84 | 23.97 | 23.77 |
| DB Buffer 5 | 1 | 10⁻⁴ | 28.16 | 28.13 | 28.42 | 28.42 | 27.18 | 27.20 | 27.07 | 27.09 |
| DB Buffer 5 | 1 | 10⁻⁵ | 30.39 | 30.45 | 30.48 | 30.42 | 30.54 | 30.49 | 30.55 | 30.63 |
| DB Buffer 5 | 1 | 10⁻⁶ | 33.32 | 33.76 | 33.51 | 33.70 | 34.16 | 34.27 | 33.86 | 34.26 |
| DB Buffer 6 | 1 | 10⁻³ | 23.32 | 23.26 | 23.69 | 23.71 | 23.60 | 23.37 | 24.04 | 23.66 |
| DB Buffer 6 | 1 | 10⁻⁴ | 26.94 | 26.89 | 27.07 | 27.10 | 27.11 | 27.16 | 27.20 | 27.20 |
| DB Buffer 6 | 1 | 10⁻⁵ | 30.18 | 30.10 | 30.29 | 30.26 | 30.19 | 30.46 | 30.63 | 30.56 |
| DB Buffer 6 | 1 | 10⁻⁶ | 33.47 | 33.57 | 33.73 | 34.05 | 33.62 | 34.08 | 34.12 | 33.94 |

Abbreviations: Dil, Dilution; Rep, Replicate.

TABLE 4
Sample stability data at day 3 after storage at room temperature (RT)
or at 4° C. of DB#1-6 compared to other transport media.

| | | | E Gene | E Gene | UTR | UTR | E Gene | E gene | UTR | UTR |
|---|---|---|---|---|---|---|---|---|---|---|
| | Day | | at RT | at RT | at RT | at RT | at 4 C. | at 4 C. | at 4 C. | at 4 C. |
| Buffer | # | Dil | Rep 1 | Rep 2 | Rep 1 | Rep 2 | Rep 1 | Rep 2 | Rep 1 | Rep 2 |
| UTM | 3 | 10⁻³ | 25.48 | 25.34 | 26.47 | 26.39 | 24.34 | 24.07 | 25.06 | 24.64 |
| UTM | 3 | 10⁻⁴ | 27.21 | 27.25 | 28.15 | 28.17 | 27.40 | 27.37 | 27.67 | 27.69 |
| UTM | 3 | 10⁻⁵ | 32.20 | 32.06 | 32.63 | 32.79 | 30.29 | 30.29 | 30.25 | 30.22 |
| UTM | 3 | 10⁻⁶ | 36.30 | 35.62 | 36.66 | 36.87 | 35.34 | 35.02 | 36.18 | 36.81 |
| TE Buffer | 3 | 10⁻³ | 26.58 | 26.14 | 27.47 | 27.04 | 26.99 | 26.13 | 27.67 | 26.62 |
| TE Buffer | 3 | 10⁻⁴ | 29.55 | 29.45 | 30.21 | 30.16 | 29.25 | 29.31 | 29.63 | 29.88 |
| TE Buffer | 3 | 10⁻⁵ | 32.43 | 32.81 | 33.24 | 32.56 | 33.54 | 32.84 | 33.01 | 32.69 |
| TE Buffer | 3 | 10⁻⁶ | 35.13 | 35.50 | 35.28 | 35.11 | 33.62 | 34.05 | 33.25 | 33.11 |
| Lysis Buffer | 3 | 10⁻³ | 23.96 | 23.76 | 24.44 | 24.20 | 24.04 | 23.83 | 24.47 | 24.18 |
| Lysis Buffer | 3 | 10⁻⁴ | 27.14 | 27.10 | 27.42 | 27.47 | 27.27 | 27.19 | 27.63 | 27.52 |
| Lysis Buffer | 3 | 10⁻⁵ | 31.10 | 30.99 | 31.26 | 31.04 | 31.37 | 31.23 | 31.43 | 31.49 |
| Lysis Buffer | 3 | 10⁻⁶ | 37.80 | 37.53 | 35.27 | 35.53 | 34.23 | 34.81 | 34.73 | 34.76 |
| eNAT ™ | 3 | 10⁻³ | 24.00 | 23.57 | 24.45 | 24.08 | 24.10 | 23.83 | 24.53 | 24.10 |
| eNAT ™ | 3 | 10⁻⁴ | 27.27 | 27.17 | 27.50 | 27.48 | 27.12 | 27.15 | 27.28 | 27.37 |
| eNAT ™ | 3 | 10⁻⁵ | 30.48 | 30.29 | 30.75 | 30.57 | 30.62 | 30.48 | 30.83 | 30.68 |
| eNAT ™ | 3 | 10⁻⁶ | 34.43 | 33.88 | 34.92 | 34.34 | 34.26 | 34.08 | 33.98 | 34.20 |
| DB Buffer 1 | 3 | 10⁻³ | 23.97 | 23.74 | 24.54 | 24.20 | 24.13 | 23.82 | 24.72 | 24.40 |
| DB Buffer 1 | 3 | 10⁻⁴ | 26.85 | 26.83 | 27.26 | 27.26 | 27.24 | 27.18 | 27.76 | 27.69 |
| DB Buffer 1 | 3 | 10⁻⁵ | 30.35 | 30.52 | 31.07 | 31.18 | 30.15 | 30.19 | 30.48 | 30.78 |
| DB Buffer 1 | 3 | 10⁻⁶ | 33.41 | 33.15 | 34.35 | 34.50 | 33.34 | 33.56 | 34.20 | 34.27 |
| DB Buffer 2 | 3 | 10⁻³ | 24.01 | 23.79 | 24.64 | 24.37 | 24.09 | 23.84 | 24.80 | 24.54 |
| DB Buffer 2 | 3 | 10⁻⁴ | 28.35 | 28.18 | 29.10 | 28.94 | NA | NA | NA | NA |
| DB Buffer 2 | 3 | 10⁻⁵ | 30.18 | 30.05 | 30.65 | 30.40 | 30.19 | 29.97 | 30.37 | 30.29 |
| DB Buffer 2 | 3 | 10⁻⁶ | 33.51 | 33.50 | 34.42 | 34.00 | 34.02 | 35.04 | 34.08 | 34.24 |
| DB Buffer 3 | 3 | 10⁻³ | 24.05 | 23.85 | 24.41 | 24.24 | 23.87 | 23.63 | 24.21 | 23.99 |
| DB Buffer 3 | 3 | 10⁻⁴ | 27.30 | 27.18 | 24.68 | 27.65 | 26.89 | 26.79 | 27.13 | 27.07 |
| DB Buffer 3 | 3 | 10⁻⁵ | 30.61 | 30.56 | 31.19 | 31.01 | 30.13 | 29.97 | 30.12 | 29.93 |
| DB Buffer 3 | 3 | 10⁻⁶ | 33.40 | 33.92 | 34.27 | 34.29 | 34.02 | 33.50 | 33.43 | 33.64 |
| DB Buffer 4 | 3 | 10⁻³ | 24.09 | 23.75 | 24.41 | 24.04 | 23.78 | 23.34 | 24.09 | 23.53 |
| DB Buffer 4 | 3 | 10⁻⁴ | 27.34 | 27.21 | 27.60 | 27.48 | NA | NA | NA | NA |
| DB Buffer 4 | 3 | 10⁻⁵ | 30.68 | 30.63 | 31.09 | 30.85 | 30.03 | 30.17 | 30.24 | 30.24 |
| DB Buffer 4 | 3 | 10⁻⁶ | 33.63 | 33.62 | 33.99 | 34.06 | 33.45 | 33.61 | 33.50 | 33.74 |
| DB Buffer 5 | 3 | 10⁻³ | 23.68 | 23.50 | 24.03 | 23.77 | 24.00 | 23.73 | 24.23 | 24.12 |
| DB Buffer 5 | 3 | 10⁻⁴ | 26.96 | 26.89 | 27.03 | 26.95 | 26.96 | 26.90 | 27.18 | 27.15 |
| DB Buffer 5 | 3 | 10⁻⁵ | 31.48 | 31.23 | 31.20 | 31.15 | 30.48 | 30.38 | 30.50 | 30.38 |
| DB Buffer 5 | 3 | 10⁻⁶ | 33.84 | 33.73 | 34.02 | 33.53 | 34.09 | 33.66 | 33.66 | 33.71 |
| DB Buffer 6 | 3 | 10⁻³ | 23.46 | 23.32 | 23.84 | 23.65 | 24.08 | 23.76 | 24.50 | 24.10 |
| DB Buffer 6 | 3 | 10⁻⁴ | 27.04 | 26.87 | 27.06 | 26.94 | 27.15 | 27.07 | 27.22 | 27.26 |
| DB Buffer 6 | 3 | 10⁻⁵ | 30.41 | 30.42 | 30.42 | 30.20 | 30.44 | 30.37 | 30.69 | 30.70 |
| DB Buffer 6 | 3 | 10⁻⁶ | 34.08 | 33.86 | 33.27 | 33.54 | 34.30 | 34.08 | 33.63 | 33.99 |

Abbreviations: Dil, Dilution; Rep, Replicate.

TABLE 5
Sample stability data at day 7 after storage at room temperature (RT)
or at 4° C. of DB#1-6 compared to other transport media.

| | | | E Gene | E Gene | UTR | UTR | E Gene | E gene | UTR | UTR |
|---|---|---|---|---|---|---|---|---|---|---|
| | Day | | at RT | at RT | at RT | at RT | at 4 C. | at 4 C. | at 4 C. | at 4 C. |
| Buffer | # | Dil | Rep 1 | Rep 2 | Rep 1 | Rep 2 | Rep 1 | Rep 2 | Rep 1 | Rep 2 |
| UTM | 7 | 10⁻³ | 26.00 | 25.90 | 27.35 | 27.28 | 25.45 | 25.21 | 26.35 | 26.02 |
| UTM | 7 | 10⁻⁴ | 28.07 | 28.04 | 28.59 | 28.85 | 28.47 | 28.58 | 29.15 | 29.23 |
| UTM | 7 | 10⁻⁵ | 32.14 | 32.20 | 33.43 | 33.60 | 31.67 | 31.57 | 31.17 | 31.94 |
| UTM | 7 | 10⁻⁶ | 36.99 | 36.18 | 36.98 | 37.65 | 35.39 | 35.80 | 35.53 | 36.41 |
| TE Buffer | 7 | 10⁻³ | 26.62 | 26.46 | 27.64 | 27.31 | 26.99 | 26.33 | 27.42 | 26.69 |
| TE Buffer | 7 | 10⁻⁴ | 30.03 | 29.93 | 30.46 | 30.59 | 30.35 | 30.23 | 30.74 | 30.91 |
| TE Buffer | 7 | 10⁻⁵ | 33.17 | 33.11 | 33.10 | 33.17 | 33.15 | 33.01 | 32.73 | 32.40 |
| TE Buffer | 7 | 10⁻⁶ | 38.20 | 35.78 | 35.21 | 35.57 | 38.68 | 38.85 | 39.06 | 38.12 |
| Lysis Buffer | 7 | 10⁻³ | 23.89 | 23.75 | 24.43 | 24.30 | 24.10 | 23.94 | 24.38 | 24.12 |
| Lysis Buffer | 7 | 10⁻⁴ | 27.40 | 27.37 | 27.88 | 27.72 | 27.24 | 27.26 | 27.51 | 27.46 |
| Lysis Buffer | 7 | 10⁻⁵ | 31.59 | 31.45 | 31.46 | 31.30 | 30.86 | 30.62 | 31.01 | 30.99 |
| Lysis Buffer | 7 | 10⁻⁶ | 36.18 | 37.11 | 35.20 | 35.43 | 35.10 | 34.81 | 35.09 | 35.53 |
| eNAT ™ | 7 | 10⁻³ | 24.69 | 24.48 | 25.12 | 24.94 | 23.73 | 23.53 | 24.10 | 23.84 |
| eNAT ™ | 7 | 10⁻⁴ | 28.11 | 28.18 | 28.35 | 28.32 | 26.74 | 26.82 | 26.94 | 27.04 |
| eNAT ™ | 7 | 10⁻⁵ | 31.37 | 31.45 | 31.76 | 31.59 | 30.19 | 30.28 | 30.25 | 30.31 |
| eNAT ™ | 7 | 10⁻⁶ | 35.93 | 34.85 | 35.59 | 35.02 | 33.35 | 33.65 | 33.65 | 33.57 |
| DB Buffer 1 | 7 | 10⁻³ | 24.08 | 23.94 | 24.98 | 24.69 | 24.08 | 23.87 | 24.65 | 24.28 |
| DB Buffer 1 | 7 | 10⁻⁴ | 27.12 | 27.15 | 27.92 | 27.81 | 27.20 | 27.16 | 27.59 | 27.50 |
| DB Buffer 1 | 7 | 10⁻⁵ | 30.23 | 30.30 | 30.95 | 31.14 | 30.28 | 30.23 | 30.50 | 30.67 |
| DB Buffer 1 | 7 | 10⁻⁶ | 33.12 | 33.67 | 34.37 | 34.95 | 33.66 | 33.90 | 33.88 | 34.17 |
| DB Buffer 2 | 7 | 10⁻³ | 24.06 | 23.94 | 24.91 | 24.69 | 24.02 | 23.80 | 24.51 | 24.30 |
| DB Buffer 2 | 7 | 10⁻⁴ | 26.98 | 27.03 | 27.72 | 27.75 | 26.92 | 26.71 | 27.23 | 27.21 |
| DB Buffer 2 | 7 | 10⁻⁵ | 30.17 | 30.38 | 30.99 | 30.83 | 30.26 | 30.24 | 30.73 | 30.77 |
| DB Buffer 2 | 7 | 10⁻⁶ | 33.59 | 33.13 | 34.30 | 34.37 | 33.50 | 33.93 | 34.23 | 34.46 |
| DB Buffer 3 | 7 | 10⁻³ | 25.02 | 24.98 | 25.47 | 25.40 | 23.51 | 23.37 | 24.02 | 23.64 |
| DB Buffer 3 | 7 | 10⁻⁴ | 28.47 | 28.59 | 29.02 | 28.88 | 26.56 | 26.50 | 26.78 | 26.73 |
| DB Buffer 3 | 7 | 10⁻⁵ | 32.30 | 32.35 | 32.66 | 33.17 | 30.12 | 30.04 | 30.10 | 30.08 |
| DB Buffer 3 | 7 | 10⁻⁶ | 35.52 | 36.00 | 35.69 | 37.02 | 33.39 | 33.65 | 33.30 | 34.18 |
| DB Buffer 4 | 7 | 10⁻³ | 25.11 | 24.87 | 25.70 | 25.33 | 23.58 | 23.21 | 23.90 | 23.52 |
| DB Buffer 4 | 7 | 10⁻⁴ | 28.88 | 28.72 | 29.15 | 29.09 | 26.73 | 26.68 | 26.87 | 26.86 |
| DB Buffer 4 | 7 | 10⁻⁵ | 32.04 | 32.15 | 32.44 | 32.29 | 29.96 | 29.76 | 30.15 | 30.01 |
| DB Buffer 4 | 7 | 10⁻⁶ | 35.45 | 39.88 | 35.47 | 35.72 | 33.43 | 33.06 | 33.34 | 33.31 |
| DB Buffer 5 | 7 | 10⁻³ | 23.81 | 23.72 | 24.30 | 24.19 | 23.82 | 23.58 | 24.05 | 23.69 |
| DB Buffer 5 | 7 | 10⁻⁴ | 27.32 | 27.35 | 27.45 | 27.54 | 26.89 | 26.78 | 27.03 | 26.97 |
| DB Buffer 5 | 7 | 10⁻⁵ | 30.83 | 31.08 | 30.68 | 30.70 | 30.25 | 30.20 | 30.20 | 30.19 |
| DB Buffer 5 | 7 | 10⁻⁶ | 34.65 | 39.41 | 33.89 | 34.18 | 33.43 | 34.06 | 33.95 | 34.05 |
| DB Buffer 6 | 7 | 10⁻³ | 23.65 | 23.57 | 24.21 | 24.11 | 23.80 | 23.42 | 24.02 | 23.55 |
| DB Buffer 6 | 7 | 10⁻⁴ | 27.11 | 27.15 | 27.28 | 27.35 | 26.95 | 26.78 | 26.86 | 26.84 |
| DB Buffer 6 | 7 | 10⁻⁵ | 30.90 | 30.87 | 30.67 | 30.82 | 30.34 | 30.23 | 30.27 | 30.21 |
| DB Buffer 6 | 7 | 10⁻⁶ | 34.93 | 36.77 | 34.21 | 34.01 | 33.86 | 33.92 | 33.54 | 33.80 |

Abbreviations: Dil, Dilution; Rep, Replicate.

TABLE 6
Sample stability data at day 14 after storage at room temperature (RT)
or at 4° C. of DB#1-6 compared to other transport media.

| | Day | | E Gene at | E Gene at | UTR at | UTR at | E Gene at | E gene at | UTR at 4 C. | UTR at 4 C. |
|---|---|---|---|---|---|---|---|---|---|---|
| Buffer | # | Dil | RT Rep 1 | RT Rep 2 | RT Rep 1 | RT Rep 2 | 4 C. Rep 1 | 4 C. Rep 2 | Rep 1 | Rep 2 |
| UTM | 14 | 10⁻³ | 24.26 | 24.15 | 24.85 | 24.56 | 24.39 | 24.22 | 24.90 | 24.84 |
| UTM | 14 | 10⁻⁴ | 28.27 | 28.28 | 28.82 | 28.77 | 27.39 | 27.36 | 27.75 | 27.86 |
| UTM | 14 | 10⁻⁵ | 30.97 | 30.70 | 32.11 | 32.06 | 30.85 | 30.78 | 31.49 | 31.33 |
| UTM | 14 | 10⁻⁶ | 36.08 | 36.21 | 36.71 | 35.91 | 36.67 | 36.85 | 35.65 | 35.88 |
| TE Buffer | 14 | 10⁻³ | 26.70 | 26.18 | 27.04 | 26.36 | 27.91 | 27.56 | 28.48 | 28.24 |
| TE Buffer | 14 | 10⁻⁴ | 31.09 | 31.05 | 30.80 | 30.73 | 32.22 | 31.95 | 32.80 | 32.49 |
| TE Buffer | 14 | 10⁻⁵ | 32.97 | 32.59 | 32.24 | 32.05 | 35.89 | 35.23 | 35.21 | 34.93 |
| TE Buffer | 14 | 10⁻⁶ | 38.86 | 38.99 | 36.73 | 36.65 | 39.13 | 37.62 | 37.64 | 38.26 |
| Lysis Buffer | 14 | 10⁻³ | 24.94 | 24.68 | 24.47 | 24.19 | 24.96 | 24.80 | 25.34 | 25.03 |
| Lysis Buffer | 14 | 10⁻⁴ | 28.65 | 28.57 | 28.07 | 28.11 | 29.04 | 28.79 | 29.18 | 28.78 |
| Lysis Buffer | 14 | 10⁻⁵ | 33.29 | 33.72 | 32.34 | 32.13 | 32.90 | 32.94 | 33.16 | 3.01 |
| Lysis Buffer | 14 | 10⁻⁶ | | | 39.28 | 39.52 | 35.60 | 35.88 | 35.83 | 35.93 |
| eNAT ™ | 14 | 10⁻³ | 25.42 | 25.21 | 24.97 | 24.62 | 26.34 | 26.14 | 26.74 | 26.51 |
| eNAT ™ | 14 | 10⁻⁴ | 29.02 | 29.04 | 28.17 | 28.11 | 28.04 | 28.08 | 28.25 | 28.19 |
| eNAT ™ | 14 | 10⁻⁵ | 32.43 | 32.42 | 31.17 | 31.45 | 32.50 | 32.68 | 33.01 | 33.11 |
| eNAT ™ | 14 | 10⁻⁶ | 36.73 | 36.35 | 35.01 | 34.76 | 34.48 | 34.65 | 35.29 | 35.10 |
| DB Buffer 1 | 14 | 10⁻³ | 24.23 | 24.04 | 24.34 | 24.08 | 24.26 | 24.07 | 24.55 | 24.37 |
| DB Buffer 1 | 14 | 10⁻⁴ | 27.24 | 27.21 | 27.28 | 27.30 | 28.55 | 28.39 | 28.84 | 28.78 |
| DB Buffer 1 | 14 | 10⁻⁵ | 30.36 | 30.11 | 30.36 | 30.33 | 32.09 | 32.05 | 32.36 | 32.24 |
| DB Buffer 1 | 14 | 10⁻⁶ | 34.07 | 33.65 | 34.05 | 34.05 | 37.49 | 34.86 | 35.39 | 36.03 |
| DB Buffer 2 | 14 | 10⁻³ | 24.13 | 23.84 | 24.30 | 24.02 | 24.60 | 24.35 | 25.01 | 24.56 |
| DB Buffer 2 | 14 | 10⁻⁴ | 27.14 | 27.03 | 27.22 | 27.12 | 27.35 | 27.34 | 27.68 | 27.60 |
| DB Buffer 2 | 14 | 10⁻⁵ | 30.46 | 30.46 | 30.55 | 30.61 | 31.39 | 31.22 | 31.78 | 31.98 |
| DB Buffer 2 | 14 | 10⁻⁶ | 34.36 | 34.64 | 34.34 | 34.37 | 34.26 | 35.12 | 34.35 | 35.15 |
| DB Buffer 3 | 14 | 10⁻³ | 26.16 | 25.75 | 25.66 | 25.30 | 24.21 | 24.10 | 24.45 | 24.27 |
| DB Buffer 3 | 14 | 10⁻⁴ | 30.04 | 30.05 | 29.39 | 29.32 | 28.33 | 28.23 | 28.77 | 28.53 |
| DB Buffer 3 | 14 | 10⁻⁵ | 33.75 | 33.97 | 32.67 | 32.84 | 32.60 | 32.13 | 32.51 | 32.32 |
| DB Buffer 3 | 14 | 10⁻⁶ | 36.56 | 37.90 | 37.18 | 36.88 | 37.98 | 35.31 | 36.09 | 36.05 |
| DB Buffer 4 | 14 | 10⁻³ | 26.08 | 25.73 | 25.58 | 25.17 | 24.63 | 24.39 | 24.81 | 24.50 |
| DB Buffer 4 | 14 | 10⁻⁴ | 30.02 | 30.04 | 29.33 | 29.24 | 28.42 | 28.40 | 28.76 | 28.48 |
| DB Buffer 4 | 14 | 10⁻⁵ | 34.37 | 34.42 | 33.13 | 33.19 | 32.86 | 32.50 | 33.13 | 32.89 |
| DB Buffer 4 | 14 | 10⁻⁶ | | 38.81 | 36.96 | 37.84 | 35.67 | 35.28 | 35.50 | 35.73 |
| DB Buffer 5 | 14 | 10⁻³ | 24.05 | 23.81 | 23.74 | 23.50 | 24.45 | 24.28 | 24.63 | 24.36 |
| DB Buffer 5 | 14 | 10⁻⁴ | 27.70 | 27.62 | 27.05 | 26.94 | 28.50 | 28.38 | 28.89 | 28.69 |
| DB Buffer 5 | 14 | 10⁻⁵ | 31.42 | 31.19 | 30.36 | 30.25 | 32.06 | 32.02 | 32.08 | 32.48 |
| DB Buffer 5 | 14 | 10⁻⁶ | 36.10 | 36.62 | 34.23 | 35.03 | 35.00 | 34.38 | 34.17 | 34.38 |
| DB Buffer 6 | 14 | 10⁻³ | 24.09 | 23.75 | 23.96 | 23.43 | 26.12 | 25.95 | 26.30 | 26.08 |
| DB Buffer 6 | 14 | 10⁻⁴ | 27.64 | 27.57 | 27.05 | 27.02 | 28.00 | 28.04 | 28.05 | 28.08 |
| DB Buffer 6 | 14 | 10⁻⁵ | 31.34 | 31.31 | 30.29 | 30.35 | 32.85 | 32.55 | 33.06 | 32.68 |
| DB Buffer 6 | 14 | 10⁻⁶ | 34.51 | 37.32 | 34.05 | 34.23 | 36.14 | 36.14 | 35.85 | 36.30 |

Abbreviations: Dil, Dilution; Rep, Replicate.

TABLE 7
pooling of 4 dilution factors with UTM.

E Gene/UTR/RNase P Triplex PCR
Crossing Threshold (E gene/UTR)

| | No Pool | Pool 4 with UTM | Pool 4 with NPS |
|---|---|---|---|
| 20306 | 16.03/18.33 | 15.39/18.02 | 17.34/19.46 |
| 18007 | 32.73/34.12 | 35.38/37.57 | 0/40.53 |
| 18016 | 32.58/34.04 | 35.25/36.77 | 0/39.01 |
| 17913 | 27.58/29.05 | 30.22/32.73 | 31.67/33.75 |
| 20245 | 33.06/34.48 | 33.42/34.81 | 35.47/39.14 |
| 18025 | 34.12/35.47 | 37.39/42.96 | 0/0 |
| 20326 | 27.86/29.27 | 29.49/31.50 | 31.05/32.63 |
| 20330 | 31.87/33.33 | 35.16/36.86 | 0/41.80 |
| 17925 | 20.92/22.49 | 22.13/24.22 | 24.14/25.89 |

TABLE 8
pooling results with COVID-positive samples collected in COBAS.

| | E Gene/UTR/RNase P Triplex PCR | | | |
|---|---|---|---|---|
| | Crossing Threshold (E gene/UTR) | | | |
| | | | Condition C | |
| | | | 500 μl to 80 μl | |
| | Condition A | | 125 μl | |
| | No Pool | Condition D | Sample + | 125 μl |
| | 250 μl to | 500 μl to | 375 μl Lysis | Sample + |
| | 80 μl | 80 μl | Buffer | 375 μl UTM |
| 19826 | 32.50/34.49 | 31.60/33.62 | 32.21/34.34 | 34.82/37.30 |
| 20315 | 31.88/33.80 | 31.16/33.17 | 31.56/33.45 | 34.28/36.46 |
| 19575 | 21.05/22.89 | 20.67/22.53 | 20.13/21.73 | 22.88/24.87 |
| 19778 | 35.17/35.36 | 34.12/35.99 | 33.66/35.47 | 36.86/39.12 |

TABLE 9
confirmed results of COVID-19 collected in COBAS.

| | E Gene/UTR/RNase P Triplex PCR | | |
|---|---|---|---|
| | Crossing Threshold (E gene/UTR) | | |
| | | Condition C | |
| | Condition A | 500 μl to 80 μl | |
| | No Pool | 125 μl Sample + | 125 μl Sample + |
| | 250 μl to 70 μl | 375 μl Lysis Buffer | 375 μl UTM |
| 19991 | 26.40/28.12 | 25.63/27.18 | 26.23/28.25 |
| 19988 | 19.79/21.38 | 19.16/20.55 | 20.28/22.37 |
| 19559 | 28.17/30.02 | 27.21/28.93 | 28.85/31.20 |
| 19847 | 21.77/24.09 | 20.69/22.93 | 22.67/25.00 |
| 19679 | 26.87/28.04 | 26.47/27.56 | 27.95/29.37 |
| 19770 | 19.45/21.06 | 18.83/20.22 | 20.35/22.30 |
| 19521 | 34.82/36.54 | 34.81/35.57 | 40.06/0 |
| 19523 | 31.18/34.32 | 30.69/33.59 | 32.84/36.31 |

TABLE 10
results from the pooling of real samples.

| | E Gene/UTR/RNase P Triplex PCR | | | |
|---|---|---|---|---|
| | Crossing Threshold (E gene/UTR) | | | |
| | | Condition C | | |
| | | 500 μl to 80 μl | | |
| | Condition A | 125 μl | | 125 μl |
| | No Pool | Sample + | 125 μl | Sample + |
| | 250 μl to | 375 μl Lysis | Sample + | 375 μl of 3 NPS |
| | 70 μl | Buffer | 375 μl UTM | in Lysis Buffer |
| 19620 | 29.23/31.14 | 27.95/28.49 | 27.95/28.61 | 28.58/30.33 |
| 19691 | 28.98/31.27 | 28.03/29.96 | 28.65/30.99 | 29.01/31.32 |
| 19662 | 22.87/24.68 | 21.21/22.71 | 22.52/24.22 | 24.01/25.55 |
| 19692 | 35.55/37.78 | 35.33/37.30 | 42.21/42.36 | 37.53/39.47 |
| 19779 | 27.76/28.98 | 27.40/28.03 | 28.05/29.39 | 29.25/30.08 |
| 19886 | 22.92/24.06 | 22.68/24.24 | 23.05/24.62 | 24.06/25.55 |

TABLE 11
pooling compatibility with DB#1-6.

| | COVID Triplex PCR |
|---|---|
| Sample ID | Crossing Threshold (E Gene/UTR) |
| 19770 No Pool | 20.88/22.97 |
| 19770, DB Buffer 1 | 21.52/23.64 |
| 19770, DB Buffer 2 | 21.85/24.03 |
| 19770, DB Buffer 3 | 19.67/21.18 |
| 19770, DB Buffer 4 | 19.70/21.16 |
| 19770, DB Buffer 5 | 19.20/20.77 |
| 19770, DB Buffer 6 | 19.49/21.07 |
| 19523, No Pool | 31.84/34.43 |
| 19523, DB Buffer 1 | 34.07/36.82 |
| 19523, DB Buffer 2 | 34.04/38.39 |
| 19523, DB Buffer 3 | 31.57/34.40 |
| 19523, DB Buffer 4 | NA |
| 19523, DB Buffer 5 | 31.12/34.40 |
| 19523, DB Buffer 6 | NA |
| 19559 No Pool | 28.99/30.84 |
| 19559, DB Buffer 1 | 30.82/32.97 |
| 19559, DB Buffer 2 | 30.93/33.07 |
| 19559, DB Buffer 3 | 28.19/29.80 |
| 19559, DB Buffer 4 | 28.44/30.01 |
| 19559, DB Buffer 5 | 27.85/29.38 |
| 19559, DB Buffer 6 | 27.77/29.43 |

TABLE 12
crossing thresholds from pooling 4 or 8
samples in DB#5 compared to non-pooled.

| | COVID Triplex PCR | | | |
|---|---|---|---|---|
| | Crossing Thresholds (E Gene/UTR) | | | |
| | No Pool in | No Pool in DB | Pool 4 in DB | Pool 8 in DB |
| | Lysis Buffer | Buffer 5 | Buffer 5 | Buffer 5 |
| 24193 10⁻³ | 26.47/27.30 | 25.55/26.22 | 26.76/26.76 | 25.26/25.77 |
| | 26.11/26.77 | 25.37/25.89 | 26.57/26.57 | 25.19/25.46 |
| 24193 10⁻⁴ | 28.53/29.17 | 27.07/27.41 | 27.48/27.42 | 27.01/27.09 |
| | 28.06/28.37 | 26.57/26.92 | 27.29/27.19 | 26.80/26.83 |
| 24193 10⁻⁵ | 31.84/32.63 | 30.27/30.39 | 31.22/31.08 | 30.36/30.73 |
| | 31.54/31.91 | 30.11/30.14 | 31.11/30.60 | 30.26/30.42 |
| 24193 10⁻⁶ | 36.26/35.18 | 34.03/33.85 | 33.76/33.35 | 32.95/33.08 |
| | 34.48/34.39 | 32.99/33.08 | 33.65/33.11 | 33.11/32.65 |
| 24570 10⁻³ | 29.18/31.25 | 28.21/30.19 | 28.58/30.41 | 27.35/29.20 |
| | 28.52/30.59 | 27.86/29.78 | 28.22/30.04 | 27.14/28.99 |
| 24570 10⁻⁴ | 32.50/34.18 | 31.46/33.09 | 31.69/33.48 | 31.02/32.74 |
| | 31.85/33.25 | 31.01/32.75 | 31.44/32.79 | 30.93/32.27 |
| 24570 10⁻⁵ | 38.48/39.67 | 35.68/37.20 | 37.56/38.10 | 35.50/37.47 |
| | 36.29/0 | 36.32/37.25 | 36.49/39.28 | 35.09/35.93 |
| 24570 10⁻⁶ | 38.72/39.02 | 0/38.20 | 0/39.73 | 36.30/37.25 |
| | 36.21/0 | 38.40/37.87 | 0/38.21 | 36.53/37.92 |

TABLE 13
crossing thresholds from mocked clinical SARS-
CoV-2 samples spiked into DB#5 and stored
up to 60 days at 4 C., room temperature, and 37 C.

| | | COVID Triplex | | | |
|---|---|---|---|---|---|
| Replicate # | Day | E Gene | UTR | RNase P | MS2 |

RT Storage

| | | | | | |
|---|---|---|---|---|---|
| 1 | 0 | 27.22 | 28.20 | 27.66 | 26.40 |
| 2 | 0 | 27.06 | 27.84 | 27.46 | 26.13 |
| 3 | 0 | 26.94 | 27.99 | 27.36 | 26.15 |
| 4 | 0 | 26.81 | 27.68 | 27.23 | 25.99 |
| 5 | 0 | 26.79 | 27.69 | 27.26 | 26.13 |
| 6 | 0 | 26.85 | 27.73 | 27.39 | 26.26 |
| 7 | 0 | 26.77 | 27.69 | 27.12 | 25.84 |
| 8 | 0 | 26.66 | 27.44 | 26.71 | 26.07 |
| 9 | 0 | 26.95 | 27.89 | 27.31 | 25.83 |
| 10 | 0 | 26.79 | 27.63 | 27.31 | 25.55 |
| 11 | 0 | 26.73 | 27.55 | 27.16 | 25.48 |
| 12 | 0 | 26.71 | 27.49 | 27.23 | 25.72 |
| 13 | 0 | 26.78 | 27.54 | 27.11 | 26.05 |
| 14 | 0 | 27.09 | 27.79 | 27.67 | 26.56 |
| 15 | 0 | 26.85 | 27.66 | 27.19 | 26.10 |
| 16 | 0 | 28.51 | 29.68 | 27.86 | 27.51 |
| 17 | 0 | 27.02 | 27.89 | 27.51 | 25.11 |
| 18 | 0 | 26.86 | 27.60 | 27.47 | 26.25 |
| 19 | 0 | 26.64 | 27.30 | 27.12 | 25.63 |
| 20 | 0 | 26.51 | 27.33 | 27.09 | 25.81 |
| Mean | 0 | 26.93 | 27.78 | 27.31 | 26.03 |
| 1 | 7 | 26.55 | 27.85 | 27.03 | 26.20 |
| 2 | 7 | 26.65 | 28.05 | 27.20 | 26.23 |
| 3 | 7 | 26.41 | 27.60 | 27.17 | 26.10 |
| 4 | 7 | 26.28 | 27.52 | 26.88 | 26.01 |
| 5 | 7 | 26.73 | 28.31 | 27.27 | 26.17 |
| 6 | 7 | 26.68 | 28.15 | 27.26 | 25.90 |
| 7 | 7 | 26.44 | 27.49 | 26.89 | 25.84 |
| 8 | 7 | 26.27 | 27.46 | 26.69 | 26.26 |
| 9 | 7 | 26.41 | 27.60 | 27.01 | 26.00 |
| 10 | 7 | 26.21 | 27.48 | 26.95 | 26.14 |
| 11 | 7 | 26.30 | 27.47 | 27.00 | 26.03 |
| 12 | 7 | 26.22 | 27.42 | 26.91 | 26.05 |
| 13 | 7 | 26.33 | 27.49 | 26.94 | 26.08 |
| 14 | 7 | 26.25 | 27.37 | 26.97 | 26.18 |
| 15 | 7 | 26.49 | 27.67 | 27.07 | Excluded |
| 16 | 7 | 26.40 | 27.62 | 27.02 | 27.97 |
| 17 | 7 | 26.43 | 27.67 | 27.02 | 25.84 |
| 18 | 7 | 26.31 | 27.45 | 27.03 | 26.23 |
| 19 | 7 | 26.31 | 27.48 | 27.04 | 25.91 |
| 20 | 7 | 26.32 | 27.41 | 26.84 | Excluded |
| Mean | | 26.40 | 27.63 | 27.01 | 26.17 |
| 1 | 14 | 26.68 | 27.84 | 27.34 | 26.22 |
| 2 | 14 | 26.56 | 27.75 | 27.29 | 26.30 |
| 3 | 14 | 26.55 | 27.59 | 27.20 | 25.73 |
| 4 | 14 | 26.51 | 27.64 | 27.29 | 25.93 |
| 5 | 14 | 26.64 | 27.81 | 27.20 | 25.60 |
| 6 | 14 | 26.59 | 27.75 | 27.16 | 25.98 |
| 7 | 14 | 26.63 | 27.72 | 27.14 | 25.82 |
| 8 | 14 | 26.56 | 27.88 | 27.15 | 26.46 |
| 9 | 14 | 26.60 | 27.72 | 27.41 | 25.93 |
| 10 | 14 | 26.42 | 27.54 | 27.11 | 26.20 |
| 11 | 14 | 26.51 | 27.51 | 27.17 | 25.80 |
| 12 | 14 | 26.51 | 27.67 | 27.23 | 26.02 |
| 13 | 14 | 26.62 | 27.68 | 27.15 | 25.97 |
| 14 | 14 | 26.51 | 27.57 | 27.21 | 26.51 |
| 15 | 14 | 26.72 | 27.86 | 27.33 | 26.05 |
| 16 | 14 | Excluded | Excluded | Excluded | 26.25 |
| 17 | 14 | 26.58 | 27.71 | 27.18 | 26.18 |
| 18 | 14 | 26.48 | 27.59 | 27.39 | 26.34 |
| 19 | 14 | 26.42 | 27.42 | 27.11 | 26.03 |
| 20 | 14 | 26.51 | 27.67 | 27.14 | 26.14 |
| Mean | | 26.56 | 27.68 | 27.22 | 26.07 |
| 1 | 30 | 27.19 | 28.12 | 28.42 | 26.17 |
| 2 | 30 | 26.90 | 27.97 | 28.02 | 26.06 |
| 3 | 30 | 27.26 | 28.26 | 28.33 | Excluded |
| 4 | 30 | 26.95 | 28.02 | 27.84 | 25.97 |
| 5 | 30 | 26.97 | 27.82 | 27.71 | 25.85 |
| 6 | 30 | 27.07 | 28.04 | 27.95 | 25.74 |
| 7 | 30 | 27.05 | 28.07 | 27.94 | 26.04 |
| 8 | 30 | 27.09 | 28.10 | 27.86 | 25.17 |
| 9 | 30 | 27.09 | 28.07 | 27.98 | 26.01 |
| 10 | 30 | 27.00 | 27.93 | 28.02 | 25.57 |
| 11 | 30 | 26.87 | 27.84 | 27.78 | 25.64 |
| 12 | 30 | 26.88 | 27.81 | 27.77 | 25.99 |
| 13 | 30 | 26.90 | 28.01 | 27.78 | 25.58 |
| 14 | 30 | 26.82 | 27.93 | 27.85 | 25.98 |
| 15 | 30 | 26.93 | 28.04 | 27.87 | 26.04 |
| 16 | 30 | 27.09 | 28.12 | 27.80 | Excluded |
| 17 | 30 | 27.19 | 28.16 | 28.32 | 26.30 |
| 18 | 30 | 27.03 | 28.03 | 28.31 | 26.19 |
| 19 | 30 | 26.96 | 28.05 | 28.07 | 26.23 |
| 20 | 30 | 26.99 | 27.91 | 28.02 | 26.27 |
| Mean | | 27.01 | 28.01 | 27.98 | 25.93 |
| 1 | 60 | 27.92 | 28.48 | 28.18 | 26.16 |
| 2 | 60 | 27.72 | 28.34 | 27.92 | 25.96 |
| 3 | 60 | 27.35 | 28.06 | 27.25 | 26.17 |
| 4 | 60 | 27.79 | 28.46 | 28.11 | 25.77 |
| 5 | 60 | 27.85 | 28.47 | 28.13 | 25.69 |
| 6 | 60 | 27.39 | 27.96 | 27.23 | 25.26 |
| 7 | 60 | 27.43 | 27.98 | 27.23 | 25.83 |
| 8 | 60 | 27.36 | 27.77 | 27.06 | 25.50 |
| 9 | 60 | 28.58 | 29.06 | 28.56 | 26.01 |
| 10 | 60 | 27.80 | 28.26 | 28.01 | 25.70 |
| 11 | 60 | 27.75 | 28.29 | 28.08 | 25.56 |
| 12 | 60 | 27.78 | 28.38 | 28.07 | 25.35 |
| 13 | 60 | 27.69 | 28.23 | 28.01 | 25.57 |
| 14 | 60 | 27.77 | 28.29 | 27.97 | 25.43 |
| 15 | 60 | 27.89 | 28.35 | 28.02 | 25.79 |
| 16 | 60 | 27.86 | 28.43 | 28.10 | 25.48 |
| 17 | 60 | 27.83 | 28.49 | 28.05 | 25.82 |
| 18 | 60 | 27.87 | 28.37 | 28.17 | 25.73 |
| 19 | 60 | 27.57 | 28.16 | 28.03 | 25.14 |
| 20 | 60 | 27.55 | 28.17 | 27.81 | 25.43 |
| Mean | 60 | 27.74 | 28.30 | 27.90 | 25.67 |

4 C. Storage

| | | | | | |
|---|---|---|---|---|---|
| 1 | 7 | 26.63 | 27.64 | 27.02 | 25.80 |
| 2 | 7 | 26.78 | 27.70 | 27.00 | 25.87 |
| 3 | 7 | 26.27 | 27.18 | 27.95 | 25.84 |
| 4 | 7 | 26.20 | 27.18 | 27.47 | 26.08 |
| 5 | 7 | 26.62 | 27.62 | 26.86 | 25.89 |
| 6 | 7 | 26.48 | 27.43 | 26.89 | 25.90 |
| 7 | 7 | 26.57 | 27.50 | 26.80 | 25.68 |
| 8 | 7 | 26.66 | 27.65 | 26.99 | 25.69 |
| 9 | 7 | 26.60 | 27.60 | 26.80 | 25.60 |
| 10 | 7 | 26.15 | 26.56 | 26.31 | 25.76 |
| 11 | 7 | 26.48 | 27.44 | 26.77 | 25.97 |
| 12 | 7 | 26.45 | 27.50 | 26.85 | 27.77 |
| 13 | 7 | 26.48 | 27.54 | 26.86 | 25.93 |
| 14 | 7 | 26.44 | 27.51 | 26.80 | 25.99 |
| 15 | 7 | 26.68 | 27.61 | 27.03 | 25.61 |
| 16 | 7 | 26.46 | 27.54 | 26.83 | 25.68 |
| 17 | 7 | 26.53 | 27.23 | 26.65 | 25.88 |
| 18 | 7 | 2645.00 | 27.54 | 26.83 | 25.83 |
| 19 | 7 | Excluded | Excluded | Excluded | 25.91 |
| 20 | 7 | Excluded | Excluded | Excluded | 26.17 |
| Mean | | 26.50 | 27.44 | 26.93 | 25.94 |
| 1 | 14 | 26.68 | 27.68 | 27.05 | 25.61 |
| 2 | 14 | 26.70 | 27.77 | 27.21 | 26.05 |
| 3 | 14 | 26.73 | 27.80 | 27.12 | 26.06 |
| 4 | 14 | 26.71 | 27.86 | 27.14 | 27.08 |
| 5 | 14 | 26.78 | 27.76 | 27.20 | 25.69 |
| 6 | 14 | 26.58 | 27.62 | 27.15 | 25.94 |
| 7 | 14 | 26.52 | 27.48 | 27.02 | 25.44 |
| 8 | 14 | 26.46 | 27.51 | 27.05 | 26.03 |
| 9 | 14 | 26.59 | 27.58 | 27.10 | 25.47 |
| 10 | 14 | 26.54 | 27.63 | 26.97 | 26.02 |
| 11 | 14 | 26.71 | 27.72 | 27.06 | 25.80 |
| 12 | 14 | 26.99 | 28.34 | 27.37 | 26.11 |
| 13 | 14 | 26.69 | 27.75 | 27.13 | 25.82 |
| 14 | 14 | 26.62 | 27.62 | 27.09 | 25.72 |
| 15 | 14 | 26.69 | 27.52 | 27.03 | 25.59 |
| 16 | 14 | 26.55 | 27.64 | 26.97 | 25.89 |
| 17 | 14 | 26.56 | 27.68 | 27.14 | 25.72 |
| 18 | 14 | 26.63 | 27.66 | 27.20 | 25.85 |
| 19 | 14 | 26.75 | 27.84 | 27.22 | 26.04 |
| 20 | 14 | 27.10 | 28.36 | 27.53 | Excluded |
| Mean | | 26.68 | 27.74 | 27.14 | 25.89 |
| 1 | 30 | 26.80 | 27.67 | 28.16 | 25.55 |
| 2 | 30 | 26.93 | 27.54 | 27.86 | 25.87 |
| 3 | 30 | 27.07 | 28.09 | 28.36 | 25.98 |
| 4 | 30 | 26.97 | 27.87 | 28.15 | Excluded |
| 5 | 30 | 26.96 | 27.85 | 28.17 | 25.84 |
| 6 | 30 | 26.67 | 27.63 | 27.82 | 25.57 |
| 7 | 30 | 26.86 | 27.71 | 28.04 | 25.53 |
| 8 | 30 | 26.71 | 27.51 | 27.73 | 25.44 |
| 9 | 30 | 26.70 | 27.54 | 27.66 | 25.47 |
| 10 | 30 | 26.83 | 27.68 | 27.91 | 25.58 |
| 11 | 30 | 26.67 | 27.60 | 27.87 | 25.55 |
| 12 | 30 | 27.08 | 27.79 | 27.85 | 25.67 |
| 13 | 30 | 26.77 | 27.85 | 27.95 | 25.84 |
| 14 | 30 | 26.61 | 27.50 | 28.03 | 26.08 |
| 15 | 30 | 26.65 | 27.51 | 27.93 | 25.59 |
| 16 | 30 | 26.50 | 27.46 | 27.71 | 25.56 |
| 17 | 30 | 26.71 | 27.52 | 27.77 | 25.66 |
| 18 | 30 | 26.67 | 27.68 | 27.73 | 25.65 |
| 19 | 30 | 26.69 | 27.64 | 27.78 | 25.56 |
| 20 | 30 | 26.96 | 28.03 | 28.09 | 26.77 |
| Mean | | 26.79 | 27.68 | 27.93 | 25.72 |
| 1 | 60 | 26.48 | 27.30 | 27.79 | 24.40 |
| 2 | 60 | 26.51 | 27.28 | 27.80 | 24.34 |
| 3 | 60 | 26.61 | 27.53 | 27.89 | 24.40 |
| 4 | 60 | 26.60 | 27.53 | 27.99 | Excluded |
| 5 | 60 | 26.71 | 27.69 | 28.14 | 24.52 |
| 6 | 60 | 26.62 | 27.54 | 27.98 | 24.65 |
| 7 | 60 | 26.51 | 27.39 | 27.87 | 24.39 |
| 8 | 60 | 26.46 | 27.33 | 27.79 | 24.28 |
| 9 | 60 | 26.60 | 27.54 | 27.89 | 24.83 |
| 10 | 60 | 26.42 | 27.29 | 27.76 | 24.95 |
| 11 | 60 | 26.66 | 27.67 | 28.13 | 24.93 |
| 12 | 60 | 26.62 | 27.55 | 27.76 | 24.90 |
| 13 | 60 | 26.68 | 27.61 | 28.00 | 25.44 |
| 14 | 60 | 26.63 | 27.58 | 27.82 | 25.05 |
| 15 | 60 | 26.53 | 27.39 | 27.81 | 24.79 |
| 16 | 60 | 26.51 | 27.49 | 27.76 | 24.79 |
| 17 | 60 | 26.46 | 27.44 | 27.95 | 24.74 |
| 18 | 60 | 26.53 | 27.41 | 28.01 | 24.64 |
| 19 | 60 | 26.62 | 27.55 | 27.76 | 24.63 |
| 20 | 60 | 26.78 | 27.72 | 28.06 | 24.97 |
| Mean | 60 | 26.58 | 27.49 | 27.90 | 24.72 |

37 C. Storage

| | | | | | |
|---|---|---|---|---|---|
| 1 | 7 | 27.57 | 28.38 | 27.06 | 26.96 |
| 2 | 7 | 27.36 | 28.14 | 27.03 | 26.77 |
| 3 | 7 | 27.42 | 28.14 | 26.91 | 26.69 |
| 4 | 7 | 27.33 | 28.11 | 26.83 | 27.15 |
| 5 | 7 | 27.32 | 28.18 | 26.74 | 26.97 |
| 6 | 7 | 27.59 | 28.49 | 27.10 | 26.66 |
| 7 | 7 | 27.38 | 28.11 | 27.03 | 27.05 |
| 8 | 7 | 27.48 | 28.20 | 26.99 | 27.00 |
| 9 | 7 | 27.39 | 28.05 | 26.71 | 26.64 |
| 10 | 7 | 27.43 | 28.11 | 26.72 | 26.63 |
| 11 | 7 | 27.33 | 28.01 | 26.84 | 26.61 |
| 12 | 7 | 27.46 | 28.20 | 27.04 | 26.76 |
| 13 | 7 | 27.34 | 28.01 | 26.77 | 26.68 |
| 14 | 7 | 27.45 | 28.14 | 26.79 | 26.81 |
| 15 | 7 | 27.62 | 28.22 | 26.83 | 27.06 |
| 16 | 7 | 27.39 | 28.16 | 26.83 | 27.18 |
| 17 | 7 | 27.74 | 28.27 | 27.08 | 27.50 |
| 18 | 7 | 27.23 | 27.97 | 26.74 | 26.99 |
| 19 | 7 | 27.45 | 28.06 | 26.71 | 27.29 |
| 20 | 7 | 27.34 | 28.16 | 26.72 | 27.03 |
| Mean | | 27.43 | 28.16 | 26.87 | 26.92 |
| 1 | 14 | 29.68 | 29.52 | 27.51 | 28.35 |
| 2 | 14 | 29.56 | 29.33 | 27.34 | 28.21 |
| 3 | 14 | 29.60 | 29.50 | 27.42 | 27.79 |
| 4 | 14 | 29.49 | 29.40 | 27.17 | 28.11 |
| 5 | 14 | 29.46 | 29.42 | 27.44 | 27.93 |
| 6 | 14 | 29.41 | 29.51 | 27.37 | 28.18 |
| 7 | 14 | 29.40 | 29.46 | 27.24 | 27.96 |
| 8 | 14 | 29.02 | 28.86 | 26.29 | 28.25 |
| 9 | 14 | 29.63 | 29.18 | 27.52 | 27.88 |
| 10 | 14 | 29.26 | 29.13 | 27.03 | 27.66 |
| 11 | 14 | 29.27 | 29.14 | 27.09 | 27.74 |
| 12 | 14 | 29.22 | 29.09 | 27.02 | 27.59 |
| 13 | 14 | 29.33 | 29.21 | 27.16 | 27.69 |
| 14 | 14 | 29.28 | 29.22 | 27.11 | 27.96 |
| 15 | 14 | 29.30 | 29.20 | 27.10 | 27.63 |
| 16 | 14 | 29.41 | 29.32 | 27.15 | 28.05 |
| 17 | 14 | 29.17 | 28.75 | 26.86 | 27.60 |
| 18 | 14 | 28.79 | 28.04 | 25.64 | 27.45 |
| 19 | 14 | 29.29 | 29.29 | 26.41 | 27.53 |
| 20 | 14 | 29.37 | 29.25 | 27.05 | 28.00 |
| Mean | | 29.35 | 29.19 | 27.05 | 27.88 |
| 1 | 30 | 33.31 | 31.31 | 28.47 | Excluded |
| 2 | 30 | 32.76 | 31.12 | 27.92 | 28.86 |
| 3 | 30 | 33.12 | 31.21 | 27.89 | 29.02 |
| 4 | 30 | 33.05 | 30.50 | 27.68 | 28.69 |
| 5 | 30 | 33.52 | 31.52 | 27.93 | 29.02 |
| 6 | 30 | 33.32 | 31.32 | 27.97 | 29.02 |
| 7 | 30 | 33.36 | 31.65 | 28.13 | 29.32 |
| 8 | 30 | 33.94 | 31.44 | 27.96 | 29.29 |
| 9 | 30 | 34.05 | 32.00 | 28.22 | 29.64 |
| 10 | 30 | 33.21 | 31.87 | 28.29 | 29.26 |
| 11 | 30 | 33.59 | 31.72 | 28.25 | 29.17 |
| 12 | 30 | 33.59 | 31.51 | 27.92 | 29.10 |
| 13 | 30 | 33.27 | 31.62 | 28.26 | 29.08 |
| 14 | 30 | 33.48 | 31.54 | 27.94 | 29.12 |
| 15 | 30 | 33.57 | 31.73 | 28.16 | 29.35 |
| 16 | 30 | 34.32 | 31.60 | 27.95 | 29.23 |
| 17 | 30 | 33.06 | 31.17 | 27.03 | 28.95 |
| 18 | 30 | 32.62 | 31.26 | 28.06 | 29.24 |
| 19 | 30 | 33.08 | 31.38 | 28.05 | 29.20 |
| 20 | 30 | 34.48 | 31.56 | 28.11 | 29.20 |
| Mean | | 33.44 | 31.45 | 28.01 | 29.15 |
| 1 | 60 | 37.30 | 34.24 | 28.04 | 30.44 |
| 2 | 60 | 37.46 | 34.18 | 28.05 | 30.28 |
| 3 | 60 | 37.18 | 34.09 | 28.21 | 30.53 |
| 4 | 60 | 0.00 | 34.08 | 28.07 | 30.29 |
| 5 | 60 | 42.45 | 34.07 | 28.13 | 30.26 |
| 6 | 60 | 35.72 | 34.05 | 27.91 | 30.39 |
| 7 | 60 | 36.62 | 34.08 | 28.24 | 30.38 |
| 8 | 60 | 37.77 | 34.53 | 28.19 | Excluded |
| 9 | 60 | 38.89 | 34.63 | 28.12 | Excluded |
| 10 | 60 | 38.60 | 34.07 | 28.08 | 30.09 |
| 11 | 60 | 36.16 | 35.01 | 27.84 | 30.17 |
| 12 | 60 | 35.98 | 33.80 | 27.90 | 30.19 |
| 13 | 60 | 37.46 | 34.21 | 27.86 | 30.01 |
| 14 | 60 | 36.53 | 33.53 | 27.86 | 30.06 |
| 15 | 60 | 38.26 | 34.16 | 28.01 | 30.26 |
| 16 | 60 | 35.99 | 34.29 | 27.94 | 30.37 |
| 17 | 60 | 35.29 | 34.50 | 27.80 | 30.73 |
| 18 | 60 | 37.91 | 34.43 | 28.02 | 30.23 |
| 19 | 60 | 39.82 | 34.23 | 28.11 | 30.20 |
| 20 | 60 | 36.74 | 33.93 | 27.88 | 30.17 |
| Mean | 60 | 37.48 | 34.21 | 28.01 | 30.28 |

TABLE 14
crossing thresholds from mocked clinical SARS-CoV-2 samples spiked into DB#5 and stored up to
270 days at 4 C., room temperature, and 37 C. Some replicates were excluded due to low quality data.

| | | RT Storage | | | | 4 C. Storage | | | | 37 C. Storage | | | |
|---|---|---|---|---|---|---|---|---|---|---|---|---|---|
| | | COVID | | | | COVID | | | | COVID | | | |
| Rep. | | Triplex | | | | Triplex | | | | Triplex | | | |
| # | Day | E Gene | UTR | RNase P | MS2 | E Gene | UTR | RNase | MS2 | E Gene | UTR | RNase P | MS2 |
| 1 | 0 | 27.22 | 28.20 | 27.66 | 26.40 | NA | NA | NA | NA | NA | NA | NA | NA |
| 2 | 0 | 27.06 | 27.84 | 27.46 | 26.13 | NA | NA | NA | NA | NA | NA | NA | NA |
| 3 | 0 | 26.94 | 27.99 | 27.36 | 26.15 | NA | NA | NA | NA | NA | NA | NA | NA |
| 4 | 0 | 26.81 | 27.68 | 27.23 | 25.99 | NA | NA | NA | NA | NA | NA | NA | NA |
| 5 | 0 | 26.79 | 27.69 | 27.26 | 26.13 | NA | NA | NA | NA | NA | NA | NA | NA |
| 6 | 0 | 26.85 | 27.73 | 27.39 | 26.26 | NA | NA | NA | NA | NA | NA | NA | NA |
| 7 | 0 | 26.77 | 27.69 | 27.12 | 25.84 | NA | NA | NA | NA | NA | NA | NA | NA |
| 8 | 0 | 26.66 | 27.44 | 26.71 | 26.07 | NA | NA | NA | NA | NA | NA | NA | NA |
| 9 | 0 | 26.95 | 27.89 | 27.31 | 25.83 | NA | NA | NA | NA | NA | NA | NA | NA |
| 10 | 0 | 26.79 | 27.63 | 27.31 | 25.55 | NA | NA | NA | NA | NA | NA | NA | NA |
| 11 | 0 | 26.73 | 27.55 | 27.16 | 25.48 | NA | NA | NA | NA | NA | NA | NA | NA |
| 12 | 0 | 26.71 | 27.49 | 27.23 | 25.72 | NA | NA | NA | NA | NA | NA | NA | NA |
| 13 | 0 | 26.78 | 27.54 | 27.11 | 26.05 | NA | NA | NA | NA | NA | NA | NA | NA |
| 14 | 0 | 27.09 | 27.79 | 27.67 | 26.56 | NA | NA | NA | NA | NA | NA | NA | NA |
| 15 | 0 | 26.85 | 27.66 | 27.19 | 26.10 | NA | NA | NA | NA | NA | NA | NA | NA |
| 16 | 0 | 28.51 | 29.68 | 27.86 | 27.51 | NA | NA | NA | NA | NA | NA | NA | NA |
| 17 | 0 | 27.02 | 27.89 | 27.51 | 25.11 | NA | NA | NA | NA | NA | NA | NA | NA |
| 18 | 0 | 26.86 | 27.60 | 27.47 | 26.25 | NA | NA | NA | NA | NA | NA | NA | NA |
| 19 | 0 | 26.64 | 27.30 | 27.12 | 25.63 | NA | NA | NA | NA | NA | NA | NA | NA |
| 20 | 0 | 26.51 | 27.33 | 27.09 | 25.81 | NA | NA | NA | NA | NA | NA | NA | NA |
| Mean | | 26.93 | 27.78 | 27.31 | 29.53 | NA | NA | NA | NA | NA | NA | NA | NA |
| 1 | 7 | 26.55 | 27.85 | 27.03 | 26.20 | 26.63 | 27.64 | 27.02 | 25.80 | 27.57 | 28.38 | 27.06 | 26.96 |
| 2 | 7 | 26.65 | 28.05 | 27.20 | 26.23 | 26.78 | 27.70 | 27.00 | 25.87 | 27.36 | 28.14 | 27.03 | 26.77 |
| 3 | 7 | 26.41 | 27.60 | 27.17 | 26.10 | 26.27 | 27.18 | 27.95 | 25.84 | 27.42 | 28.14 | 26.91 | 26.69 |
| 4 | 7 | 26.28 | 27.52 | 26.88 | 26.01 | 26.20 | 27.18 | 27.47 | 26.08 | 27.33 | 28.11 | 26.83 | 27.15 |
| 5 | 7 | 26.73 | 28.31 | 27.27 | 26.17 | 26.62 | 27.62 | 26.86 | 25.89 | 27.32 | 28.18 | 26.74 | 26.97 |
| 6 | 7 | 26.68 | 28.15 | 27.26 | 25.90 | 26.48 | 27.43 | 26.89 | 25.90 | 27.59 | 28.49 | 27.10 | 26.66 |
| 7 | 7 | 26.44 | 27.49 | 26.89 | 25.84 | 26.57 | 27.50 | 26.80 | 25.68 | 27.38 | 28.11 | 27.03 | 27.05 |
| 8 | 7 | 26.27 | 27.46 | 26.69 | 26.26 | 26.66 | 27.65 | 26.99 | 25.69 | 27.48 | 28.20 | 26.99 | 27.00 |
| 9 | 7 | 26.41 | 27.60 | 27.01 | 26.00 | 26.60 | 27.60 | 26.80 | 25.60 | 27.39 | 28.05 | 26.71 | 26.64 |
| 10 | 7 | 26.21 | 27.48 | 26.95 | 26.14 | 26.15 | 26.56 | 26.31 | 25.76 | 27.43 | 28.11 | 26.72 | 26.63 |
| 11 | 7 | 26.30 | 27.47 | 27.00 | 26.03 | 26.48 | 27.44 | 26.77 | 25.97 | 27.33 | 28.01 | 26.84 | 26.61 |
| 12 | 7 | 26.22 | 27.42 | 26.91 | 26.05 | 26.45 | 27.50 | 26.85 | 27.77 | 27.46 | 28.20 | 27.04 | 26.76 |
| 13 | 7 | 26.33 | 27.49 | 26.94 | 26.08 | 26.48 | 27.54 | 26.86 | 25.93 | 27.34 | 28.01 | 26.77 | 26.68 |
| 14 | 7 | 26.25 | 27.37 | 26.97 | 26.18 | 26.44 | 27.51 | 26.80 | 25.99 | 27.45 | 28.14 | 26.79 | 26.81 |
| 15 | 7 | 26.49 | 27.67 | 27.07 | Excluded | 26.68 | 27.61 | 27.03 | 25.61 | 27.62 | 28.22 | 26.83 | 27.06 |
| 16 | 7 | 26.40 | 27.62 | 27.02 | 27.97 | 26.46 | 27.54 | 26.83 | 25.68 | 27.39 | 28.16 | 26.83 | 27.18 |
| 17 | 7 | 26.43 | 27.67 | 27.02 | 25.84 | 26.53 | 27.23 | 26.65 | 25.88 | 27.74 | 28.27 | 27.08 | 27.50 |
| 18 | 7 | 26.31 | 27.45 | 27.03 | 26.23 | 26.45 | 27.54 | 26.83 | 25.83 | 27.23 | 27.97 | 26.74 | 26.99 |
| 19 | 7 | 26.31 | 27.48 | 27.04 | 25.91 | Excluded | Excluded | Excluded | 25.91 | 27.45 | 28.06 | 26.71 | 27.29 |
| 20 | 7 | 26.32 | 27.41 | 26.84 | Excluded | Excluded | Excluded | Excluded | 26.17 | 27.34 | 28.16 | 26.72 | 27.03 |
| Mean | | 26.40 | 27.63 | 27.01 | 26.17 | 26.50 | 27.44 | 26.93 | 25.94 | 27.43 | 28.16 | 26.87 | 26.92 |
| 1 | 14 | 26.68 | 27.84 | 27.34 | 26.22 | 26.68 | 27.68 | 27.05 | 25.61 | 29.68 | 29.52 | 27.51 | 28.35 |
| 2 | 14 | 26.56 | 27.75 | 27.29 | 26.30 | 26.70 | 27.77 | 27.21 | 26.05 | 29.56 | 29.33 | 27.34 | 28.21 |
| 3 | 14 | 26.55 | 27.59 | 27.20 | 25.73 | 26.73 | 27.80 | 27.12 | 26.06 | 29.60 | 29.50 | 27.42 | 27.79 |
| 4 | 14 | 26.51 | 27.64 | 27.29 | 25.93 | 26.71 | 27.86 | 27.14 | 27.08 | 29.49 | 29.40 | 27.17 | 28.11 |
| 5 | 14 | 26.64 | 27.81 | 27.20 | 25.60 | 26.78 | 27.76 | 27.20 | 25.69 | 29.46 | 29.42 | 27.44 | 27.93 |
| 6 | 14 | 26.59 | 27.75 | 27.16 | 25.98 | 26.58 | 27.62 | 27.15 | 25.94 | 29.41 | 29.51 | 27.37 | 28.18 |
| 7 | 14 | 26.63 | 27.72 | 27.14 | 25.82 | 26.52 | 27.48 | 27.02 | 25.44 | 29.40 | 29.46 | 27.24 | 27.96 |
| 8 | 14 | 26.56 | 27.88 | 27.15 | 26.46 | 26.46 | 27.51 | 27.05 | 26.03 | 29.02 | 28.86 | 26.29 | 28.25 |
| 9 | 14 | 26.60 | 27.72 | 27.41 | 25.93 | 26.59 | 27.58 | 27.10 | 25.47 | 29.63 | 29.18 | 27.52 | 27.88 |
| 10 | 14 | 26.42 | 27.54 | 27.11 | 26.20 | 26.54 | 27.63 | 26.97 | 26.02 | 29.26 | 29.13 | 27.03 | 27.66 |
| 11 | 14 | 26.51 | 27.51 | 27.17 | 25.80 | 26.71 | 27.72 | 27.06 | 25.80 | 29.27 | 29.14 | 27.09 | 27.74 |
| 12 | 14 | 26.51 | 27.67 | 27.23 | 26.02 | 26.99 | 28.34 | 27.37 | 26.11 | 29.22 | 29.09 | 27.02 | 27.59 |
| 13 | 14 | 26.62 | 27.68 | 27.15 | 25.97 | 26.69 | 27.75 | 27.13 | 25.82 | 29.33 | 29.21 | 27.16 | 27.69 |
| 14 | 14 | 26.51 | 27.57 | 27.21 | 26.51 | 26.62 | 27.62 | 27.09 | 25.72 | 29.28 | 29.22 | 27.11 | 27.96 |
| 15 | 14 | 26.72 | 27.86 | 27.33 | 26.05 | 26.69 | 27.52 | 27.03 | 25.59 | 29.30 | 29.20 | 27.10 | 27.63 |
| 16 | 14 | Excluded | Excluded | Excluded | 26.25 | 26.55 | 27.64 | 26.97 | 25.89 | 29.41 | 29.32 | 27.15 | 28.05 |
| 17 | 14 | 26.58 | 27.71 | 27.18 | 26.18 | 26.56 | 27.68 | 27.14 | 25.72 | 29.17 | 28.75 | 26.86 | 27.60 |
| 18 | 14 | 26.48 | 27.59 | 27.39 | 26.34 | 26.63 | 27.66 | 27.20 | 25.85 | 28.79 | 28.04 | 25.64 | 27.45 |
| 19 | 14 | 26.42 | 27.42 | 27.11 | 26.03 | 26.75 | 27.84 | 27.22 | 26.04 | 29.29 | 29.29 | 26.41 | 27.53 |
| 20 | 14 | 26.51 | 27.67 | 27.14 | 26.14 | 27.10 | 28.36 | 27.53 | Excluded | 29.37 | 29.25 | 27.05 | 28.00 |
| Mean | | 26.56 | 27.68 | 27.22 | 26.07 | 26.68 | 27.74 | 27.14 | 25.89 | 29.35 | 29.19 | 27.05 | 27.88 |
| 1 | 30 | 27.19 | 28.12 | 28.42 | 26.17 | 26.80 | 27.67 | 28.16 | 25.55 | 33.31 | 31.31 | 28.47 | Excluded |
| 2 | 30 | 26.90 | 27.97 | 28.02 | 26.06 | 26.93 | 27.54 | 27.86 | 25.87 | 32.76 | 31.12 | 27.92 | 28.86 |
| 3 | 30 | 27.26 | 28.26 | 28.33 | Excluded | 27.07 | 28.09 | 28.36 | 25.98 | 33.12 | 31.21 | 27.89 | 29.02 |
| 4 | 30 | 26.95 | 28.02 | 27.84 | 25.97 | 26.97 | 27.87 | 28.15 | Excluded | 33.05 | 30.50 | 27.68 | 28.69 |
| 5 | 30 | 26.97 | 27.82 | 27.71 | 25.85 | 26.96 | 27.85 | 28.17 | 25.84 | 33.52 | 31.52 | 27.93 | 29.02 |
| 6 | 30 | 27.07 | 28.04 | 27.95 | 25.74 | 26.67 | 27.63 | 27.82 | 25.57 | 33.32 | 31.32 | 27.97 | 29.02 |
| 7 | 30 | 27.05 | 28.07 | 27.94 | 26.04 | 26.86 | 27.71 | 28.04 | 25.53 | 33.36 | 31.65 | 28.13 | 29.32 |
| 8 | 30 | 27.09 | 28.10 | 27.86 | 25.17 | 26.71 | 27.51 | 27.73 | 25.44 | 33.94 | 31.44 | 27.96 | 29.29 |
| 9 | 30 | 27.09 | 28.07 | 27.98 | 26.01 | 26.70 | 27.54 | 27.66 | 25.47 | 34.05 | 32.00 | 28.22 | 29.64 |
| 10 | 30 | 27.00 | 27.93 | 28.02 | 25.57 | 26.83 | 27.68 | 27.91 | 25.58 | 33.21 | 31.87 | 28.29 | 29.26 |
| 11 | 30 | 26.87 | 27.84 | 27.78 | 25.64 | 26.67 | 27.60 | 27.87 | 25.55 | 33.59 | 31.72 | 28.25 | 29.17 |
| 12 | 30 | 26.88 | 27.81 | 27.77 | 25.99 | 27.08 | 27.79 | 27.85 | 25.67 | 33.59 | 31.51 | 27.92 | 29.10 |
| 13 | 30 | 26.90 | 28.01 | 27.78 | 25.58 | 26.77 | 27.85 | 27.95 | 25.84 | 33.27 | 31.62 | 28.26 | 29.08 |
| 14 | 30 | 26.82 | 27.93 | 27.85 | 25.98 | 26.61 | 27.50 | 28.03 | 26.08 | 33.48 | 31.54 | 27.94 | 29.12 |
| 15 | 30 | 26.93 | 28.04 | 27.87 | 26.04 | 26.65 | 27.51 | 27.93 | 25.59 | 33.57 | 31.73 | 28.16 | 29.35 |
| 16 | 30 | 27.09 | 28.12 | 27.80 | Excluded | 26.50 | 27.46 | 27.71 | 25.56 | 34.32 | 31.60 | 27.95 | 29.23 |
| 17 | 30 | 27.19 | 28.16 | 28.32 | 26.30 | 26.71 | 27.52 | 27.77 | 25.66 | 33.06 | 31.17 | 27.03 | 28.95 |
| 18 | 30 | 27.03 | 28.03 | 28.31 | 26.19 | 26.67 | 27.68 | 27.73 | 25.65 | 32.62 | 31.26 | 28.06 | 29.24 |
| 19 | 30 | 26.96 | 28.05 | 28.07 | 26.23 | 26.69 | 27.64 | 27.78 | 25.56 | 33.08 | 31.38 | 28.05 | 29.20 |
| 20 | 30 | 26.99 | 27.91 | 28.02 | 26.27 | 26.96 | 28.03 | 28.09 | 26.77 | 34.48 | 31.56 | 28.11 | 29.20 |
| Mean | | 27.01 | 28.01 | 27.98 | 25.93 | 26.79 | 27.68 | 27.93 | 25.72 | 33.44 | 31.45 | 28.01 | 29.15 |
| 1 | 60 | 27.92 | 28.48 | 28.18 | 26.16 | 26.48 | 27.30 | 27.79 | 24.40 | 37.30 | 34.24 | 28.04 | 30.44 |
| 2 | 60 | 27.72 | 28.34 | 27.92 | 25.96 | 26.51 | 27.28 | 27.80 | 24.34 | 37.46 | 34.18 | 28.05 | 30.28 |
| 3 | 60 | 27.35 | 28.06 | 27.25 | 26.17 | 26.61 | 27.53 | 27.89 | 24.40 | 37.18 | 34.09 | 28.21 | 30.53 |
| 4 | 60 | 27.79 | 28.46 | 28.11 | 25.77 | 26.60 | 27.53 | 27.99 | Excluded | 0.00 | 34.08 | 28.07 | 30.29 |
| 5 | 60 | 27.85 | 28.47 | 28.13 | 25.69 | 26.71 | 27.69 | 28.14 | 24.52 | 42.45 | 34.07 | 28.13 | 30.26 |
| 6 | 60 | 27.39 | 27.96 | 27.23 | 25.26 | 26.62 | 27.54 | 27.98 | 24.65 | 35.72 | 34.05 | 27.91 | 30.39 |
| 7 | 60 | 27.43 | 27.98 | 27.23 | 25.83 | 26.51 | 27.39 | 27.87 | 24.39 | 36.62 | 34.08 | 28.24 | 30.38 |
| 8 | 60 | 27.36 | 27.77 | 27.06 | 25.50 | 26.46 | 27.33 | 27.79 | 24.28 | 37.77 | 34.53 | 28.19 | Excluded |
| 9 | 60 | 28.58 | 29.06 | 28.56 | 26.01 | 26.60 | 27.54 | 27.89 | 24.83 | 38.89 | 34.63 | 28.12 | Excluded |
| 10 | 60 | 27.80 | 28.26 | 28.01 | 25.70 | 26.42 | 27.29 | 27.76 | 24.95 | 38.60 | 34.07 | 28.08 | 30.09 |
| 11 | 60 | 27.75 | 28.29 | 28.08 | 25.56 | 26.66 | 27.67 | 28.13 | 24.93 | 36.16 | 35.01 | 27.84 | 30.17 |
| 12 | 60 | 27.78 | 28.38 | 28.07 | 25.35 | 26.62 | 27.55 | 27.76 | 24.90 | 35.98 | 33.80 | 27.90 | 30.19 |
| 13 | 60 | 27.69 | 28.23 | 28.01 | 25.57 | 26.68 | 27.61 | 28.00 | 25.44 | 37.46 | 34.21 | 27.86 | 30.01 |
| 14 | 60 | 27.77 | 28.29 | 27.97 | 25.43 | 26.63 | 27.58 | 27.82 | 25.05 | 36.53 | 33.53 | 27.86 | 30.06 |
| 15 | 60 | 27.89 | 28.35 | 28.02 | 25.79 | 26.53 | 27.39 | 27.81 | 24.79 | 38.26 | 34.16 | 28.01 | 30.26 |
| 16 | 60 | 27.86 | 28.43 | 28.10 | 25.48 | 26.51 | 27.49 | 27.76 | 24.79 | 35.99 | 34.29 | 27.94 | 30.37 |
| 17 | 60 | 27.83 | 28.49 | 28.05 | 25.82 | 26.46 | 27.44 | 27.95 | 24.74 | 35.29 | 34.50 | 27.80 | 30.73 |
| 18 | 60 | 27.87 | 28.37 | 28.17 | 25.73 | 26.53 | 27.41 | 28.01 | 24.64 | 37.91 | 34.43 | 28.02 | 30.23 |
| 19 | 60 | 27.57 | 28.16 | 28.03 | 25.14 | 26.62 | 27.55 | 27.76 | 24.63 | 39.82 | 34.23 | 28.11 | 30.20 |
| 20 | 60 | 27.55 | 28.17 | 27.81 | 25.43 | 26.78 | 27.72 | 28.06 | 24.97 | 36.74 | 33.93 | 27.88 | 30.17 |
| Mean | 60 | 27.74 | 28.30 | 27.90 | 25.67 | 26.58 | 27.49 | 27.90 | 24.72 | 37.48 | 34.21 | 28.01 | 30.28 |
| 1 | 90 | 29.46 | 30.50 | 28.28 | 28.21 | 26.90 | 28.21 | 28.04 | 26.50 | 35.22 | 36.65 | 28.68 | 29.07 |
| 2 | 90 | 29.36 | 30.17 | 27.84 | 27.52 | 26.84 | 28.20 | 28.01 | 26.82 | 34.40 | 36.53 | 27.88 | 29.82 |
| 3 | 90 | 29.32 | 30.19 | 27.91 | 27.69 | 26.84 | 28.07 | 27.99 | 26.89 | 35.03 | 35.97 | 27.78 | 29.31 |
| 4 | 90 | 29.37 | 30.13 | 27.81 | 27.74 | 27.07 | 28.43 | 28.13 | Excluded | 35.75 | 37.08 | 27.72 | 29.45 |
| 5 | 90 | 29.51 | 30.26 | 28.04 | 27.92 | 26.67 | 28.08 | 27.95 | 26.40 | 36.55 | 35.94 | 27.89 | 29.95 |
| 6 | 90 | NA | NA | NA | 27.64 | 26.52 | 27.84 | 27.61 | 26.21 | 35.31 | 36.37 | 27.84 | 29.74 |
| 7 | 90 | 29.28 | 30.18 | 27.90 | 27.87 | 26.62 | 27.90 | 27.58 | 26.21 | 35.90 | 35.93 | 28.12 | 29.80 |
| 8 | 90 | 29.36 | 30.15 | 27.88 | 27.64 | 26.36 | 27.65 | 27.52 | 26.09 | 35.12 | 36.42 | 28.13 | Excluded |
| 9 | 90 | 29.35 | 30.22 | 28.04 | 27.72 | 26.41 | 27.68 | 27.55 | 26.14 | ND | ND | ND | ND |
| 10 | 90 | 29.34 | 30.17 | 28.01 | 27.73 | 26.34 | 27.50 | 27.45 | 26.18 | ND | ND | ND | ND |
| 11 | 90 | 29.24 | 30.08 | 27.72 | 27.60 | 26.85 | 28.16 | 27.87 | 26.21 | ND | ND | ND | ND |
| 12 | 90 | 29.23 | 30.03 | 27.61 | 27.59 | 26.55 | 27.78 | 27.59 | 26.40 | ND | ND | ND | ND |
| 13 | 90 | 29.36 | 30.11 | 27.91 | 27.80 | 26.65 | 28.12 | 27.92 | Excluded | ND | ND | ND | ND |
| 14 | 90 | 29.15 | 30.05 | 28.02 | 27.48 | 26.48 | 27.76 | 27.40 | 26.17 | ND | ND | ND | ND |
| 15 | 90 | 29.33 | 30.27 | 28.06 | 27.48 | 26.39 | 27.65 | 27.69 | 26.20 | ND | ND | ND | ND |
| 16 | 90 | 29.28 | 30.36 | 28.07 | 28.04 | 26.42 | 27.76 | 27.45 | 26.26 | ND | ND | ND | ND |
| 17 | 90 | 29.43 | 30.36 | 28.18 | 28.04 | 26.36 | 27.59 | 27.39 | 26.23 | ND | ND | ND | ND |
| 18 | 90 | 29.29 | 30.25 | 28.12 | 27.61 | 26.54 | 27.68 | 27.97 | 26.18 | ND | ND | ND | ND |
| 19 | 90 | 29.33 | 30.19 | 27.94 | 27.48 | 26.52 | 27.58 | 27.65 | 26.25 | ND | ND | ND | ND |
| 20 | 90 | 29.20 | 30.14 | 28.01 | 27.72 | 26.74 | 27.99 | 27.85 | 26.41 | ND | ND | ND | ND |
| Mean | 90 | 29.33 | 30.20 | 27.97 | 27.72 | 26.60 | 27.88 | 27.73 | 26.32 | 35.41 | 36.36 | 28.00 | 29.59 |
| 1 | 120 | 30.72 | 30.15 | 27.28 | 28.43 | 26.20 | 27.44 | 27.06 | 25.08 | 36.02 | 36.76 | 27.35 | 30.53 |
| 2 | 120 | 29.30 | 29.20 | 27.10 | 27.07 | 26.17 | 27.41 | 26.64 | 25.01 | 35.40 | 38.01 | 27.26 | 30.22 |
| 3 | 120 | 29.32 | 29.19 | 27.00 | 27.05 | 26.18 | 27.42 | 26.64 | 25.10 | 34.37 | 36.60 | 27.18 | 30.07 |
| 4 | 120 | 29.30 | 29.20 | 26.99 | 26.93 | 26.28 | 27.49 | 26.73 | 25.00 | 35.07 | 37.34 | 27.51 | 30.23 |
| 5 | 120 | 29.25 | 29.22 | 26.82 | 26.58 | 26.36 | 27.79 | 27.41 | 27.18 | 34.72 | 36.89 | 27.29 | 30.29 |
| 6 | 120 | 28.99 | 28.70 | 26.91 | 26.93 | 26.32 | 27.67 | 27.42 | 26.92 | 35.03 | 38.39 | 27.14 | 30.19 |
| 7 | 120 | 28.93 | 28.57 | 26.91 | 27.07 | 26.30 | 27.64 | 27.37 | 26.80 | 34.89 | 36.78 | 27.20 | 30.58 |
| 8 | 120 | 28.98 | 28.53 | 25.51 | 26.79 | 26.16 | 27.50 | 27.13 | 26.67 | 36.80 | 36.58 | 27.27 | 30.38 |
| 9 | 120 | 29.37 | 29.41 | 27.35 | 26.82 | 26.18 | 27.38 | 27.06 | 26.43 | ND | ND | ND | ND |
| 10 | 120 | 29.21 | 29.19 | 26.88 | 26.89 | 26.20 | 27.42 | 27.18 | 27.08 | ND | ND | ND | ND |
| 11 | 120 | 29.11 | 29.16 | 26.88 | 27.04 | 26.28 | 27.51 | 27.03 | 26.64 | ND | ND | ND | ND |
| 12 | 120 | 29.16 | 29.08 | 26.75 | 27.10 | 26.25 | 27.60 | 26.86 | 26.30 | ND | ND | ND | ND |
| 13 | 120 | 29.21 | 29.30 | 27.06 | 26.91 | 26.35 | 27.77 | 27.15 | 25.95 | ND | ND | ND | ND |
| 14 | 120 | 29.04 | 29.06 | 26.76 | 26.87 | 26.22 | 27.67 | 26.94 | 26.25 | ND | ND | ND | ND |
| 15 | 120 | 29.26 | 29.24 | 27.01 | 27.03 | 26.21 | 27.45 | 27.05 | 26.32 | ND | ND | ND | ND |
| 16 | 120 | 29.17 | 29.30 | 27.01 | 27.19 | 26.18 | 27.36 | 26.86 | 26.31 | ND | ND | ND | ND |
| 17 | 120 | 29.42 | 29.45 | 27.13 | 27.35 | 26.12 | 27.31 | 26.79 | 25.67 | ND | ND | ND | ND |
| 18 | 120 | 29.27 | 29.26 | 27.08 | 27.14 | 26.12 | 27.33 | 26.89 | 25.93 | ND | ND | ND | ND |
| 19 | 120 | 29.14 | 29.15 | 27.04 | 26.84 | 26.18 | 27.42 | 26.84 | 25.83 | ND | ND | ND | ND |
| 20 | 120 | 29.33 | 29.24 | 27.02 | 27.09 | 26.38 | 27.58 | 27.04 | 24.29 | ND | ND | ND | ND |
| Mean | 120 | 29.27 | 29.18 | 26.93 | 27.06 | 26.23 | 27.51 | 27.00 | 26.04 | 35.29 | 37.17 | 27.27 | 30.31 |
| 1 | 150 | 32.67 | 33.16 | 27.68 | 29.91 | 26.94 | 28.55 | 27.64 | 28.57 | 36.20 | 38.22 | 27.39 | 31.17 |
| 2 | 150 | 32.33 | 32.48 | 27.48 | 30.01 | 26.77 | 28.42 | 27.30 | 28.56 | 38.19 | 44.17 | 27.59 | 32.19 |
| 3 | 150 | 37.57 | 36.04 | 29.30 | 35.17 | 26.70 | 28.44 | 27.25 | 28.34 | 37.13 | 37.40 | 27.41 | 31.06 |
| 4 | 150 | 32.65 | 32.51 | 27.47 | 29.73 | 26.66 | 28.34 | 27.22 | 28.24 | 37.89 | 37.24 | 27.28 | 31.13 |
| 5 | 150 | 31.79 | 32.13 | 27.23 | 30.11 | 26.66 | 28.29 | 27.13 | 28.27 | 42.93 | 36.99 | 27.20 | 31.03 |
| 6 | 150 | 31.96 | 32.21 | 27.19 | 29.75 | 26.58 | 28.29 | 27.17 | 28.00 | 40.48 | 36.42 | 27.26 | 31.14 |
| 7 | 150 | 31.79 | 32.23 | 27.29 | 29.69 | 26.65 | 28.34 | 27.13 | 28.19 | 0.00 | 0.00 | 28.62 | 35.19 |
| 8 | 150 | 32.15 | 32.42 | 27.32 | 29.55 | 26.69 | 28.38 | 27.11 | 28.17 | 35.19 | 37.18 | 27.43 | 30.94 |
| 9 | 150 | 32.42 | 32.72 | 27.44 | 29.51 | 26.72 | 28.33 | 27.18 | 29.01 | ND | ND | ND | ND |
| 10 | 150 | 32.07 | 32.30 | 27.37 | 29.32 | 26.66 | 28.27 | 27.23 | 28.56 | ND | ND | ND | ND |
| 11 | 150 | 32.20 | 32.25 | 27.26 | 29.47 | 26.66 | 28.34 | 27.22 | 28.32 | ND | ND | ND | ND |
| 12 | 150 | 31.74 | 32.03 | 27.14 | 29.65 | 26.70 | 28.34 | 27.18 | 28.33 | ND | ND | ND | ND |
| 13 | 150 | 32.16 | 32.51 | 27.31 | 30.20 | 26.51 | 28.17 | 27.16 | 28.28 | ND | ND | ND | ND |
| 14 | 150 | 32.54 | 32.53 | 27.24 | 29.81 | 26.52 | 28.23 | 27.23 | 28.41 | ND | ND | ND | ND |
| 15 | 150 | 32.94 | 32.78 | 27.27 | 29.98 | 26.47 | 28.19 | 27.04 | 28.23 | ND | ND | ND | ND |
| 16 | 150 | 32.26 | 32.82 | 27.43 | 29.99 | 26.62 | 28.25 | 27.28 | 28.06 | ND | ND | ND | ND |
| 17 | 150 | 32.63 | 32.77 | 27.50 | 29.97 | 26.89 | 28.54 | 27.57 | 28.95 | ND | ND | ND | ND |
| 18 | 150 | 32.52 | 32.80 | 27.58 | 29.80 | 30.04 | 31.23 | 29.08 | 31.79 | ND | ND | ND | ND |
| 19 | 150 | 32.20 | 32.65 | 27.44 | 29.66 | 26.72 | 28.36 | 27.43 | 28.69 | ND | ND | ND | ND |
| 20 | 150 | 31.99 | 32.52 | 27.39 | 29.77 | 26.69 | 28.40 | 27.31 | 28.73 | ND | ND | ND | ND |
| Mean | 150 | 32.53 | 32.69 | 27.47 | 30.05 | 26.84 | 28.49 | 27.34 | 28.58 | 38.29 | 38.23 | 27.52 | 31.73 |
| 1 | 180 | 32.90 | 32.06 | 27.64 | 28.61 | 26.67 | 28.18 | 27.58 | 26.85 | 35.96 | 39.01 | 28.00 | 30.46 |
| 2 | 180 | 32.62 | 32.05 | 27.75 | 28.61 | 26.67 | 28.15 | 27.55 | 26.92 | 35.62 | 37.61 | 27.84 | 30.22 |
| 3 | 180 | 32.21 | 32.10 | 27.55 | 28.42 | 26.64 | 28.08 | 27.50 | 26.52 | 35.73 | 37.22 | 28.01 | 30.04 |
| 4 | 180 | 32.36 | 32.25 | 27.53 | 28.73 | 26.54 | 27.96 | 27.29 | 27.07 | 35.65 | 37.54 | 27.53 | 30.25 |
| 5 | 180 | 32.17 | 32.02 | 27.92 | 28.86 | 26.64 | 28.09 | 27.45 | 26.79 | 37.05 | 37.53 | 27.59 | 29.95 |
| 6 | 180 | 32.38 | 32.20 | 27.68 | 28.46 | 26.53 | 27.89 | 27.41 | 26.48 | 36.03 | 37.79 | 27.50 | 29.64 |
| 7 | 180 | 32.18 | 31.87 | 27.74 | 28.38 | 26.53 | 27.96 | 27.37 | 26.62 | 36.99 | 37.45 | 27.54 | 29.77 |
| 8 | 180 | 32.35 | 31.74 | 27.74 | 28.37 | 26.61 | 28.13 | 27.45 | 26.68 | 35.61 | 36.69 | 27.54 | 30.10 |
| 9 | 180 | 32.12 | 31.59 | 27.42 | 28.40 | 26.75 | 28.26 | 27.69 | 27.29 | ND | ND | ND | ND |
| 10 | 180 | 32.19 | 31.77 | 27.51 | 28.24 | 26.63 | 28.08 | 27.56 | 26.57 | ND | ND | ND | ND |
| 11 | 180 | 32.35 | 31.71 | 27.40 | 28.36 | 26.68 | 28.12 | 27.54 | 26.67 | ND | ND | ND | ND |
| 12 | 180 | 32.07 | 31.61 | 27.49 | 28.57 | 26.55 | 28.05 | 27.42 | 26.97 | ND | ND | ND | ND |
| 13 | 180 | 32.81 | 32.16 | 28.09 | 28.94 | 26.53 | 28.02 | 27.67 | 28.17 | ND | ND | ND | ND |
| 14 | 180 | 32.63 | 32.15 | 27.80 | 28.81 | 26.49 | 28.02 | 27.44 | 26.56 | ND | ND | ND | ND |
| 15 | 180 | 32.52 | 32.10 | 27.91 | 28.66 | 26.47 | 27.99 | 27.36 | 26.71 | ND | ND | ND | ND |
| 16 | 180 | 32.60 | 31.94 | 27.82 | 28.92 | 26.50 | 28.01 | 27.40 | 26.85 | ND | ND | ND | ND |
| 17 | 180 | 32.26 | 32.13 | 27.87 | 28.33 | 26.64 | 28.17 | 27.66 | 27.48 | ND | ND | ND | ND |
| 18 | 180 | 32.26 | 31.87 | 27.64 | 28.48 | 26.60 | 28.13 | 27.57 | 27.02 | ND | ND | ND | ND |
| 19 | 180 | 32.38 | 31.86 | 27.46 | 28.56 | 26.61 | 28.09 | 27.54 | 26.86 | ND | ND | ND | ND |
| 20 | 180 | 32.30 | 32.01 | 27.60 | 28.82 | 26.51 | 27.99 | 27.36 | 26.57 | ND | ND | ND | ND |
| Mean | 180 | 32.38 | 31.96 | 27.68 | 28.58 | 26.59 | 28.07 | 27.49 | 26.88 | 36.08 | 37.60 | 27.69 | 30.05 |
| 1 | 210 | 26.68 | 27.89 | 27.63 | 31.43 | 33.72 | 33.12 | 28.44 | 28.44 | 36.16 | 37.43 | 27.90 | 32.75 |
| 2 | 210 | 26.75 | 27.95 | 27.61 | 31.31 | 33.17 | 32.54 | 28.12 | 28.32 | 36.13 | 37.30 | 27.78 | 32.46 |
| 3 | 210 | 26.70 | 27.96 | 27.71 | 31.00 | 33.29 | 32.72 | 28.00 | 28.57 | 34.47 | 37.54 | 28.03 | 32.44 |
| 4 | 210 | 26.96 | 28.07 | 27.71 | 31.00 | 33.14 | 32.48 | 28.08 | 28.58 | 35.84 | 36.59 | 27.76 | 32.23 |
| 5 | 210 | 33.17 | 32.45 | 28.12 | 30.66 | 26.96 | 28.13 | 27.93 | 28.84 | 35.93 | 43.76 | 27.89 | 32.32 |
| 6 | 210 | 32.65 | 32.26 | 28.03 | 30.83 | 26.80 | 28.03 | 27.86 | 29.33 | 36.03 | 38.65 | 27.89 | 32.31 |
| 7 | 210 | 33.22 | 32.40 | 27.87 | 30.26 | 27.08 | 28.15 | 28.11 | 28.89 | 37.24 | 37.27 | 28.07 | 32.39 |
| 8 | 210 | 33.09 | 32.39 | 27.88 | 30.50 | 26.84 | 28.04 | 27.77 | 28.56 | 37.60 | 38.29 | 28.21 | 32.47 |
| 9 | 210 | 33.00 | 32.28 | 28.00 | 30.41 | 26.85 | 28.13 | 27.95 | 28.72 | ND | ND | ND | ND |
| 10 | 210 | 32.96 | 32.37 | 27.68 | 30.45 | 26.56 | 27.71 | 27.63 | 28.42 | ND | ND | ND | ND |
| 11 | 210 | 33.39 | 32.69 | 28.06 | 30.46 | 26.65 | 27.81 | 27.61 | 28.61 | ND | ND | ND | ND |
| 12 | 210 | 33.21 | 32.31 | 27.91 | 30.93 | 26.57 | 27.80 | 27.58 | 28.71 | ND | ND | ND | ND |
| 13 | 210 | 33.46 | 32.53 | 28.07 | 30.57 | 26.65 | 28.01 | 27.76 | 28.97 | ND | ND | ND | ND |
| 14 | 210 | 33.19 | 32.30 | 27.92 | 30.34 | 26.81 | 28.01 | 27.73 | 29.22 | ND | ND | ND | ND |
| 15 | 210 | 33.87 | 32.54 | 28.01 | 30.55 | 26.83 | 28.02 | 27.80 | 28.56 | ND | ND | ND | ND |
| 16 | 210 | 33.05 | 32.32 | 27.63 | 30.71 | 26.98 | 28.16 | 27.85 | 28.86 | ND | ND | ND | ND |
| 17 | 210 | 33.44 | 32.21 | 27.80 | 30.69 | 26.77 | 28.00 | 27.74 | 28.56 | ND | ND | ND | ND |
| 18 | 210 | 33.03 | 32.41 | 27.90 | 31.23 | 26.70 | 27.96 | 27.79 | 29.18 | ND | ND | ND | ND |
| 19 | 210 | 33.08 | 32.27 | 27.80 | 30.72 | 26.69 | 27.96 | 27.75 | 28.74 | ND | ND | ND | ND |
| 20 | 210 | 34.17 | 32.21 | 28.00 | 30.59 | 26.71 | 28.01 | 27.82 | 28.64 | ND | ND | ND | ND |
| Mean | 210 | 31.95 | 31.49 | 27.87 | 30.73 | 28.09 | 28.94 | 27.87 | 28.74 | 36.18 | 38.36 | 27.94 | 32.42 |
| 1 | 270 | 34.96 | 34.11 | 28.21 | 31.26 | 27.38 | 29.16 | 28.56 | 29.05 | 32.39 | ND | 28.79 | 33.70 |
| 2 | 270 | 34.78 | 34.53 | 28.27 | 30.89 | 27.13 | 28.58 | 28.35 | 28.76 | 36.49 | 38.85 | 28.35 | 32.77 |
| 3 | 270 | 34.24 | 35.42 | 28.27 | 31.06 | 27.20 | 28.77 | 28.43 | 28.77 | 36.49 | 38.28 | 28.20 | 32.67 |
| 4 | 270 | 38.05 | 34.68 | 28.28 | 30.80 | 27.18 | 28.76 | 28.29 | 28.62 | 37.63 | 37.60 | 28.07 | 32.70 |
| 5 | 270 | 36.74 | 34.59 | 28.53 | 31.11 | 27.18 | 28.76 | 28.12 | 28.70 | 37.15 | 40.70 | 28.13 | 33.02 |
| 6 | 270 | 34.79 | 34.63 | 28.25 | 28.62 | 27.10 | 28.58 | 28.16 | 28.30 | 36.45 | 38.98 | 28.14 | 32.47 |
| 7 | 270 | 37.24 | 34.38 | 28.14 | 30.87 | 27.25 | 28.68 | 28.20 | 28.68 | 37.76 | 37.68 | 28.27 | 32.24 |
| 8 | 270 | 38.19 | 34.29 | 28.30 | 30.90 | 27.18 | 28.63 | 28.12 | 28.46 | 37.39 | 38.38 | 28.42 | 32.57 |
| 9 | 270 | 33.97 | 34.44 | 28.40 | 31.29 | 27.18 | 28.74 | 28.26 | 28.63 | ND | ND | ND | ND |
| 10 | 270 | 34.90 | 34.44 | 28.10 | 30.77 | 27.12 | 28.53 | 28.29 | 28.72 | ND | ND | ND | ND |
| 11 | 270 | 35.92 | 34.68 | 28.90 | 31.13 | 27.43 | 25.56 | 28.23 | 28.48 | ND | ND | ND | ND |
| 12 | 270 | 36.62 | 34.73 | 28.27 | 31.20 | 27.01 | 28.49 | 28.07 | 28.37 | ND | ND | ND | ND |
| 13 | 270 | 35.65 | 34.61 | 28.52 | 31.36 | 27.17 | 28.55 | 28.03 | 28.49 | ND | ND | ND | ND |
| 14 | 270 | 35.98 | 34.50 | 28.50 | 31.30 | 27.07 | 28.43 | 28.08 | 28.39 | ND | ND | ND | ND |
| 15 | 270 | 35.80 | 34.36 | 28.43 | 31.13 | 27.13 | 28.61 | 27.94 | 28.43 | ND | ND | ND | ND |
| 16 | 270 | 34.16 | 34.36 | 28.29 | 31.23 | 27.20 | 28.71 | 28.20 | 28.66 | ND | ND | ND | ND |
| 17 | 270 | 35.34 | 34.39 | 28.27 | 31.34 | 27.14 | 28.67 | 28.24 | 28.66 | ND | ND | ND | ND |
| 18 | 270 | 41.29 | 34.21 | 28.13 | 31.12 | 27.06 | 28.52 | 28.33 | 28.59 | ND | ND | ND | ND |
| 19 | 270 | 35.05 | 34.38 | 28.53 | 31.05 | 27.01 | 28.42 | 28.17 | 28.53 | ND | ND | ND | ND |
| 20 | 270 | 33.84 | 34.91 | 28.39 | 31.08 | 26.94 | 28.32 | 28.11 | 28.47 | ND | ND | ND | ND |
| Mean | 270 | 35.88 | 34.53 | 28.35 | 30.98 | 27.15 | 28.47 | 28.21 | 28.59 | 36.47 | 36.85 | 28.30 | 32.77 |

TABLE 15
Mean crossing thresholds from mocked clinical SARS-CoV-2 samples
spiked into DB#5 and stored up to 210 days at room temperature.
Data are represented as Mean (SD) of 20 replicates.

| Day | EGene | UTR | RNAseP | MS2 |
|---|---|---|---|---|
| 0 | 26.93 (0.41) | 27.78 (0.50) | 27.31 (0.25) | 26.03 (0.49) |
| 7 | 26.40 (0.15) | 27.63 (0.26) | 27.01 (0.14) | 26.17 (0.47) |
| 14 | 26.56 (0.08) | 27.68 (0.12) | 27.22 (0.09) | 26.07 (0.24) |
| 30 | 27.01 (0.12) | 28.02 (0.12) | 27.98 (0.21) | 25.93 (0.29) |
| 60 | 27.74 (0.27) | 28.30 (0.26) | 27.90 (0.39) | 25.67 (0.28) |
| 90 | 29.33 (0.09) | 30.20 (0.12) | 27.97 (0.16) | 27.73 (0.20) |
| 120 | 29.27 (0.37) | 29.18 (0.34) | 26.92 (0.37) | 27.06 (0.36) |
| 150 | 32.53 (1.23) | 32.69 (0.84) | 27.47 (0.45) | 30.05 (1.23) |
| 180 | 32.38 (0.23) | 31.96 (0.20) | 27.68 (0.19) | 28.58 (0.21) |
| 210 | 31.95 (2.68) | 31.49 (1.81) | 27.87 (0.16) | 30.73 (0.33) |

TABLE 16
Mean crossing thresholds from mocked clinical SARS-CoV-2
samples spiked into DB#5 and stored up to 210 days
at 4 C. Data are represented as Mean (SD) of 20 replicates.

| Day | EGene | UTR | RNAseP | MS2 |
|---|---|---|---|---|
| 0 | 26.93 (0.41) | 27.78 (0.50) | 27.31 (0.25) | 26.03 (0.49) |
| 7 | 26.50 (0.17) | 27.45 (0.27) | 26.93 (0.34) | 25.92 (0.48) |
| 14 | 26.69 (0.15) | 27.74 (0.23) | 27.19 (0.26) | 24.49 (6.25) |
| 30 | 26.77 (0.17) | 27.67 (0.20) | 27.91 (0.18) | 24.28 (6.20) |
| 60 | 26.60 (0.12) | 27.54 (0.20) | 27.91 (0.13) | 23.49 (6.02) |
| 90 | 26.57 (0.22) | 27.84 (0.27) | 27.68 (0.27) | 23.42 (8.70) |
| 120 | 26.27 (0.18) | 27.56 (0.27) | 27.03 (0.27) | 26.21 (0.95) |
| 150 | 26.83 (0.76) | 28.47 (0.66) | 27.34 (0.43) | 28.50 (0.89) |
| 180 | 26.94 (1.60) | 28.32 (1.13) | 27.53 (0.24) | 26.96 (0.53) |
| 210 | 27.79 (2.41) | 28.72 (1.72) | 27.84 (0.16) | 28.75 (0.27) |

TABLE 17
Mean crossing thresholds from mocked clinical SARS-CoV-2
samples spiked into DB#5 and stored up to 210 days
at 37 C. Data are represented as Mean (SD) of 20 replicates.

| Day | EGene | UTR | RNAseP | MS2 |
|---|---|---|---|---|
| 0 | 26.93 (0.41) | 27.78 (0.50) | 27.31 (0.25) | 26.03 (0.49) |
| 7 | 27.43 (0.12) | 28.16 (0.12) | 26.87 (0.14) | 26.92 (0.25) |
| 14 | 29.35 (0.21) | 29.19 (0.34) | 27.05 (0.46) | 27.88 (0.26) |
| 30 | 33.44 (0.48) | 31.45 (0.32) | 28.01 (0.29) | 27.59 (6.97) |
| 60 | 35.61 (8.54) | 34.21 (0.31) | 28.01 (0.13) | 27.05 (9.94) |
| 90 | 35.41 (0.65) | 36.36 (0.41) | 28.01 (0.31) | 25.64 (11.17) |
| 120 | 35.29 (0.78) | 37.17 (0.69) | 27.28 (0.12) | 30.31 (0.17) |
| 150 | 33.50 (13.76) | 33.45 (13.74) | 27.52 (0.46) | 31.73 (1.45) |
| 180 | 36.08 (0.60) | 37.61 (0.66) | 27.69 (0.22) | 30.05 (0.27) |
| 210 | 36.18 (0.95) | 38.35 (2.28) | 27.94 (0.15) | 32.42 (0.16) |

TABLE 18
shows the crossing thresholds from samples in freshly prepared DB#5 compared
to samples in DB#5 stored for up to 4 months; lots are indicated
by date of preparation and tested on Aug. 14, 2020.

| | | Mocked Sample | | | Mocked Sample | | |
|---|---|---|---|---|---|---|---|
| | Extraction | (24193 10-3) | | | (24193 10-4) | | |
| | Replicate | Tested Immediately | | | Tested Immediately | | |
| Lot # | # | E Gene | UTR | RNase P | E Gene | UTR | RNase P |
| Apr 25 | 1 | 24.48 | 25.35 | 24.32 | 28.01 | 28.63 | 24.67 |
| Apr 25 | 1 | 24.37 | 25.26 | 24.25 | 28.00 | 28.52 | 24.39 |
| Mean | | 24.42 | 25.31 | 24.29 | 28.01 | 28.58 | 24.53 |
| Apr 25 | 2 | 24.33 | 25.25 | 24.27 | 28.07 | 28.70 | 24.47 |
| Apr 25 | 2 | 24.28 | 25.19 | 24.28 | 28.14 | 28.79 | 24.58 |
| Mean | | 24.31 | 25.22 | 24.27 | 28.11 | 28.74 | 24.53 |
| May 10 | 1 | 24.34 | 25.33 | 24.19 | 27.71 | 28.33 | 24.11 |
| May 10 | 1 | 24.23 | 25.06 | 24.17 | 27.73 | 28.26 | 24.07 |
| Mean | | 24.29 | 25.20 | 24.18 | 27.72 | 28.29 | 24.09 |
| May 10 | 2 | 24.14 | 25.06 | 24.03 | 27.80 | 28.41 | 24.15 |
| May 10 | 2 | 24.09 | 25.05 | 24.06 | 28.04 | 28.99 | 24.39 |
| Mean | | 24.12 | 25.05 | 24.04 | 27.92 | 28.70 | 24.27 |
| June 11 | 1 | 24.38 | 25.50 | 24.71 | 27.97 | 28.44 | 24.25 |
| June 11 | 1 | 24.17 | 25.13 | 24.32 | 27.83 | 28.34 | 24.26 |
| Mean | | 24.28 | 25.31 | 24.51 | 27.90 | 28.39 | 24.26 |
| June 11 | 2 | 24.05 | 24.93 | 24.17 | 28.30 | 28.94 | 24.78 |
| June 11 | 2 | 24.03 | 24.84 | 24.00 | 28.09 | 28.67 | 24.48 |
| Mean | | 24.04 | 24.88 | 24.08 | 28.19 | 28.81 | 24.63 |
| June 23 | 1 | 24.28 | 25.33 | 24.30 | 27.94 | 28.47 | 24.32 |
| June 23 | 1 | 24.18 | 25.09 | 24.16 | 27.88 | 28.47 | 24.48 |
| Mean | | 24.23 | 25.21 | 24.23 | 27.91 | 28.47 | 24.40 |
| June 23 | 2 | 24.18 | 25.21 | 24.26 | 27.84 | 28.36 | 24.44 |
| June 23 | 2 | 24.05 | 25.06 | 24.17 | 28.05 | 28.66 | 24.79 |
| Mean | | 24.11 | 25.13 | 24.22 | 27.95 | 28.51 | 24.62 |
| July 4 | 1 | 24.29 | 25.34 | 24.46 | 27.71 | 28.40 | 24.50 |
| July 4 | 1 | 24.11 | 25.04 | 24.41 | 27.82 | 28.32 | 24.48 |
| Mean | | 24.20 | 25.19 | 24.44 | 27.77 | 28.36 | 24.49 |
| July 4 | 2 | 24.16 | 25.04 | 24.18 | 28.05 | 28.60 | 24.48 |
| July 4 | 2 | 24.09 | 25.03 | 24.20 | 28.10 | 28.72 | 24.69 |
| Mean | | 24.13 | 25.03 | 24.19 | 28.07 | 28.66 | 24.58 |
| July 21 | 1 | 24.39 | 25.38 | 24.32 | 27.81 | 28.38 | 24.23 |
| July 21 | 1 | 24.16 | 25.16 | 24.17 | 27.70 | 28.25 | 24.11 |
| Mean | | 24.27 | 25.27 | 24.24 | 27.76 | 28.32 | 24.17 |
| July 21 | 2 | 24.07 | 24.94 | 24.23 | 27.93 | 28.52 | 24.60 |
| July 21 | 2 | 24.08 | 24.92 | 24.22 | 28.03 | 28.60 | 24.43 |
| Mean | | 24.07 | 24.93 | 24.22 | 27.98 | 28.56 | 24.52 |
| July 28 | 1 | 23.61 | 24.45 | 24.39 | 27.07 | 27.50 | 24.25 |
| July 28 | 1 | 23.39 | 24.21 | 24.11 | 26.87 | 27.38 | 24.13 |
| Mean | | 23.50 | 24.33 | 24.25 | 26.97 | 27.44 | 24.19 |
| July 28 | 2 | 23.52 | 24.31 | 24.22 | 27.01 | 27.54 | 24.36 |
| July 28 | 2 | 23.27 | 24.08 | 23.98 | 27.14 | 27.68 | 24.46 |
| Mean | | 23.39 | 24.19 | 24.10 | 27.08 | 27.61 | 24.41 |
| July 28 NPS | 1 | 24.00 | 25.00 | 24.39 | 26.90 | 27.51 | 24.20 |
| July 28 NPS | 1 | 23.47 | 24.20 | 24.09 | 26.98 | 27.47 | 24.13 |
| Mean | | 23.73 | 24.60 | 24.24 | 26.94 | 27.49 | 24.16 |
| July 28 NPS | 2 | 23.54 | 24.30 | 24.06 | 27.13 | 27.70 | 24.37 |
| July 28 NPS | 2 | 23.56 | 24.32 | 24.16 | 27.20 | 27.70 | 24.35 |
| Mean | | 23.55 | 24.31 | 24.11 | 27.16 | 27.70 | 24.36 |
| July 28 Oral | 1 | 23.74 | 24.57 | 24.14 | 27.09 | 27.58 | 24.40 |
| July 28 Oral | 1 | 23.48 | 24.23 | 24.14 | 27.00 | 27.48 | 24.18 |
| Mean | | 23.61 | 24.40 | 24.14 | 27.05 | 27.53 | 24.29 |
| July 28 Oral | 2 | 23.72 | 24.52 | 24.23 | 27.13 | 27.70 | 24.51 |
| July 28 Oral | 2 | 23.62 | 24.35 | 24.17 | 27.30 | 27.93 | 24.92 |
| Mean | | 23.67 | 24.44 | 24.20 | 27.21 | 27.82 | 24.71 |
| | | Mocked Sample | | | Mocked Sample | | |
| | Extraction | (24193 10-3) | | | (24193 10-4) | | |
| | Replicate | Tested Post 72 hrs | | | Tested Post 72 hrs | | |
| Lot # | # | E Gene | UTR | RNase P | E Gene | UTR | RNase P |
| Apr 25 | 1 | 24.38 | 25.39 | 24.52 | 27.98 | 28.51 | 24.56 |
| Apr 25 | 1 | 24.37 | 25.33 | 24.45 | 27.82 | 28.41 | 24.42 |
| Mean | | 24.38 | 25.36 | 24.49 | 27.90 | 28.46 | 24.49 |
| Apr 25 | 2 | 24.33 | 25.29 | 24.46 | 28.03 | 28.46 | 24.61 |
| Apr 25 | 2 | 24.27 | 25.26 | 24.27 | 28.06 | 28.70 | 24.78 |
| Mean | | 24.30 | 25.27 | 24.37 | 28.05 | 28.58 | 24.70 |
| May 10 | 1 | 24.21 | 25.28 | 24.20 | 27.73 | 28.34 | 24.15 |
| May 10 | 1 | 24.12 | 25.07 | 24.13 | 27.60 | 28.29 | 24.23 |
| Mean | | 24.17 | 25.18 | 24.16 | 27.67 | 28.31 | 24.19 |
| May 10 | 2 | 23.92 | 24.88 | 23.90 | 27.89 | 28.50 | 24.36 |
| May 10 | 2 | 24.03 | 25.05 | 23.95 | 28.19 | 28.79 | 24.65 |
| Mean | | 23.97 | 24.96 | 23.92 | 28.04 | 28.64 | 24.51 |
| June 11 | 1 | 24.29 | 25.41 | 24.54 | 27.77 | 28.29 | 24.45 |
| June 11 | 1 | 24.13 | 25.14 | 24.33 | 28.05 | 28.53 | 24.59 |
| Mean | | 24.21 | 25.28 | 24.44 | 27.91 | 28.41 | 24.52 |
| June 11 | 2 | 23.98 | 24.93 | 24.25 | 28.30 | 29.07 | 24.97 |
| June 11 | 2 | 23.84 | 24.84 | 24.08 | 28.04 | 28.61 | 24.45 |
| Mean | | 23.91 | 24.88 | 24.16 | 28.17 | 28.84 | 24.71 |
| June 23 | 1 | 24.25 | 25.30 | 24.52 | 27.79 | 28.40 | 24.48 |
| June 23 | 1 | 24.15 | 25.15 | 24.39 | 27.90 | 28.45 | 24.44 |
| Mean | | 24.20 | 25.23 | 24.46 | 27.84 | 28.43 | 24.46 |
| June 23 | 2 | 24.15 | 25.11 | 24.50 | 27.86 | 28.26 | 24.34 |
| June 23 | 2 | 24.09 | 25.03 | 24.46 | 28.05 | 28.69 | 24.66 |
| Mean | | 24.12 | 25.07 | 24.48 | 27.95 | 28.48 | 24.50 |
| July 4 | 1 | 24.20 | 25.31 | 24.56 | 27.60 | 28.27 | 24.45 |
| July 4 | 1 | 24.10 | 25.08 | 24.46 | 27.63 | 28.26 | 24.42 |
| Mean | | 24.15 | 25.20 | 24.51 | 27.61 | 28.26 | 24.43 |
| July 4 | 2 | 23.86 | 24.85 | 24.28 | 28.06 | 28.55 | 24.62 |
| July 4 | 2 | 23.89 | 24.91 | 24.19 | 28.06 | 28.76 | 24.90 |
| Mean | | 23.88 | 24.88 | 24.24 | 28.06 | 28.66 | 24.76 |
| July 21 | 1 | 24.25 | 25.33 | 24.52 | 27.86 | 28.41 | 24.56 |
| July 21 | 1 | 24.17 | 25.16 | 24.49 | 27.70 | 28.41 | 24.54 |
| Mean | | 24.21 | 25.25 | 24.50 | 27.78 | 28.41 | 24.55 |
| July 21 | 2 | 23.97 | 25.02 | 24.17 | 27.97 | 28.58 | 24.51 |
| July 21 | 2 | 23.85 | 24.83 | 24.07 | 28.08 | 28.68 | 24.71 |
| Mean | | 23.91 | 24.92 | 24.12 | 28.02 | 28.63 | 24.61 |
| July 28 | 1 | 23.62 | 24.49 | 24.40 | 27.02 | 27.48 | 24.30 |
| July 28 | 1 | 23.41 | 24.20 | 24.09 | 26.90 | 27.37 | 24.17 |
| Mean | | 23.52 | 24.35 | 24.24 | 26.96 | 27.43 | 24.24 |
| July 28 | 2 | 23.44 | 24.26 | 24.14 | 27.05 | 27.48 | 24.34 |
| July 28 | 2 | 23.42 | 24.19 | 24.18 | 27.14 | 27.61 | 24.33 |
| Mean | | 23.43 | 24.22 | 24.16 | 27.09 | 27.55 | 24.34 |
| July 28 NPS | 1 | 23.71 | 24.46 | 24.27 | 27.04 | 27.52 | 24.19 |
| July 28 NPS | 1 | 23.40 | 24.19 | 24.11 | 27.03 | 27.40 | 24.25 |
| Mean | | 23.55 | 24.32 | 24.19 | 27.03 | 27.46 | 24.22 |
| July 28 NPS | 2 | 23.42 | 24.16 | 24.16 | 27.09 | 27.59 | 24.21 |
| July 28 NPS | 2 | 23.43 | 24.17 | 24.04 | 27.28 | 27.79 | 24.53 |
| Mean | | 23.43 | 24.16 | 24.10 | 27.18 | 27.69 | 24.37 |
| July 28 Oral | 1 | 23.81 | 24.58 | 24.37 | 27.19 | 27.71 | 24.27 |
| July 28 Oral | 1 | 23.71 | 24.52 | 24.38 | 27.08 | 27.51 | 24.36 |
| Mean | | 23.76 | 24.55 | 24.37 | 27.14 | 27.61 | 24.31 |
| July 28 Oral | 2 | 23.61 | 24.35 | 24.22 | 27.41 | 27.90 | 24.47 |
| July 28 Oral | 2 | 23.52 | 24.32 | 24.32 | 27.67 | 28.24 | 24.67 |
| Mean | | 23.57 | 24.33 | 24.27 | 27.54 | 28.07 | 24.57 |

| | Extraction | | | |
|---|---|---|---|---|
| | Replicate | | Neg | |
| Lot # | # | E Gene | UTR | RNase P |
| Apr 25 | 1 | | | |
| Apr 25 | 1 | | | |
| Mean | | | | |
| Apr 25 | 2 | | | |
| Apr 25 | 2 | | | |
| Mean | | | | |
| May 10 | 1 | | | |
| May 10 | 1 | | | |
| Mean | | | | |
| May 10 | 2 | | | |
| May 10 | 2 | | | |
| Mean | | | | |
| June 11 | 1 | | | |
| June 11 | 1 | | | |
| Mean | | | | |
| June 11 | 2 | | | |
| June 11 | 2 | | | |
| Mean | | | | |
| June 23 | 1 | 0.00 | 0.00 | 0.00 |
| June 23 | 1 | | | |
| Mean | | 0.00 | 0.00 | 0.00 |
| June 23 | 2 | 0.00 | 0.00 | 0.00 |
| June 23 | 2 | | | |
| Mean | | 0.00 | 0.00 | 0.00 |
| July 4 | 1 | | | |
| July 4 | 1 | | | |
| Mean | | | | |
| July 4 | 2 | | | |
| July 4 | 2 | | | |
| Mean | | | | |
| July 21 | 1 | 0.00 | 0.00 | 0.00 |
| July 21 | 1 | | | |
| Mean | | 0.00 | 0.00 | 0.00 |
| July 21 | 2 | 0.00 | 0.00 | 0.00 |
| July 21 | 2 | | | |
| Mean | | 0.00 | 0.00 | 0.00 |
| July 28 | 1 | 0.00 | 0.00 | 0.00 |
| July 28 | 1 | | | |
| Mean | | 0.00 | 0.00 | 0.00 |
| July 28 | 2 | 0.00 | 0.00 | 0.00 |
| July 28 | 2 | | | |
| Mean | | 0.00 | 0.00 | 0.00 |
| July 28 NPS | 1 | | | |
| July 28 NPS | 1 | | | |
| Mean | | | | |
| July 28 NPS | 2 | | | |
| July 28 NPS | 2 | | | |
| Mean | | | | |
| July 28 Oral | 1 | | | |
| July 28 Oral | 1 | | | |
| Mean | | | | |
| July 28 Oral | 2 | | | |
| July 28 Oral | 2 | | | |
| Mean | | | | |

TABLE 19
shows the crossing thresholds from samples in freshly prepared DB#5 compared
to samples in DB#5 stored for up to 6 months or longer; lots are
indicated by date of preparation and tested on Nov. 8, 2020.

| | | Mocked Specimen | | | Mocked Specimen | | |
|---|---|---|---|---|---|---|---|
| | Ext | (CoV448 10-4) | | | (CoV 448 10-5) | | |
| Lot # | Rep # | E Gene | UTR | RNase P | E Gene | UTR | RNase P |
| Apr 25 | 1 | 27.46 | 28.71 | 26.34 | 31.01 | 32.07 | 25.65 |
| May 10 | 1 | 27.06 | 28.66 | 25.89 | 31.15 | 32.19 | 25.79 |
| June 11 | 1 | 28.10 | 29.39 | 26.23 | 29.61 | 30.58 | 25.83 |
| June 23 | 1 | 27.40 | 28.96 | 26.07 | 30.82 | 32.17 | 25.80 |
| July 4 | 1 | 27.50 | 28.81 | 25.89 | 31.72 | 32.59 | 26.24 |
| July 21 | 1 | 27.57 | 29.04 | 25.73 | 31.10 | 32.34 | 25.80 |
| July 28 | 1 | 27.04 | 28.36 | 25.78 | 31.01 | 32.13 | 25.72 |
| Aug 6 | 1 | 26.83 | 28.38 | 25.88 | 31.36 | 32.46 | 25.61 |
| Aug 26 | 1 | 27.73 | 29.09 | 26.20 | 31.24 | 32.34 | 25.79 |
| (Sept 2020) | 1 | 27.04 | 28.48 | 25.75 | 31.09 | 32.35 | 25.63 |
| 340-3071 | | | | | | | |
| 342-3071 | 1 | 27.73 | 29.03 | 25.79 | 31.08 | 32.25 | 25.59 |
| (Oct 2020) | | | | | | | |
| 344-3071 | 1 | 26.77 | 28.18 | 25.42 | 31.08 | 32.45 | 25.54 |
| 354-3071 | 1 | 27.46 | 29.00 | 25.53 | 31.13 | 32.45 | 25.92 |
| 356-3071 | 1 | 27.20 | 28.56 | 25.71 | 31.12 | 32.61 | 25.68 |
| 359-3071 | 1 | 27.29 | 28.70 | 25.67 | 29.18 | 30.82 | 25.63 |
| Mean | | 27.34 | 28.76 | 25.86 | 30.90 | 32.08 | 25.75 |
| (Oct 2020) | 1 | 27.72 | 29.03 | 26.40 | 30.89 | 32.19 | 26.18 |
| 362-3071 | | | | | | | |
| 362-3071 | 2 | 27.70 | 29.10 | 26.38 | 30.51 | 32.11 | 25.62 |
| Mean | | 27.71 | 29.07 | 26.39 | 30.70 | 32.15 | 25.90 |
| 599756 | 1 | 26.80 | 28.21 | 26.07 | 31.06 | 32.23 | 26.01 |
| 599756 | 2 | 27.07 | 28.37 | 26.31 | 31.02 | 32.21 | 25.85 |
| Mean | | 26.94 | 28.29 | 26.19 | 31.04 | 32.22 | 25.93 |

| | | Mocked Specimen | | |
|---|---|---|---|---|
| | Ext | (CoV448 10-4) | | |
| Lot # | Rep # | E Gene | UTR | RNase P |
| 362-3071 | 1 | 0.00 | 0.00 | 0.00 |
| (Oct 2020) | 2 | 0.00 | 0.00 | 0.00 |
| | 3 | 0.00 | 0.00 | 0.00 |
| | 4 | 0.00 | 0.00 | 0.00 |
| 599756 | 1 | 0.00 | 0.00 | 0.00 |
| | 2 | 0.00 | 0.00 | 0.00 |
| | 3 | 0.00 | 0.00 | 0.00 |
| | 4 | 0.00 | 0.00 | 0.00 |

Abbreviations: Ext Rep, Extraction replicate.

TABLE 20
crossing thresholds from samples prepared in DB#5 with or without
the addition of DTT and with or without the addition of BSA.

MMM Jun. 23, 2020 Lot

| | | | | |
|---|---|---|---|---|
| Day # | E Gene | UTR | RNase P | MS2 |

Room Temperature Storage

| | | | | |
|---|---|---|---|---|
| 0 | 30.53 | 31.17 | 27.48 | 26.76 |
| 0 | 30.34 | 31.31 | 27.16 | 26.53 |
| 0 | 30.41 | 31.29 | 27.28 | 26.38 |
| 0 | 30.69 | 31.26 | 27.38 | 26.75 |
| 0 | 30.32 | 31.38 | 27.21 | 26.12 |
| 0 | 30.65 | 31.22 | 27.24 | 26.52 |
| 0 | 30.55 | 31.36 | 27.29 | 26.26 |
| 0 | 30.78 | 31.33 | 27.24 | 25.94 |
| Mean | 30.54 | 31.29 | 27.28 | 26.41 |
| 3 | 30.23 | 31.14 | 27.70 | 25.28 |
| 3 | 30.14 | 31.08 | 27.84 | 25.21 |
| 3 | 30.46 | 31.12 | 27.63 | 25.46 |
| 3 | 30.14 | 31.06 | 27.43 | 24.96 |
| 3 | 30.09 | 30.91 | 27.45 | 25.16 |
| 3 | 30.23 | 31.07 | 27.39 | 24.43 |
| 3 | 30.18 | 31.12 | 27.61 | 25.09 |
| 3 | 30.27 | 31.15 | 27.67 | 25.06 |
| Mean | 30.22 | 31.08 | 27.59 | 25.08 |
| 7 | 30.12 | 30.51 | 27.27 | 25.45 |
| 7 | 30.10 | 30.29 | 27.02 | 25.13 |
| 7 | 29.77 | 30.23 | 26.95 | 25.36 |
| 7 | 29.98 | 30.26 | 26.81 | 25.25 |
| 7 | 30.10 | 30.34 | 26.95 | 25.25 |
| 7 | 30.10 | 30.16 | 27.01 | 25.10 |
| 7 | 30.14 | 30.37 | 27.08 | 24.81 |
| 7 | 30.04 | 30.38 | 27.08 | 24.61 |
| Mean | 30.04 | 30.32 | 27.02 | 25.12 |

4 C. Storage

| | | | | |
|---|---|---|---|---|
| 3 | 30.54 | 31.06 | 27.62 | Excluded |
| 3 | 30.31 | 30.99 | 27.52 | 25.47 |
| 3 | 30.24 | 31.01 | 27.46 | 25.42 |
| 3 | 30.29 | 31.09 | 27.47 | 25.10 |
| 3 | 30.54 | 31.27 | 27.33 | 25.54 |
| 3 | 30.42 | 30.97 | 27.61 | 25.27 |
| 3 | 30.35 | 31.20 | 27.42 | 25.55 |
| 3 | 30.29 | 31.14 | 27.59 | 25.69 |
| Mean | 30.37 | 31.09 | 27.50 | 25.44 |
| 7 | 30.60 | 30.66 | 27.38 | 24.85 |
| 7 | 30.17 | 30.44 | 26.94 | 24.83 |
| 7 | 30.18 | 30.42 | 26.75 | 24.58 |
| 7 | 30.24 | 30.51 | 27.18 | 26.06 |
| 7 | 30.20 | 30.28 | 26.94 | 24.58 |
| 7 | 30.19 | 30.37 | 26.87 | 24.46 |
| 7 | 30.12 | 30.37 | 26.87 | 24.65 |
| 7 | 30.45 | 30.73 | 27.27 | 24.70 |
| Mean | 30.27 | 30.47 | 27.03 | 24.84 |

MMM + 0.05 mg/ml BSA

| | | | | |
|---|---|---|---|---|
| Day # | E Gene | UTR | RNase P | MS2 |

Room Temperature Storage

| | | | | |
|---|---|---|---|---|
| 0 | 30.49 | 31.38 | 27.31 | 26.02 |
| 0 | 30.33 | 31.36 | 27.34 | 26.46 |
| 0 | 30.20 | 31.13 | 27.08 | 25.84 |
| 0 | 30.39 | 31.35 | 27.14 | 26.17 |
| 0 | 30.42 | 31.33 | 27.17 | 26.23 |
| 0 | 30.33 | 31.14 | 27.22 | 26.30 |
| 0 | 30.41 | 31.32 | 27.27 | 26.05 |
| 0 | 30.34 | 31.29 | 27.19 | 26.55 |
| Mean | 30.36 | 31.29 | 27.22 | 26.20 |
| 3 | 30.16 | 30.96 | 27.38 | 25.63 |
| 3 | 29.96 | 30.85 | 27.43 | 25.40 |
| 3 | 29.73 | 30.81 | 27.31 | 25.44 |
| 3 | 29.85 | 30.61 | 27.30 | 25.45 |
| 3 | 29.84 | 30.81 | 27.25 | 25.32 |
| 3 | 29.84 | 30.88 | 27.44 | 25.43 |
| 3 | 30.07 | 31.00 | 27.37 | 25.31 |
| 3 | 30.07 | 30.77 | 27.52 | 25.30 |
| Mean | 29.94 | 30.84 | 27.38 | 25.41 |
| 7 | 29.89 | 30.32 | 26.86 | 25.41 |
| 7 | 29.85 | 30.24 | 26.97 | 25.56 |
| 7 | 29.69 | 30.21 | 26.87 | 25.45 |
| 7 | 29.68 | 30.15 | 26.88 | 25.34 |
| 7 | 29.64 | 30.15 | 26.77 | 25.34 |
| 7 | 29.64 | 30.20 | 26.97 | 25.53 |
| 7 | 29.78 | 30.19 | 27.06 | 25.59 |
| 7 | Excluded | Excluded | Excluded | 25.31 |
| Mean | 29.78 | 30.21 | 26.91 | 25.44 |

4 C. Storage

| | | | | |
|---|---|---|---|---|
| 3 | 30.42 | 31.16 | 27.41 | 25.66 |
| 3 | 30.16 | 30.90 | 27.23 | 25.57 |
| 3 | 30.17 | 30.90 | 27.32 | 25.63 |
| 3 | 30.06 | 30.56 | 27.16 | 25.24 |
| 3 | 30.23 | 30.90 | 27.41 | 25.30 |
| 3 | 30.09 | 30.96 | 27.29 | 25.59 |
| 3 | 30.01 | 31.06 | 27.23 | 25.32 |
| 3 | 30.27 | 30.99 | 27.30 | 25.66 |
| Mean | 30.18 | 30.93 | 27.29 | 25.50 |
| 7 | 30.29 | 30.46 | 26.84 | 24.93 |
| 7 | 30.10 | 30.29 | 26.72 | 25.04 |
| 7 | 29.72 | 30.08 | 26.64 | 24.61 |
| 7 | 30.13 | 30.23 | 26.81 | 24.89 |
| 7 | 29.87 | 30.12 | 26.74 | 24.78 |
| 7 | 30.01 | 30.31 | 26.92 | 24.98 |
| 7 | 30.00 | 30.38 | 26.73 | 24.70 |
| 7 | 30.08 | 30.42 | 26.95 | 24.94 |
| Mean | 30.03 | 30.29 | 26.79 | 24.86 |

MMM + 0.1 mg/ml BSA

| | | | | |
|---|---|---|---|---|
| Day # | E Gene | UTR | RNase P | MS2 |

Room Temperature Storage

| | | | | |
|---|---|---|---|---|
| 0 | 30.69 | 31.62 | 27.38 | 27.29 |
| 0 | 30.29 | 31.14 | 27.07 | 26.71 |
| 0 | 30.44 | 31.20 | 27.01 | 26.29 |
| 0 | 30.29 | 31.21 | 26.86 | 26.60 |
| 0 | 30.49 | 31.11 | 27.08 | 26.66 |
| 0 | 30.28 | 31.21 | 26.85 | 26.20 |
| 0 | 30.48 | 31.22 | 27.06 | 26.98 |
| 0 | 30.46 | 31.33 | 27.21 | 26.95 |
| Mean | 30.43 | 31.25 | 27.07 | 26.71 |
| 3 | 30.21 | 31.05 | 27.41 | 26.54 |
| 3 | 29.95 | 31.10 | 27.41 | 26.14 |
| 3 | 29.97 | 30.76 | 27.43 | 25.91 |
| 3 | 30.10 | 30.88 | 27.47 | 26.00 |
| 3 | 30.57 | 31.54 | 27.46 | 27.33 |
| 3 | 29.74 | 30.65 | 27.34 | 25.92 |
| 3 | 30.12 | 31.19 | 27.69 | 26.67 |
| 3 | 30.07 | 30.90 | 27.52 | 26.03 |
| Mean | 30.09 | 31.01 | 27.47 | 26.32 |
| 7 | 30.10 | 30.33 | 27.14 | 26.20 |
| 7 | 29.86 | 30.33 | 27.03 | 26.01 |
| 7 | 30.66 | 30.24 | 26.57 | 26.59 |
| 7 | 29.66 | 30.19 | 26.52 | 26.15 |
| 7 | 29.80 | 30.21 | 26.75 | 26.03 |
| 7 | 30.01 | 30.14 | 26.87 | 25.97 |
| 7 | 29.77 | 30.35 | 26.74 | 25.96 |
| 7 | 30.11 | 30.45 | 26.71 | 25.41 |
| Mean | 30.00 | 30.28 | 26.79 | 26.04 |

4 C. Storage

| | | | | |
|---|---|---|---|---|
| 3 | 30.03 | 30.88 | 27.03 | 26.04 |
| 3 | 29.98 | 30.83 | 26.99 | 25.60 |
| 3 | 30.04 | 30.90 | 27.08 | 25.68 |
| 3 | 30.21 | 30.94 | 27.26 | 26.01 |
| 3 | 30.00 | 30.60 | 27.05 | 25.88 |
| 3 | 30.02 | 30.66 | 27.21 | 25.88 |
| 3 | 30.41 | 31.29 | 27.42 | 25.62 |
| 3 | 30.28 | 31.09 | 27.27 | 25.71 |
| Mean | 30.12 | 30.90 | 27.16 | 25.80 |
| 7 | 30.15 | 30.31 | 26.72 | 25.06 |
| 7 | 30.07 | 30.25 | 26.48 | 25.20 |
| 7 | 30.10 | 30.24 | 26.75 | 25.06 |
| 7 | 30.03 | 30.22 | 26.74 | 25.13 |
| 7 | 30.37 | 30.64 | 28.53 | 24.83 |
| 7 | 30.02 | 30.15 | 26.56 | 25.62 |
| 7 | 30.11 | 30.44 | 27.48 | 25.34 |
| 7 | 30.14 | 30.38 | 27.04 | 25.16 |
| Mean | 30.12 | 30.33 | 25.19 | 25.18 |

MMM + 0.25 mM DTT

| | | | | |
|---|---|---|---|---|
| Day # | E Gene | UTR | RNase P | MS2 |

Room Temperature Storage

| | | | | |
|---|---|---|---|---|
| 0 | 30.38 | 31.39 | 25.52 | 26.66 |
| 0 | 30.23 | 31.17 | 25.18 | 26.25 |
| 0 | 30.25 | 31.25 | 25.10 | 25.46 |
| 0 | 30.33 | 31.24 | 25.25 | 26.05 |
| 0 | 30.43 | 31.24 | 25.25 | 25.67 |
| 0 | 30.29 | 31.10 | 26.35 | 26.23 |
| 0 | 30.26 | 31.20 | 26.31 | 26.03 |
| 0 | 30.46 | 31.30 | 25.23 | 26.48 |
| Mean | 30.33 | 31.23 | 25.53 | 26.10 |
| 3 | 30.33 | 31.10 | 25.41 | 25.56 |
| 3 | 30.16 | 31.05 | 25.50 | 25.06 |
| 3 | 30.08 | 30.91 | 25.54 | 25.21 |
| 3 | 29.97 | 30.76 | 25.46 | 25.14 |
| 3 | 30.08 | 30.91 | 25.31 | 25.08 |
| 3 | 30.11 | 30.86 | 25.38 | 25.19 |
| 3 | 30.06 | 30.98 | 25.36 | 25.02 |
| 3 | 30.05 | 31.04 | 25.44 | 25.17 |
| Mean | 30.11 | 30.95 | 25.43 | 25.18 |
| 7 | 29.87 | 30.42 | 25.03 | 24.62 |
| 7 | 29.81 | 30.29 | 24.82 | 24.38 |
| 7 | 29.76 | 30.17 | 24.74 | 24.54 |
| 7 | 29.68 | 30.00 | 24.77 | 24.70 |
| 7 | 29.77 | 30.08 | 24.64 | 24.46 |
| 7 | 29.70 | 30.07 | 24.76 | 24.43 |
| 7 | 29.75 | 30.11 | 24.83 | 24.57 |
| 7 | 30.02 | 30.37 | 24.88 | 24.09 |
| Mean | 29.80 | 30.19 | 24.81 | 24.47 |

4 C. Storage

| | | | | |
|---|---|---|---|---|
| 3 | 30.31 | 31.03 | 25.63 | 25.79 |
| 3 | 30.08 | 31.04 | 25.57 | 25.13 |
| 3 | 30.10 | 30.81 | 25.44 | 25.11 |
| 3 | 30.20 | 31.06 | 25.42 | 25.19 |
| 3 | 30.13 | 30.84 | 25.43 | 25.47 |
| 3 | 30.09 | 30.94 | 25.32 | 25.12 |
| 3 | 30.20 | 30.91 | 25.50 | 25.18 |
| 3 | 30.29 | 31.14 | 25.44 | 25.35 |
| Mean | 30.17 | 30.97 | 25.47 | 25.29 |
| 7 | 30.21 | 30.37 | 25.19 | 24.87 |
| 7 | 30.15 | 30.33 | 25.13 | 25.00 |
| 7 | 30.14 | 30.26 | 25.18 | 25.05 |
| 7 | 29.89 | 30.28 | 25.10 | 24.98 |
| 7 | 30.04 | 30.15 | 24.90 | 24.94 |
| 7 | 30.16 | 30.14 | 25.17 | 26.62 |
| 7 | 30.23 | 30.24 | 25.03 | 25.15 |
| 7 | 30.38 | 30.62 | 25.12 | 24.94 |
| Mean | 30.15 | 30.30 | 25.10 | 25.19 |

MMM + 1 mM DTT

| | | | | |
|---|---|---|---|---|
| Day # | E Gene | UTR | RNase P | MS2 |

Room Temperature Storage

| | | | | |
|---|---|---|---|---|
| 0 | 30.72 | 31.22 | 26.95 | 26.35 |
| 0 | 30.27 | 31.13 | 27.05 | 26.62 |
| 0 | 30.41 | 31.29 | 27.02 | 26.57 |
| 0 | 30.33 | 31.22 | 27.04 | 26.58 |
| 0 | 30.29 | 31.25 | 26.95 | 27.00 |
| 0 | 30.40 | 31.21 | 26.77 | 26.67 |
| 0 | 30.53 | 31.35 | 27.11 | 26.79 |
| 0 | 30.52 | 31.58 | 27.25 | 27.12 |
| Mean | 30.43 | 31.28 | 27.02 | 26.71 |
| 3 | 30.17 | 31.04 | 27.05 | 25.45 |
| 3 | 29.90 | 30.71 | 26.84 | 25.38 |
| 3 | 29.85 | 30.76 | 26.94 | 25.25 |
| 3 | 29.90 | 30.68 | 26.93 | 25.26 |
| 3 | 29.98 | 30.68 | 27.04 | 25.10 |
| 3 | 29.88 | 30.55 | 27.00 | 25.30 |
| 3 | 30.08 | 30.98 | 26.94 | 25.29 |
| 3 | 29.84 | 30.35 | 26.95 | 25.63 |
| Mean | 29.95 | 30.72 | 26.96 | 25.33 |
| 7 | 30.07 | 30.29 | 26.85 | 24.95 |
| 7 | 29.63 | 30.07 | 26.33 | 24.94 |
| 7 | 29.78 | 30.12 | 26.32 | 24.74 |
| 7 | 29.52 | 29.89 | 26.28 | 24.93 |
| 7 | 29.75 | 30.15 | 26.56 | 24.64 |
| 7 | 29.71 | 30.04 | 26.21 | 24.86 |
| 7 | 29.69 | 30.20 | 26.38 | 25.07 |
| 7 | 30.08 | 30.53 | 26.76 | Excluded |
| Mean | 29.78 | 30.16 | 26.46 | 24.87 |

4 C. Storage

| | | | | |
|---|---|---|---|---|
| 3 | 30.13 | 30.82 | 26.98 | 25.22 |
| 3 | 30.00 | 30.68 | 26.79 | 25.16 |
| 3 | 29.86 | 30.78 | 26.87 | 24.90 |
| 3 | 30.01 | 30.88 | 26.99 | 25.08 |
| 3 | 29.89 | 30.78 | 26.90 | 25.36 |
| 3 | 30.01 | 30.79 | 27.05 | 25.06 |
| 3 | 29.87 | 30.69 | 26.88 | 25.10 |
| 3 | 30.07 | 30.67 | 26.67 | 25.52 |
| Mean | 29.98 | 30.76 | 26.89 | 25.17 |
| 7 | 30.14 | 30.24 | 26.58 | 24.81 |
| 7 | 30.08 | 30.15 | 26.36 | 24.78 |
| 7 | 29.99 | 30.24 | 26.33 | 24.67 |
| 7 | 30.01 | 30.24 | 26.50 | 24.84 |
| 7 | 29.79 | 30.12 | 26.43 | 24.59 |
| 7 | 29.99 | 30.13 | 26.48 | 24.87 |
| 7 | 29.87 | 30.31 | 26.49 | 24.74 |
| 7 | 30.06 | 30.44 | 26.57 | 24.41 |
| Mean | 29.99 | 30.23 | 26.47 | 24.71 |

TABLE 21
crossing thresholds from bead volume titration experiments.

| | COVID Triplex PCR | | |
|---|---|---|---|
| | Crossing Threshold (E gene/UTR) | | |
| | 5 μl Beads | 17.5 μl Beads | 35 μl Beads |
| 24193 10⁻³ | 29.45/29.94 | 27.63/28.24 | 26.68/27.21 |
| | 29.33/29.81 | 27.45/28.04 | 26.58/27.07 |
| | 29.31/29.55 | 27.43/27.87 | 26.40/26.90 |
| 24193 10⁻⁴ | 31.12/31.32 | 30.26/30.45 | 28.32/28.92 |
| | 30.86/31.12 | 29.76/30.12 | 28.22/28.72 |
| | 30.81/31.12 | 29.65/29.90 | 28.14/28.61 |
| 24193 10⁻⁵ | 35.52/35.65 | 34.75/34.37 | 33.06/32.90 |
| | 34.84/35.50 | 34.59/34.26 | 32.80/33.07 |
| | 36.28/35.48 | 33.90/34.05 | 32.94/32.56 |

TABLE 22
crossing thresholds from mixing time and speed experiments.

COVID Triplex PCR
Crossing Threshold (E gene/UTR)

| | VR 24193 10⁻⁴ | VR 24193 10⁻⁵ |
|---|---|---|
| 5 μl beads, | 32.12/32.24 | 35.67/35.46 |
| 15 mins mixing | 32.12/32.17 | 35.16/34.74 |
| 5 μl beads, | 32.56/32.25 | 35.58/35.31 |
| 22 mins mixing | 32.46/32.12 | 36.33/35.93 |
| 5 μl beads, | 32.25/32.43 | 37.12/35.81 |
| 30 mins mixing | 32.32/32.36 | 37.48/35.71 |
| 17.5 μl beads, | 30.70/31.05 | 34.08/34.37 |
| 15 mins mixing | 30.65/31.07 | 33.52/33.63 |
| 17.5 μl beads, | 30.89/31.08 | 34.27/34.30 |
| 22 mins mixing | 30.80/31.03 | 33.94/34.55 |
| 17.5 μl beads, | 31.31/31.37 | 33.25/33.86 |
| 30 mins mixing | 31.26/31.45 | 34.12/33.82 |
| 35 μl beads, | 30.01/30.30 | 33.55/33.63 |
| 15 mins mixing | 29.92/30.25 | 33.40/34.19 |
| 35 μl beads, | 30.01/30.27 | 33.53/33.36 |
| 22 mins mixing | 30.05/30.44 | 33.23/33.23 |
| 35 μl beads, | 30.77/30.81 | 33.81/33.56 |
| 30 mins mixing | 30.76/30.82 | 33.47/34.23 |

TABLE 23
crossing thresholds from experiments omitting lysis buffer.

COVID Triplex PCR
Crossing Threshold (E gene/UTR)

| | With Lysis Buffer | No Lysis Buffer |
|---|---|---|
| VR 24193 10⁻⁴, | 33.16/33.21 | 32.05/31.84 |
| 5 μl beads | 32.57/33.07 | 32.13/31.77 |
| | Mean = 32.87/33.14 | Mean = 32.09/31.80 |
| VR 24193 10⁻⁴, | 31.06/31.31 | 30.50/30.00 |
| 17.5 μl beads | 31.27/31.32 | 30.36/30.22 |
| | Mean = 31.16/31.32 | Mean = 30.43/30.11 |
| VR 24193 10⁻⁴, | 30.18/30.24 | 30.02/29.72 |
| 35 μl beads | 30.43/30.29 | 29.78/29.57 |
| | Mean = 30.31/30.27 | Mean = 29.90/29.65 |
| VR 24193 10⁻⁵, | 38.80/37.01 | 37.27/36.13 |
| 5 μl beads | 37.60/38.26 | 36.12/36.93 |
| | Mean = 38.20/37.63 | Mean = 36.70/36.53 |
| VR 24193 10⁻⁵, | 34.62/33.47 | 34.83/35.23 |
| 17.5 μl beads | 34.38/33.68 | 35.22/34.15 |
| | Mean = 34.50/33.58 | Mean = 35.02/34.69 |
| VR 24193 10⁻⁵, | 33.77/34.06 | 33.66/34.26 |
| 35 μl beads | 33.76/33.80 | 34.12/34.76 |
| | Mean = 33.76/33.93 | Mean = 33.89/34.51 |

TABLE 24
crossing threshold for samples spiked into DB#5 or lysed
in easyMag lysis buffer, and then extracted with easyMag reagents.

| Well | Fluor | Target | Content | Sample | Cq |
|---|---|---|---|---|---|
| D01 | Cal Orange | Envelope | Unkn-4 | DB #5/MMM | 22.94 |
| D01 | FAM | UTR | Unkn-4 | DB #5/MMM | 23.57 |
| E01 | Cal Orange | Envelope | Unkn-4 | DB #5/MMM | 22.85 |
| E01 | FAM | UTR | Unkn-4 | DB #5/MMM | 23.65 |
| D02 | Cal Orange | Envelope | Unkn-5 | DB #5/MMM | 22.42 |
| D02 | FAM | UTR | Unkn-5 | DB #5/MMM | 23.25 |
| E02 | Cal Orange | Envelope | Unkn-5 | DB #5/MMM | 22.49 |
| E02 | FAM | UTR | Unkn-5 | DB #5/MMM | 23.21 |
| D03 | Cal Orange | Envelope | Unkn-6 | DB #5/MMM | 22.93 |
| D03 | FAM | UTR | Unkn-6 | DB #5/MMM | 23.44 |
| E03 | Cal Orange | Envelope | Unkn-6 | DB #5/MMM | 22.82 |
| E03 | FAM | UTR | Unkn-6 | DB #5/MMM | 23.40 |
| G01 | Cal Orange | Envelope | Unkn-7 | EasyMAG Lysis Buffer | 23.88 |
| G01 | FAM | UTR | Unkn-7 | EasyMAG Lysis Buffer | 24.81 |
| H01 | Cal Orange | Envelope | Unkn-7 | EasyMAG Lysis Buffer | 23.90 |
| H01 | FAM | UTR | Unkn-7 | EasyMAG Lysis Buffer | 24.81 |
| G02 | Cal Orange | Envelope | Unkn-8 | EasyMAG Lysis Buffer | 23.60 |
| G02 | FAM | UTR | Unkn-8 | EasyMAG Lysis Buffer | 24.63 |
| H02 | Cal Orange | Envelope | Unkn-8 | EasyMAG Lysis Buffer | 23.72 |
| H02 | FAM | UTR | Unkn-8 | EasyMAG Lysis Buffer | 24.68 |
| G03 | Cal Orange | Envelope | Unkn-9 | EasyMAG Lysis Buffer | 23.65 |
| G03 | FAM | UTR | Unkn-9 | EasyMAG Lysis Buffer | 24.57 |
| H03 | Cal Orange | Envelope | Unkn-9 | EasyMAG Lysis Buffer | 23.68 |
| H03 | FAM | UTR | Unkn-9 | EasyMAG Lysis Buffer | 24.65 |

TABLE 25
crossing thresholds for samples spiked into DB#5 variants stored
at 4 C., room temperature, or 37 C. for 0, 1, 3, 5, or 7 days.

| | 4 C. Storage | | | | | | | | | |
|---|---|---|---|---|---|---|---|---|---|---|
| | Day 0 | | Day 1 | | Day 3 | | Day 5 | | Day 7 | |
| | E gene | UTR | E gene | UTR | E gene | UTR | E gene | UTR | E gene | UTR |
| MMM #1 | 26.96 | 28.54 | 26.89 | 28.64 | 27.24 | 28.76 | 27.09 | 28.81 | 27.43 | 29.12 |
| MMM #1 | 26.89 | 28.50 | 26.63 | 28.39 | 26.86 | 28.28 | 26.79 | 28.45 | 27.20 | 28.64 |
| MMM #1 | 26.61 | 28.24 | 26.46 | 28.18 | 26.81 | 28.27 | 26.68 | 28.25 | 27.07 | 28.50 |
| MMM #1 | 26.73 | 28.36 | 27.26 | 29.17 | 27.21 | 28.72 | 27.15 | 28.96 | 26.84 | 28.35 |
| MEAN | 26.79 | 28.41 | 26.81 | 28.60 | 27.03 | 28.51 | 26.93 | 28.62 | 27.13 | 28.65 |
| MMM #2 | 27.08 | 28.69 | 26.67 | 28.28 | 26.91 | 28.34 | 26.90 | 28.50 | 27.36 | 28.77 |
| MMM #2 | 26.88 | 28.45 | 26.67 | 28.31 | 26.84 | 28.27 | 25.72 | 24.59 | 26.92 | 28.25 |
| MMM #2 | 26.92 | 28.49 | 26.71 | 28.35 | 26.95 | 28.36 | 26.86 | 28.40 | 26.82 | 28.31 |
| MMM #2 | 26.90 | 28.49 | 26.69 | 28.35 | 26.78 | 28.33 | 26.86 | 28.53 | 26.96 | 28.41 |
| MEAN | 26.94 | 28.53 | 26.69 | 28.32 | 26.87 | 28.32 | 26.58 | 27.50 | 27.01 | 28.43 |
| MMM #3 | 26.83 | 28.39 | 26.78 | 28.45 | 27.06 | 28.40 | 26.99 | 28.57 | 27.17 | 28.58 |
| MMM #3 | 26.86 | 28.39 | 26.69 | 28.39 | 27.07 | 28.43 | 26.78 | 28.40 | 27.02 | 28.47 |
| MMM #3 | 26.63 | 28.27 | 26.89 | 28.39 | 27.05 | 28.39 | 27.06 | 28.52 | 26.99 | 28.35 |
| MMM #3 | 26.62 | 28.24 | 26.63 | 28.24 | 26.75 | 28.26 | 26.71 | 28.38 | 26.76 | 28.16 |
| MEAN | 26.74 | 28.32 | 26.75 | 28.37 | 26.98 | 28.37 | 26.89 | 28.47 | 26.99 | 28.39 |
| MMM #4 | 26.67 | 28.28 | 26.84 | 28.40 | 27.14 | 28.58 | 27.01 | 28.51 | 26.85 | 28.31 |
| MMM #4 | 26.91 | 28.49 | 26.79 | 28.37 | 26.74 | 28.24 | 26.79 | 28.46 | 27.03 | 28.37 |
| MMM #4 | 26.89 | 28.45 | 26.78 | 28.39 | 27.00 | 28.42 | 26.73 | 28.28 | 27.15 | 28.53 |
| MMM #4 | 26.83 | 28.34 | 26.78 | 28.37 | 27.04 | 28.46 | 26.98 | 28.45 | 27.29 | 28.72 |
| MEAN | 26.82 | 28.39 | 26.80 | 28.38 | 26.98 | 28.43 | 26.88 | 28.43 | 27.08 | 28.48 |
| | Room Temperature Storage | | | | | | | | | |
| | Day 0 | | Day 1 | | Day 3 | | Day 5 | | Day 7 | |
| | E gene | UTR | E gene | UTR | E gene | UTR | E gene | UTR | E gene | UTR |
| MMM #1 | 26.96 | 28.54 | 26.65 | 28.37 | 26.74 | 28.57 | 26.58 | 28.36 | 27.23 | 28.87 |
| MMM #1 | 26.89 | 28.50 | 26.49 | 28.11 | 26.37 | 28.11 | 26.48 | 28.25 | 26.64 | 28.26 |
| MMM #1 | 26.61 | 28.24 | 26.46 | 28.07 | 26.52 | 28.20 | 26.43 | 28.20 | 26.65 | 28.14 |
| MMM #1 | 26.73 | 28.36 | 26.36 | 27.98 | 26.49 | 28.31 | 26.29 | 28.03 | 26.55 | 28.19 |
| MEAN | 26.79 | 28.41 | 26.49 | 28.13 | 26.53 | 28.30 | 26.44 | 28.21 | 26.76 | 28.37 |
| MMM #2 | 27.08 | 28.69 | 26.67 | 28.19 | 26.99 | 28.60 | 27.06 | 28.67 | 26.80 | 28.37 |
| MMM #2 | 26.88 | 28.45 | 26.60 | 28.18 | 26.66 | 28.30 | 26.57 | 28.23 | 26.88 | 28.34 |
| MMM #2 | 26.92 | 28.49 | 26.63 | 28.23 | 26.66 | 28.34 | 26.58 | 28.21 | 26.87 | 28.34 |
| MMM #2 | 26.90 | 28.49 | 26.73 | 28.25 | 26.77 | 28.40 | 26.68 | 28.38 | 27.01 | 28.50 |
| MEAN | 26.94 | 28.53 | 26.66 | 28.22 | 26.77 | 28.41 | 26.72 | 28.37 | 26.89 | 28.39 |
| MMM #3 | 26.83 | 28.39 | 26.78 | 28.39 | 27.06 | 28.53 | 27.14 | 28.84 | 27.11 | 28.61 |
| MMM #3 | 26.86 | 28.39 | 26.61 | 28.23 | 26.57 | 28.20 | 27.08 | 28.78 | 27.40 | 29.02 |
| MMM #3 | 26.63 | 28.27 | 26.63 | 28.15 | 26.88 | 28.38 | 26.88 | 28.47 | 26.95 | 28.35 |
| MMM #3 | 26.62 | 28.24 | 26.52 | 28.16 | 26.62 | 28.21 | 26.60 | 28.18 | 26.99 | 28.46 |
| MEAN | 26.74 | 28.32 | 26.64 | 28.23 | 26.78 | 28.33 | 26.93 | 28.57 | 27.11 | 28.61 |
| MMM #4 | 26.67 | 28.28 | 26.52 | 28.19 | 26.64 | 28.38 | 26.64 | 28.36 | 26.83 | 28.51 |
| MMM #4 | 26.91 | 28.49 | 26.71 | 28.20 | 26.64 | 28.36 | 26.69 | 28.41 | 26.84 | 28.42 |
| MMM #4 | 26.89 | 28.45 | 26.57 | 28.18 | 26.86 | 28.61 | 26.61 | 28.38 | 26.90 | 28.58 |
| MMM #4 | 26.83 | 28.34 | 26.55 | 28.16 | 26.69 | 28.50 | 25.98 | 27.54 | 26.87 | 28.45 |
| MEAN | 26.82 | 28.39 | 26.59 | 28.18 | 26.71 | 28.46 | 26.48 | 28.17 | 26.86 | 28.49 |
| | 37 C. Storage | | | | | | | | | |
| | Day 0 | | Day 1 | | Day 3 | | Day 5 | | Day 7 | |
| | E gene | UTR | E gene | UTR | E gene | UTR | E gene | UTR | E gene | UTR |
| MMM #1 | 26.96 | 28.54 | 26.37 | 28.14 | 26.53 | 28.30 | 27.25 | 26.05 | 27.25 | 29.03 |
| MMM #1 | 26.89 | 28.50 | 26.54 | 28.23 | 26.48 | 28.25 | 26.81 | 28.63 | 27.13 | 28.93 |
| MMM #1 | 26.61 | 28.24 | 26.38 | 28.06 | 26.53 | 28.17 | 26.77 | 28.68 | 27.19 | 28.87 |
| MMM #1 | 26.73 | 28.36 | 26.18 | 27.96 | 26.27 | 28.01 | 26.43 | 28.31 | 27.08 | 28.81 |
| MEAN | 26.79 | 28.41 | 26.37 | 28.10 | 26.45 | 28.18 | 26.81 | 27.92 | 27.16 | 28.91 |
| MMM #2 | 27.08 | 28.69 | 27.04 | 28.35 | 27.47 | 28.53 | 27.73 | 28.90 | 28.22 | 29.07 |
| MMM #2 | 26.88 | 28.45 | 26.93 | 28.18 | 27.37 | 28.23 | 27.40 | 28.55 | 28.18 | 29.12 |
| MMM #2 | 26.92 | 28.49 | 27.01 | 28.34 | 27.29 | 28.49 | 27.67 | 28.86 | 28.33 | 29.15 |
| MMM #2 | 26.90 | 28.49 | 27.06 | 28.48 | 27.32 | 28.46 | 27.62 | 28.74 | 28.23 | 29.17 |
| MEAN | 26.94 | 28.53 | 27.01 | 28.34 | 27.36 | 28.43 | 27.61 | 28.77 | 28.24 | 29.13 |
| MMM #3 | 26.83 | 28.39 | 27.28 | 28.87 | 27.76 | 28.66 | 28.44 | 29.31 | 29.25 | 29.79 |
| MMM #3 | 26.86 | 28.39 | 26.95 | 28.19 | 27.62 | 28.46 | 28.12 | 28.93 | 29.03 | 29.34 |
| MMM #3 | 26.63 | 28.27 | 26.88 | 28.20 | 27.47 | 28.35 | 28.25 | 29.04 | 28.99 | 29.19 |
| MMM #3 | 26.62 | 28.24 | 26.82 | 28.18 | 27.62 | 28.49 | 28.22 | 28.94 | 28.99 | 29.36 |
| MEAN | 26.74 | 28.32 | 26.98 | 28.36 | 27.62 | 28.49 | 28.26 | 29.06 | 29.07 | 29.42 |
| MMM #4 | 26.67 | 28.28 | 26.47 | 28.37 | 27.61 | 29.46 | 29.08 | 30.81 | 30.10 | 31.76 |
| MMM #4 | 26.91 | 28.49 | 26.46 | 28.17 | 27.65 | 29.66 | 29.03 | 31.01 | 29.99 | 31.58 |
| MMM #4 | 26.89 | 28.45 | 26.62 | 28.48 | 28.08 | 30.19 | 29.01 | 30.66 | 30.12 | 31.69 |
| MMM #4 | 26.83 | 28.34 | 26.60 | 28.36 | 27.80 | 29.78 | 28.76 | 30.63 | 30.22 | 31.78 |
| MEAN | 26.82 | 28.39 | 26.54 | 28.35 | 27.79 | 29.77 | 28.97 | 30.78 | 30.11 | 31.70 |

TABLE 26
crossing thresholds for Influenza A and SARS-CoV2 samples spiked into DB#5
(MMM) or UTM, stored at 4 C., room temperature, or 37 C. for 0, 1, 3, 5, or 7 days.

| | 4 C. Storage | | | | | | | | | | | | | | |
|---|---|---|---|---|---|---|---|---|---|---|---|---|---|---|---|
| | Day 0 | | | Day 1 | | | Day 3 | | | Day 5 | | | Day 7 | | |
| | | | RNase | | | RNase | | | RNase | | | RNase | | | RNase |
| | Flu A | COVID | P | Flu A | COVID | P | Flu A | COVID | P | Flu A | COVID | P | Flu A | COVID | P |
| MMM | 24.01 | 22.27 | 25.08 | 24.02 | 22.24 | 25.17 | 24.03 | 22.19 | 25.06 | 23.77 | 22.21 | 25.00 | 24.18 | 22.11 | 29.44 |
| MMM | 23.72 | 22.07 | 24.76 | 24.10 | 22.40 | 25.15 | 23.58 | 21.95 | 24.63 | 23.49 | 21.93 | 24.66 | 24.76 | 22.56 | 30.26 |
| MMM | 23.94 | 22.26 | 25.01 | 23.66 | 22.03 | 24.77 | 23.51 | 21.80 | 24.63 | 23.51 | 22.02 | 24.74 | 24.43 | 22.37 | 29.93 |
| MMM | 23.86 | 22.18 | 25.02 | 23.73 | 22.09 | 24.89 | 23.49 | 21.88 | 24.49 | 23.52 | 21.92 | 24.83 | 24.18 | 22.10 | 29.12 |
| MEAN | 23.88 | 22.19 | 24.97 | 23.88 | 22.19 | 25.00 | 23.65 | 21.96 | 24.70 | 23.57 | 22.02 | 24.81 | 24.39 | 22.29 | 29.69 |
| UTM | 23.89 | 22.53 | 25.82 | 23.96 | 22.49 | 25.84 | 23.87 | 22.17 | 25.72 | 23.92 | 22.62 | 25.59 | 25.06 | 23.86 | 30.66 |
| UTM | 24.03 | 22.27 | 25.42 | 23.88 | 22.51 | 25.86 | 24.05 | 22.34 | 25.40 | 23.70 | 22.54 | 26.07 | 25.57 | 24.14 | 31.02 |
| UTM | 24.32 | 22.58 | 25.87 | 23.90 | 22.34 | 25.15 | 24.24 | 23.05 | 25.70 | 23.74 | 22.73 | 25.85 | 24.60 | 23.19 | 31.48 |
| UTM | 24.09 | 22.66 | 25.47 | 24.22 | 22.98 | 25.81 | 24.04 | 22.50 | 25.57 | 23.86 | 22.50 | 25.97 | 24.46 | 23.20 | 30.80 |
| MEAN | 24.08 | 22.51 | 25.65 | 23.99 | 22.58 | 25.67 | 24.05 | 22.52 | 25.60 | 23.81 | 22.60 | 25.87 | 24.92 | 23.60 | 30.99 |
| | Room Temperature Storage | | | | | | | | | | | | | | |
| | Day 0 | | | Day 1 | | | Day 3 | | | Day 5 | | | Day 7 | | |
| | | | RNase | | | RNase | | | RNase | | | RNase | | | RNase |
| | Flu A | COVID | P | Flu A | COVID | P | Flu A | COVID | P | Flu A | COVID | P | Flu A | COVID | P |
| MMM | 24.01 | 22.27 | 25.08 | 23.65 | 22.02 | 24.80 | 24.28 | 22.24 | 26.26 | 23.71 | 22.06 | 24.77 | 24.63 | 22.34 | 29.19 |
| MMM | 23.72 | 22.07 | 24.76 | 23.59 | 22.00 | 24.89 | 24.28 | 22.28 | 26.27 | 23.72 | 22.06 | 24.91 | 24.37 | 22.13 | 28.85 |
| MMM | 23.94 | 22.26 | 25.01 | 23.49 | 22.00 | 24.61 | 24.07 | 22.19 | 25.95 | 23.69 | 22.08 | 24.81 | 24.43 | 22.24 | 28.88 |
| MMM | 23.86 | 22.18 | 25.02 | 23.66 | 22.03 | 24.61 | 24.29 | 22.33 | 26.29 | 23.48 | 21.92 | 24.70 | 24.75 | 22.36 | 28.84 |
| MEAN | 23.88 | 22.19 | 24.97 | 23.60 | 22.01 | 24.73 | 24.23 | 22.26 | 26.19 | 23.65 | 22.03 | 24.80 | 24.55 | 22.27 | 28.94 |
| UTM | 23.89 | 22.53 | 25.82 | 24.16 | 22.50 | 26.18 | 24.19 | 22.86 | 27.77 | 24.22 | 22.95 | 27.69 | 24.11 | 22.23 | 31.57 |
| UTM | 24.03 | 22.27 | 25.42 | 24.04 | 22.50 | 25.68 | 24.30 | 23.04 | 28.12 | 23.68 | 22.73 | 27.09 | 24.53 | 23.09 | 31.76 |
| UTM | 24.32 | 22.58 | 25.87 | 24.45 | 23.16 | 26.18 | 24.15 | 22.71 | 27.72 | 23.92 | 22.86 | 27.60 | 25.16 | 23.35 | 32.71 |
| UTM | 24.09 | 22.66 | 25.47 | 24.46 | 23.20 | 26.20 | 24.17 | 22.82 | 28.20 | 24.00 | 23.05 | 27.54 | 24.76 | 23.18 | 32.93 |
| MEAN | 24.08 | 22.51 | 25.65 | 24.28 | 22.84 | 26.06 | 24.20 | 22.86 | 27.95 | 23.96 | 22.90 | 27.48 | 24.64 | 22.96 | 32.24 |
| | 37 C. Storage | | | | | | | | | | | | | | |
| | Day 0 | | | Day 1 | | | Day 3 | | | Day 5 | | | Day 7 | | |
| | | | RNase | | | RNase | | | RNase | | | RNase | | | RNase |
| | Flu A | COVID | P | Flu A | COVID | P | Flu A | COVID | P | Flu A | COVID | P | Flu A | COVID | P |
| MMM | 24.01 | 22.27 | 25.08 | 23.91 | 22.08 | 24.91 | 24.65 | 22.61 | 24.95 | 24.74 | 22.80 | 25.07 | 26.15 | 23.32 | 28.95 |
| MMM | 23.72 | 22.07 | 24.76 | 23.73 | 21.90 | 24.71 | 24.38 | 22.34 | 24.71 | 24.50 | 22.60 | 24.81 | 25.72 | 23.28 | 28.60 |
| MMM | 23.94 | 22.26 | 25.01 | 23.76 | 22.03 | 25.16 | 24.34 | 22.36 | 24.65 | 24.41 | 22.58 | 24.66 | 25.69 | 23.21 | 28.33 |
| MMM | 23.86 | 22.18 | 25.02 | 23.73 | 22.00 | 24.79 | 24.26 | 22.28 | 24.56 | 24.49 | 22.50 | 24.68 | 25.98 | 23.25 | 28.37 |
| MEAN | 23.88 | 22.19 | 24.97 | 23.78 | 22.00 | 24.89 | 24.41 | 22.40 | 24.72 | 24.53 | 22.62 | 24.81 | 25.89 | 23.27 | 28.56 |
| UTM | 23.89 | 22.53 | 25.82 | 24.08 | 22.49 | 26.57 | 24.75 | 23.19 | 29.56 | 24.74 | 23.01 | 30.19 | 25.06 | 22.81 | 36.36 |
| UTM | 24.03 | 22.27 | 25.42 | 24.33 | 22.85 | 26.63 | 24.63 | 23.00 | 29.28 | 24.36 | 22.53 | 30.34 | 24.69 | 22.52 | 35.12 |
| UTM | 24.32 | 22.58 | 25.87 | 24.51 | 23.26 | 26.37 | 24.72 | 23.23 | 28.72 | 24.31 | 22.29 | 28.34 | 24.82 | 22.86 | 35.21 |
| UTM | 24.09 | 22.66 | 25.47 | 24.21 | 22.55 | 25.87 | 24.80 | 23.24 | 28.06 | 24.42 | 22.77 | 30.79 | 25.09 | 22.72 | 36.21 |
| MEAN | 24.08 | 22.51 | 25.65 | 24.28 | 22.79 | 26.36 | 24.73 | 23.16 | 28.91 | 24.46 | 22.65 | 29.92 | 24.92 | 22.73 | 35.73 |

TABLE 27
crossing thresholds for SARS-CoV2 samples spiked into DB#5 (MMM) or
UTM, stored at 4 C., room temperature, or 37 C. for 0, 1, 3, 5, or 7 days.

| | 4 C. Storage | | | | | | | | | | | | | | |
|---|---|---|---|---|---|---|---|---|---|---|---|---|---|---|---|
| | Day 0 | | | Day 1 | | | Day 3 | | | Day 5 | | | Day 7 | | |
| | | | RNase | | | RNase | | | RNase | | | RNase | | | RNase |
| | E gene | UTR | P | E gene | UTR | P | E gene | UTR | P | E gene | UTR | P | E gene | UTR | P |
| MMM | 22.09 | 23.91 | 26.50 | 22.04 | 23.67 | 24.77 | 21.75 | 23.51 | 24.60 | 22.05 | 23.74 | 26.67 | 22.11 | 23.61 | 25.31 |
| MMM | 22.04 | 23.66 | 26.41 | 22.20 | 23.98 | 26.94 | 21.78 | 23.47 | 26.24 | 22.00 | 23.76 | 26.45 | 22.13 | 23.59 | 25.42 |
| MMM | 22.04 | 23.52 | 26.64 | 22.00 | 23.59 | 26.89 | 21.76 | 23.47 | 26.33 | 21.88 | 23.59 | 26.53 | 22.20 | 23.68 | 25.65 |
| MMM | 21.98 | 23.57 | 26.40 | 21.97 | 23.55 | 26.88 | 21.68 | 23.34 | 26.16 | 21.88 | 23.63 | 26.54 | 22.12 | 23.68 | 25.17 |
| MEAN | 22.04 | 23.67 | 26.49 | 22.05 | 23.70 | 26.37 | 21.74 | 23.44 | 25.84 | 21.96 | 23.68 | 26.55 | 22.14 | 23.64 | 25.39 |
| UTM | 22.41 | 24.14 | 27.61 | 22.31 | 24.04 | 27.92 | 22.07 | 23.80 | 27.41 | 22.51 | 24.28 | 27.13 | 23.76 | 25.22 | 26.34 |
| UTM | 22.07 | 23.77 | 26.91 | 22.36 | 24.05 | 27.74 | 22.20 | 24.06 | 27.08 | 22.38 | 24.28 | 27.78 | 24.00 | 25.51 | 27.02 |
| UTM | 22.14 | 28.85 | 27.13 | 22.29 | 23.86 | 27.12 | 22.80 | 24.61 | 27.16 | 22.51 | 24.38 | 27.53 | 22.72 | 24.31 | 26.19 |
| UTM | 22.16 | 24.02 | 27.08 | 23.08 | 25.15 | 27.90 | 22.30 | 24.14 | 25.15 | 22.32 | 24.20 | 27.65 | 23.22 | 24.85 | 26.44 |
| MEAN | 22.20 | 23.95 | 27.18 | 22.51 | 24.28 | 27.67 | 22.34 | 24.15 | 26.70 | 22.43 | 24.29 | 27.52 | 23.43 | 24.97 | 26.50 |
| | Room Temperature Storage | | | | | | | | | | | | | | |
| | Day 0 | | | Day 1 | | | Day 3 | | | Day 5 | | | Day 7 | | |
| | | | RNase | | | RNase | | | RNase | | | RNase | | | RNase |
| | E gene | UTR | P | E gene | UTR | P | E gene | UTR | P | E gene | UTR | P | E gene | UTR | P |
| MMM | 22.09 | 23.91 | 26.50 | 21.87 | 23.53 | 26.65 | 22.02 | 23.71 | 26.32 | 21.94 | 23.76 | 26.58 | 22.18 | 23.81 | 25.15 |
| MMM | 22.04 | 23.66 | 26.41 | 21.88 | 23.65 | 26.85 | 21.99 | 23.59 | 26.35 | 21.95 | 23.81 | 26.75 | 22.07 | 23.66 | 25.20 |
| MMM | 22.04 | 23.52 | 26.64 | 21.91 | 23.53 | 26.66 | 21.94 | 23.50 | 26.23 | 21.79 | 23.57 | 26.34 | 22.07 | 23.74 | 25.04 |
| MMM | 21.98 | 23.57 | 26.40 | 21.87 | 23.41 | 26.56 | 22.22 | 23.95 | 26.41 | 21.77 | 23.50 | 26.29 | 22.12 | 23.63 | 25.16 |
| MEAN | 22.04 | 23.67 | 26.49 | 21.88 | 23.53 | 26.68 | 22.04 | 23.69 | 26.33 | 21.86 | 23.66 | 26.49 | 22.11 | 23.71 | 25.14 |
| UTM | 22.41 | 24.14 | 27.61 | 22.35 | 24.06 | 28.39 | 22.83 | 24.59 | 28.63 | 22.87 | 24.56 | 30.24 | 22.25 | 23.99 | 27.15 |
| UTM | 22.07 | 23.77 | 26.91 | 22.32 | 24.11 | 27.30 | 23.05 | 24.77 | 28.91 | 22.69 | 24.51 | 29.24 | 23.01 | 24.74 | 27.51 |
| UTM | 22.14 | 28.85 | 27.13 | 22.90 | 24.63 | 27.76 | 22.70 | 24.50 | 28.86 | 22.65 | 24.45 | 29.87 | 23.38 | 25.06 | 28.04 |
| UTM | 22.16 | 24.02 | 27.08 | 23.04 | 24.40 | 27.74 | 22.82 | 24.69 | 29.26 | 22.73 | 24.54 | 29.76 | 23.09 | 24.61 | 27.56 |
| MEAN | 22.20 | 23.95 | 27.18 | 22.65 | 24.30 | 27.80 | 22.85 | 24.64 | 28.92 | 22.73 | 24.52 | 29.78 | 22.93 | 24.60 | 27.56 |
| | 37 C. Storage | | | | | | | | | | | | | | |
| | Day 0 | | | Day 1 | | | Day 3 | | | Day 5 | | | Day 7 | | |
| | | | RNase | | | RNase | | | RNase | | | RNase | | | RNase |
| | E gene | UTR | P | E gene | UTR | P | E gene | UTR | P | E gene | UTR | P | E gene | UTR | P |
| MMM | 22.09 | 23.91 | 26.50 | 21.98 | 23.69 | 26.74 | 22.39 | 24.10 | 26.11 | 22.64 | 24.30 | 26.39 | 23.47 | 24.63 | 25.31 |
| MMM | 22.04 | 23.66 | 26.41 | 21.83 | 23.52 | 26.47 | 22.15 | 23.77 | 26.00 | 22.67 | 24.17 | 26.48 | 23.33 | 24.37 | 25.24 |
| MMM | 22.04 | 23.52 | 26.64 | 22.04 | 23.79 | 26.41 | 22.16 | 23.85 | 25.99 | 22.52 | 24.06 | 26.22 | 23.29 | 24.49 | 25.18 |
| MMM | 21.98 | 23.57 | 26.40 | 21.81 | 23.45 | 26.46 | 22.11 | 23.71 | 25.84 | 22.49 | 24.05 | 26.14 | 23.29 | 24.51 | 25.21 |
| MEAN | 22.04 | 23.67 | 26.49 | 21.92 | 23.61 | 26.52 | 22.20 | 23.86 | 25.98 | 22.58 | 24.14 | 26.31 | 23.35 | 24.50 | 25.23 |
| UTM | 22.41 | 24.14 | 27.61 | 22.40 | 24.34 | 28.77 | 23.06 | 25.16 | 32.40 | 22.86 | 25.06 | 36.84 | 22.69 | 24.77 | 31.17 |
| UTM | 22.07 | 23.77 | 26.91 | 22.62 | 24.50 | 28.64 | 22.84 | 25.01 | 33.51 | 22.53 | 24.76 | 37.12 | 22.50 | 24.50 | 30.05 |
| UTM | 22.14 | 28.85 | 27.13 | 23.27 | 25.57 | 28.53 | 23.11 | 25.23 | 31.06 | 22.28 | 24.59 | 32.66 | 22.97 | 25.12 | 30.17 |
| UTM | 22.16 | 24.02 | 27.08 | 22.28 | 24.24 | 28.77 | 23.09 | 26.06 | 30.47 | 22.68 | 25.06 | 38.68 | 22.64 | 24.71 | 30.73 |
| MEAN | 22.20 | 23.95 | 27.18 | 22.64 | 24.66 | 28.68 | 23.03 | 25.37 | 31.86 | 22.58 | 24.87 | 36.33 | 22.70 | 24.78 | 30.53 |

TABLE 28
crossing thresholds for Group A Streptococcus samples spiked into DB#5
(MMM) or UTM, stored at 4 C., room temperature, or 37 C. for 0, 1, 3, 5, or 7 days.

| | | Day 1 | | | Day 3 | | | Day 5 | | | Day 7 | | |
|---|---|---|---|---|---|---|---|---|---|---|---|---|---|
| | | | Room | | | Room | | | Room | | | Room | |
| | Day 0 | 4 C. | Temp | 37 C. | 4 C. | Temp | 37 C. | 4 C. | Temp | 37 C. | 4 C. | Temp | 37 C. |
| MMM | 26.50 | 25.32 | 25.07 | 25.71 | 24.33 | 25.19 | 26.21 | 25.46 | 25.62 | 25.46 | 26.00 | 25.15 | 25.96 |
| MMM | 26.41 | 25.86 | 26.14 | 26.01 | 25.09 | 24.99 | 24.71 | 25.40 | 26.12 | 25.68 | 26.25 | 25.24 | 25.89 |
| MMM | 26.64 | 26.13 | 25.63 | 25.17 | 25.22 | 24.31 | 24.94 | 25.54 | 25.54 | 25.18 | 28.32 | 25.67 | 26.36 |
| MMM | 26.40 | 27.51 | 25.61 | 27.22 | 25.08 | 25.11 | 25.31 | 25.78 | 25.62 | 25.17 | 26.78 | 25.89 | 27.12 |
| MEAN | 26.49 | 26.21 | 25.61 | 26.03 | 24.93 | 24.90 | 25.29 | 25.54 | 25.72 | 25.37 | 26.84 | 25.49 | 26.33 |
| UTM | 28.18 | 28.08 | 29.85 | 30.85 | 28.14 | 28.40 | 31.41 | 28.23 | 29.85 | 30.60 | 30.66 | 29.03 | 32.50 |
| UTM | 28.26 | 28.05 | 29.13 | 31.31 | 28.19 | 28.48 | 30.94 | 29.13 | 29.54 | 30.45 | 32.08 | 29.89 | 28.52 |
| UTM | 28 | 28.34 | 30.26 | 31.12 | 28.27 | 27.97 | 30.87 | 29.62 | 29.76 | 30.31 | 28.59 | 29.14 | 30.31 |
| UTM | 28.05 | 29.34 | 30.30 | 30.87 | 28.78 | 28.18 | 30.54 | 29.27 | 30.22 | 30.70 | 28.68 | 29.36 | 31.48 |
| MEAN | 28.12 | 28.45 | 29.89 | 31.04 | 28.35 | 28.26 | 30.94 | 29.06 | 29.84 | 30.51 | 30.00 | 29.36 | 30.70 |

1. An aqueous composition with a pH between about 6 and about 7 at about 20° C., and comprising:
a) about 2-6M of a chaotropic agent,
b) about 0.01-0.1M of a chelator,
c) about 0.5%-3.0% of a detergent,
d) about 0.02-0.1M of a buffer, and
e) about 5%-40% of an alcohol,
wherein the composition does not comprise a reducing agent and wherein the composition does not comprise a mucolytic agent, and wherein the composition inactivates pathogens and stabilizes nucleic acids thereof contained within a sample suspected of containing a pathogen.

2. (canceled)

3. The composition of claim 1, wherein the chaotropic agent is selected from guanidine hydrochloride, guanidine thiocyanate, urea or a combination thereof; the chelator is selected from EDTA, EGTA, sodium citrate or a combination thereof; and/or the buffer is sodium citrate or Tris-HCl.

4. The composition of claim 1, wherein the chaotropic agent is guanidine thiocyanate; the chelator is EDTA; the detergent is Triton X-100; the buffer is Tris-HCl; the alcohol is isopropanol; and/or the pH of the composition is about 7.0 at about 20° C.

5.-19. (canceled)

20. The composition of claim 1, wherein the composition comprises about 4M guanidine thiocyanate, about 2.0% Triton X-100, about 0.04M EDTA, about 0.05M Tris-HCl, and about 20% isopropanol, and has a pH of about 7.0 at 20° C.

21. The composition of claim 1, wherein the composition comprises about 3M guanidine thiocyanate, about 2.0% Triton X-100, about 0.04M EDTA, about 0.05M Tris-HCl, and about 20% isopropanol, and has a pH of about 7.0 at 20° C.

22. The composition of claim 1, wherein the composition comprises about 2M guanidine thiocyanate, about 2.0% Triton X-100, about 0.04M EDTA, about 0.05M Tris-HCl, and about 20% isopropanol, and has a pH of about 7.0 at 20° C.

23. The composition of claim 1, wherein the composition comprises about 2M guanidine thiocyanate, about 1.0% Triton X-100, about 0.02M EDTA, about 0.025M Tris-HCl, and about 10% isopropanol, and has a pH of about 7.0 at 20° C.

24. The composition claim 1, further comprising a foam suppressor, optionally Antifoam A.

25. The composition of claim 1, wherein the pathogen is a bacterium, mycobacterium, fungus, or virus.

26.-31. (canceled)

32. A method of inactivating pathogens and stabilizing nucleic acids and/or proteins thereof contained within a sample suspected of containing a pathogen, the method comprising:
a) obtaining a sample suspected of containing a pathogen;
b) contacting the sample with the composition of claim 1; and
c) incubating the sample contacted with the composition under conditions to allow for inactivation of pathogens and stabilization of nucleic acids and/or proteins.

33. The method of claim 32, wherein the sample is a nasal swab, mid-turbinate swab, nasopharyngeal swab, nasal sponge, nasal wash, oral swab, buccal swab, throat swab, oral wash or gargle, oropharyngeal swab, combined oral and nasal swab, combined throat and nasal swab, skin swab, rectal swab, stool swab, skin scraping, nail clipping, urine sample, blood sample, saliva sample, vaginal swab, meatal swab, or urethral swab.

34. (canceled)

35. The method of claim 32, wherein the pathogen is selected from a bacteria, mycobacteria, fungi, and virus.

36. (canceled)

37. The method of claim 32, wherein the sample is incubated with the composition in step c) for at least about 10 minutes, at least about 15 minutes, or at least about 30 minutes, and/or at a temperature between about 4° C. and about 100° C., optionally about 4° C., about 10° C., about 15° C., about 20° C., about 25° C., about 30° C., about 37° C., about 42° C., about 65° C., about 72° C., about 95° C., or about 100° C.

38. (canceled)

39. A method of extracting nucleic acids contained within a sample suspected of containing a pathogen, the method comprising: inactivating pathogens and stabilizing nucleic acids contained within a sample suspected of containing a pathogen using the method of claim 32, and further comprising:
d) extracting the nucleic acids directly from the sample contacted with the composition.

40. The method of claim 39, wherein the nucleic acids are extracted using easyMag reagents, and/or the extracting comprises binding the nucleic acids to silica.

41. (canceled)

42. The method of claim 39, wherein the sample is stored for up to about 14 days, up to about 30 days, up to about 60 days, up to about 90 days, up to about 120 days, or up to about 180 days at a temperature of between about 4° C. to about 25° C. prior to extraction, or the sample is stored for up to about 14 days at a temperature of between about 4° C. to about 37° C. prior to extraction.

43. (canceled)

44. A method of detecting nucleic acids contained within a sample suspected of containing a pathogen, the method comprising: extracting nucleic acids contained within a sample suspected of containing a pathogen using the method of claim 39, and further comprising:
e) detecting the nucleic acids.

45. The method of claim 44, wherein the detection comprises a PCR-based method.

46. The method of claim 39, to wherein at least two samples are pooled prior to extraction.

47. A kit for collection, transport and/or storage of a sample suspected of containing a pathogen, the kit comprising: the composition of claim 1, and one or more components selected from a container, a collection device, and a wash solution.

48.-58. (canceled)
